(19) United States
(12) Patent Application Publication
(10) Pub. No.: US 2022/0402900 A1
(43) Pub. Date: Dec. 22, 2022

(54) 1,2,4-OXADIAZOL-5-ONE DERIVATIVES FOR THE TREATMENT OF CANCER

(71) Applicant: Basilea Pharmaceutica International AG, Basel (CH)

(72) Inventors: Florian RICHALET, Basel (CH); Sven WEILER, Basel (CH); Stefan REINELT, Basel (CH); Anna GRONER, Basel (CH); Heidi LANE, Basel (CH); Claude NUOFFER, Basel (CH)

(21) Appl. No.: 17/629,829

(22) PCT Filed: Jul. 28, 2020
(86) PCT No.: PCT/EP2020/071216

§ 371 Date: Jan. 25, 2022

(30) Foreign Application Priority Data
Jul. 29, 2019 (EP) 19188935.1
Feb. 6, 2020 (EP) 20155922.6
Mar. 10, 2020 (EP) 20162120.8

(51) Int. Cl. C07D 413/04; C07D 417/14; C07D 413/14; A61P 35/00

(52) U.S. Cl. CPC C07D 413/04; C07D 417/14; C07D 413/14; A61P 35/00

(57) ABSTRACT

The invention provides compounds of formula (I) and pharmaceutically acceptable salts thereof, wherein A1 is —N═ or —C(R3)=; A2 is —N═ or —CH═; L is —NH—; B1 and B2 are independently —N═ or —C(R2b)=; B3 and B4 are independently —C(R2b)=; no more than one R2b on B1, B2, B3 and B4 is other than hydrogen; R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl); R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂; R1a and R1b may together form a —CH═CH—CH═CH— moiety in which one or two non-adjacent CH are optionally replaced by N; R2a is halogen, C1-C6alkyl, C1-C6haloalkyl, cyclopropyl, cyclobutyl, —OR6, —NHC(═O)—C3-C6cycloalkyl, Cycle Q, —SF₅ or group Y (formula (Y)); wherein X is a 3- or 4-membered carbocyclic ring and R8 is halogen, cyano, C1-C6alkyl, C1-C6haloalkyl or —C1-C6alkyl-CN; and wherein R2b, R3, R4, R6 and R8 are as defined in the claims; as well as methods of using the compounds to treat neoplastic diseases, in particular cancer.

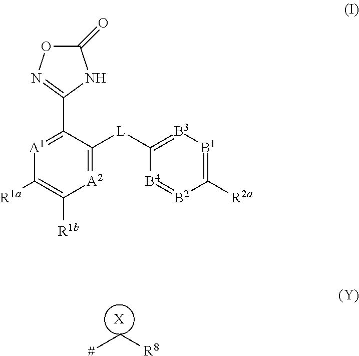

The present invention relates to compounds targeting the Hippo pathway, e.g. YAP/TAZ and/or the TEAD family, and their use in the treatment of neoplastic diseases such as cancer.

The Hippo pathway plays a conserved role in cell proliferation and organ size. YAP and TAZ are transcriptional co-activators, negatively regulated by the Hippo pathway. Thus, when the Hippo pathway is off, YAP and TAZ can translocate to the nucleus. To further function in transcriptional activation, YAP/TAZ work together with the transcriptional enhancer associated domain (TEAD) transcription factor family. Constitutive activity of YAP/TAZ and/or the TEAD family is present in different tumor types, consistent with these factors driving the expression of growth-promoting genes not only during development but also in cancer (See Holden and Cunningham “Targeting the Hippo Pathway and Cancer through the TEAD Family of Transcription Factors”, Cancers, 2018, 10, 81; Liu-Chittenden et al. “Genetic and pharmacological disruption of the TEAD-YAP complex suppresses the oncogenic activity of YAP”, Genes & Development, 2012, 26, 1300-1305; Santucci et al. “The Hippo Pathway and YAP/TAZ-TEAD Protein-Protein Interaction as Targets for Regenerative Medicine and Cancer Treatment”, J. Med. Chem., 2015, 58, 4857-4873; Pobbati et al. “Targeting the Central Pocket in Human Transcription Factor TEAD as a Potential Cancer Therapeutic Strategy”, Structure, 2015, 23(11), 2076-2086). Therefore, targeting of YAP/TAZ and/or the TEAD family harbors potential for anti-cancer therapy.

WO2013/188138, WO2015/022283, WO2015/063747, WO2017/064277, WO2018/204532, WO2018/185266, WO2019/040380 and WO2019/113236 describe inhibitors associated with one or more members of the Hippo pathway network, such as inhibitors of YAP/TAZ or inhibitors that modulate the interaction between YAP/TAZ and TEAD.

In a first aspect the present invention provides compounds of formula (I)

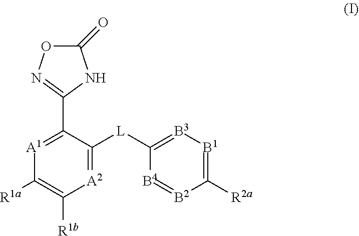

and pharmaceutically acceptable salts thereof, wherein

A1 is —N═ or —C(R3)=;

A2 is —N═ or —CH═;

L is —NH—;

B1 and B2 are independently —N═ or —C(R2b)=;

B3 and B4 are independently —C(R2b)=;

no more than one R2b on B1, B2, B3 and B4 is other than hydrogen;

R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);

R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;

R1a and R1b may together form a —CH═CH—CH═CH— moiety in which one or two non-adjacent CH are optionally replaced by N;

R2a is halogen, C1-C6alkyl, C1-C6haloalkyl, cyclopropyl, cyclobutyl, —OR6, —NHC(═O)—C3-C6cycloalkyl, Cycle Q, —SF₅ or group Y

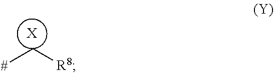

wherein X is a 3- or 4-membered carbocyclic ring and R8 is halogen, cyano, C1-C6alkyl, C1-C6haloalkyl or —C1-C6alkyl-CN;

R2b is hydrogen, halogen, methyl, —NH₂, halomethyl, —OCH₃ or —O-halomethyl;

R3 is hydrogen, halogen, C1-C6alkyl, —OC1-C6alkyl, —O—Cycle P, —O—Cycle Q1, —C1-C6alkyl-R9, —OC1-C6alkyl-R9, wherein in each alkyl group or moiety in the foregoing one non-terminal —CH₂— may be replaced by —NH— or —O— and wherein each alkyl group or moiety in the foregoing may be substituted by one or more halogen, wherein R9 is halogen, cyano, hydroxyl, —OC1-C2alkyl, —NH₂, —NH(C1-C2alkyl), —N(C1-C2alkyl)₂, —C(═O)NH₂, —C(═O)NH(C1-C2alkyl), —C(═O)N(C1-C2alkyl)₂, —C(═O)OH, —C(═O)O—C1-C2alkyl, —C(═O)—C1-C2alkyl, —NH(C═O)—C1-C2alkyl, —NH—S(O)₂—C1-C2alkyl, Cycle P or Cycle Q1;

R1a and R3 may together form a —CH₂—CH₂—CH₂— or a —CH₂—CH₂—CH₂—CH₂— moiety in which one of the —CH₂— units is optionally replaced by —NH—;

R4 is —OH or —NH₂;

R6 is C1-C6alkyl, C1-C6haloalkyl or —C1-C4alkylene-C3-C6cycloalkyl;

Cycle P is a 3- to 6-membered carbocyclic ring or a 3- to 6-membered heterocyclic ring, each optionally substituted by one to three R10;

Cycle Q is phenyl or 5-6 membered heteroaryl, each optionally substituted by one to three R7;

Cycle Q1 is phenyl or 5-6 membered heteroaryl, each optionally substituted by one to three R7a; and each R7, R7a and R10 is independently C1-C4alkyl.

In a further aspect, the invention provides compounds of formula (I) and pharmaceutically acceptable salts thereof for use in the treatment of neoplastic diseases in a subject selected from a mammal, in particular a human.

In a further aspect, the invention provides use of compounds of formula (I) and pharmaceutically acceptable salts thereof in the manufacture of a medicament for the treatment of neoplastic diseases in a subject selected from a mammal, in particular a human.

In a further aspect, the invention provides methods of treating neoplastic diseases in a subject selected from a mammal, in particular a human, comprising administering a compound of formula (I) or pharmaceutically acceptable salt thereof, e.g. in a therapeutically acceptable amount, to said subject.

In a further aspect, the invention provides pharmaceutical compositions comprising a compound of formula (I) or pharmaceutically acceptable salt thereof and one or more pharmaceutically acceptable excipients.

Each alkyl moiety either alone or as part of a larger group such as alkoxy is a straight or branched chain, unless otherwise stated. Examples include methyl, ethyl, n-propyl, prop-2-yl, n-butyl, but-2-yl, 2-methyl-prop-1-yl or 2-methyl-prop-2-yl. Alkyl groups stated as being “n-alkyl” are straight chain alkyl groups and not branched.

Each haloalkyl moiety either alone or as part of a larger group such as haloalkoxy is an alkyl group substituted by one or more of the same or different halogen atoms. Examples include difluoromethyl, trifluoromethyl, chlorodifluoromethyl and 2,2,2-trifluoro-ethyl. Haloalkyl moieties include for example 1 to 5 halo substituents, or 1 to 3 halo substituents.

The terms cycloalkyl and carbocyclic ring are synonymous and refer to saturated groups. Examples include cyclopropyl, cyclobutyl, cyclopentyl and cyclohexyl.

Halogen is fluorine, chlorine, bromine, or iodine.

Heteroaryl refers to an aromatic ring system containing at least one heteroatom, and preferably up to four, more preferably three, heteroatoms selected from nitrogen, oxygen and sulfur as ring members.

Heteroaryl rings do not contain adjacent oxygen atoms, adjacent sulfur atoms, or adjacent oxygen and sulfur atoms within the ring. Examples include pyridinyl, pyrimidinyl, pyrazinyl, pyridazinyl, pyrrolyl, pyrazolyl, imidazolyl, triazolyl, isoxazolyl, oxazolyl, oxadiazolyl, isothiazolyl, thiazolyl, thiadiazolyl, tetrazolyl, furanyl and thiophenyl.

Heterocyclic ring refers to a saturated or partially unsaturated carbocyclic ring containing one to four heteroatoms selected from nitrogen, oxygen and sulfur as ring members. Such rings do not contain adjacent oxygen atoms, adjacent sulfur atoms, or adjacent oxygen and sulfur atoms within the ring.

Examples include tetrahydrofuranyl, azetidinyl, pyrrolidinyl, piperidinyl, piperazinyl, tetrahydropyran, dioxanyl and morpholinyl.

Where a group is said to be optionally substituted, it may be substituted or unsubstituted, for example optionally with 1-5 substituents, for example optionally with 1-3 substituents.

The compounds of the invention also include all tautomeric forms of the compounds of formula (I). For example the 4H-1,2,4-oxadiazole-5-one moiety exhibits tautomeric forms as shown below. All forms are included within the scope of the compounds of formula (I).

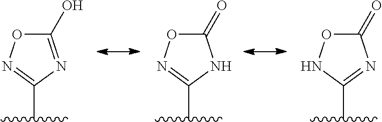 [SMILES: *c1noc(O)n1 ; *c1noc(=O)[nH]1 ; *c1nc(=O)o[nH]1]

The compounds of formula (I) may also be solvated, especially hydrated, which are also included in the compounds of formula (I). Solvation and hydration may take place during the preparation process.

Reference to compounds of the invention includes pharmaceutically acceptable salts of said compounds. Such salts may also exist as hydrates and solvates. Examples of pharmacologically acceptable salts of the compounds of formula (I) are salts of physiologically acceptable mineral acids, such as hydrochloric acid, sulfuric acid and phosphoric acid, or salts of organic acids, such as methane-sulfonic acid, p-toluenesulfonic acid, lactic acid, acetic acid, trifluoroacetic acid, citric acid, succinic acid, fumaric acid, maleic acid and salicylic acid. Further examples of pharmacologically acceptable salts of the compounds of formula (I) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts.

The following examples of substituent definitions and embodiments may be combined in any combination.

A1 is —N═ or —C(R3)=. Specific examples of A1 include —N═, —CH═, —C(CH₃)═, —C(F)═, —C(OCH₃)═, —C(—O(tetrahydropyran-4-yl))=, —C(O(CH₂)₂(pyridin-2-yl))=, —C(O(CH₂)₂(morpholin-4-yl))=, —C(O(CH₂)₂NH(CH₂)₂OH)═, —C(O-(piperid-4-yl))=, —C(O-(tert-butyl))=, —C(OCH(CH₃)₂)═, —C(OCH₂(1-methylcyclopropyl)=, —C(OCH₂CH₂C(CH₃)₂OCH₃)═, —C(OCH₂CH₂F)═, —C(O—CH₂CH₂NH₂)═, —C(O—CH₂CH₂NH—C(═O)CH₃)═, —C(O—CH₂CH₂NH—SO₂—CH₃)═, —C(OCH₂CH₂OCH₃)═, —C(OCH₂CH₂OH)═, —C(O—CH₂C(═O)NH₂)═ and —C(O—CH₂C(═O)OH)═. Moreover, A1 may be —C(R3)=, wherein R3 and R1a together form a —CH₂—CH₂—CH₂— or a —CH₂—CH₂—CH₂—CH₂— moiety in which one of the —CH₂— units is optionally replaced by —NH—.

A2 is —N═ or —CH═.

In some embodiments at least one of A1 and A2 is —N═.

In some embodiments A1 is —N═.

In some embodiments A1 is —C(R3)=.

In some embodiments A2 is —N═.

In some embodiments A2 is —CH═.

In some embodiments A1 is —N═ and A2 is —N═.

In some embodiments A1 is —N═ and A2 is —CH═.

In some embodiments A1 is —C(R3)= and A2 is —N═.

In some embodiments A1 is —C(R3)=, A2 is —N═ and R3 is other than hydrogen.

In some embodiments A1 is —C(R3)= and A2 is —CH═.

L is —NH—.

B1 and B2 are independently —N═ or —C(R2b)=, B3 and B4 are independently —C(R2b)=, wherein no more than one R2b on B1, B2, B3 and B4 is other than hydrogen. In particular, B1 is —N═ or —C(R2b)=, B2 is —N═ or —C(R2b)=, B3 is —C(R2b)= and B4 is —C(R2b)=.

Specific examples of B1 include —N═, —CH═, —C(F)═, —C(Cl)═, —C(OCH₃)═, —C(CF₃)═ and —C(NH₂)═, e.g. —N═, —CH═ or —C(OCH₃)═.

Specific examples of B2 include —N═, —CH═, —C(F)═, —C(Cl)═, —C(OCH₃)═, —C(CF₃)═ and —C(NH₂)═, e.g. —N═ or —CH═.

Specific examples of B3 include CH═, —C(Cl)═, —C(F)═, —C(OCH₃)═ and —C(NH₂)═, e.g. —CH═ or —C(OCH₃)═.

Specific examples of B4 include —CH═, —C(Cl)═, —C(F)═, —C(OCH₃)═ and —C(NH₂)═, e.g. —CH═

In some embodiments B1, B2, B3 and B4 are —CH═.

In some embodiments B1 and B2 are —N═ and B3 and B4 are —CH═.

In some embodiments B1 is —CH═, B2 is —N═ and B3 and B4 are —CH═.

In some embodiments B1, B2 and B4 are —CH═ and B3 is —C(R2b)=, wherein R2b is other than hydrogen.

In some embodiments B2, B3 and B4 are —CH═ and B1 is —C(R2b)=, wherein R2b is other than hydrogen.

R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl), preferably hydrogen, halogen (e.g. chloro or fluoro), C1-C3alkyl (n-alkyl), C1-C3alkyl(n-alkyl)-NH₂, C1-C3alkyl(n-alkyl)-OH, —NH₂, —NH(C1-C3alkyl (n-alkyl)), —NH(C(═O)—C1-C2alkyl), —OC1-C3alkyl (n-alkyl) or C1-C3haloalkyl (n-alkyl), in particular hydrogen, chloro, methyl or —NH₂. Specific examples of R1a include hydrogen, chloro, methyl, ethyl, trifluoromethyl, methoxy, —CH₂—OH, —NH—CH₃, —CH₂CH₂—NH₂, —NH₂ and —NH—C(═O)—CH₃.

In some embodiments R1a and R3 together form a —CH₂—CH₂—CH₂— or a —CH₂—CH₂—CH₂—CH₂— moiety in which one of the —CH₂— units (i.e. in the —CH₂—CH₂—CH₂— or —CH₂—CH₂—CH₂—CH₂— moiety) is optionally replaced by —NH—; in other embodiments R1a and R3 together form a —CH₂—CH₂—CH₂— moiety.

R1b is hydrogen, halogen (e.g. chloro or fluoro), C1-C3alkyl (n-alkyl), —OC1-C3alkyl (n-alkyl), —NH₂, —NH(C1-C3alkyl (n-alkyl)) or —N(C1-C3alkyl (n-alkyl))₂, preferably hydrogen, C1-C3alkyl (n-alkyl), —OC1-C3alkyl (n-alkyl) or —NH₂. Specific examples of R1b include hydrogen, methyl, methoxy and —NH₂.

In some embodiments at least one of R1a and R1b is hydrogen.

In some embodiments R1a and R1b together form a —CH═CH—CH═CH— moiety, in which one or two non-adjacent CH may be optionally replaced by N.

In some embodiments R1a and R1b together form a —CH═CH—CH═CH— moiety.

In some embodiments at least one of R1a and R1b is hydrogen or R1a and R1b together form a —CH═CH—CH═CH— moiety.

R2a is halogen, C1-C6alkyl, C1-C6haloalkyl, cyclopropyl, cyclobutyl, —OR6, —NHC(═O)—C3-C6cycloalkyl, Cycle Q, —SF₅ or group Y

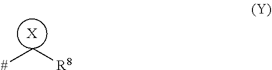

wherein X is a 3- or 4-membered carbocyclic ring and R8 is halogen, cyano, C1-C6alkyl, C1-C6haloalkyl or —C1-C6alkyl-CN. Group Y may be represented by the following structures:

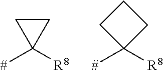

Preferably R2a is C1-C4alkyl, C1-C4haloalkyl, —OR6, SF₅ or group Y, wherein X is 3-membered carbocyclic ring and R8 is halogen, cyano, C1-C4alkyl, C1-C4haloalkyl or —C1-C4alkyl-CN, more preferably R2a is C1-C4alkyl, C1-C4haloalkyl, —OC1-C4alkyl, —OC1-C4haloalkyl, SF₅ or group Y, wherein X is 3-membered carbocyclic ring and R8 is halogen, cyano or halomethyl (e.g. trihalomethyl such as —CF₃). Specific examples of R2a include chloro, ethyl, propyl (e.g. isopropyl), butyl (e.g. tert-butyl), —CF₃, —O—CF₃, —NHC(═O)-cyclobutyl, —O—CH₂-cyclopropyl, phenyl, thiazol-2-yl, cyclopropyl, cyclobutyl, 1-CF₃-cyclopropyl, 1-cyano-cyclobutyl and —SF₅. 1-halomethyl-cyclopropyl, 1-cyanocyclopropyl, halomethyl, —O-halomethyl and SF₅ are preferred, in particular tert-butyl, 1-CF₃-cyclopropyl, —CF₃, —O—CF₃ and SF₅. In some embodiments R2a is —CF₃.

R2b is hydrogen, halogen (e.g. chloro or fluoro), methyl, —NH₂, halomethyl, —OCH₃ or —O-halomethyl, preferably hydrogen or —OCH₃. Specific examples include hydrogen, —CF₃, chloro, fluoro, —NH₂ and —OCH₃.

R3 is hydrogen, halogen, —C1-C6alkyl, —OC1-C6alkyl, —O—Cycle P, —O—Cycle Q1, —C1-C6alkyl-R9, —OC1-C6alkyl-R9, wherein in each alkyl group (e.g. C1-C6alkyl) or moiety (e.g. the alkyl moiety in —OC1-C6alkyl) in the foregoing one non-terminal —CH₂— (e.g. a —CH₂— other than the first or the last —CH₂— in the alkyl moiety within —OC1-C6alkyl) may be replaced by —NH— or —O— and wherein each alkyl group or moiety in the foregoing may be substituted by one or more halogen, wherein R9 is halogen, cyano, hydroxyl, —OC1-C2alkyl, —NH₂, —NH(C1-C2alkyl), —N(C1-C2alkyl)₂, —C(═O)NH₂, —C(═O)NH(C1-C2alkyl), —C(═O)N(C1-C2alkyl)₂, —C(═O)OH, —C(═O)O—C1-C2alkyl, —C(═O)—C1-C2alkyl, —NH(C═O)—C1-C2alkyl, —NH—S(O)₂—C1-C2alkyl, Cycle P or Cycle Q1, preferably R3 is hydrogen, halogen (e.g. chloro or fluoro), —OC1-C4alkyl, C1-C4alkyl, —OC1-C4alkyl-R9, —OC1-C2alkyl-NH—C1-C2alkyl-R9 or —O-Cycle P, wherein R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1, wherein Cycle P is a 5- to 6-membered heterocyclic ring optionally substituted by one to three R10 and Cycle Q1 is 5-6 membered heteroaryl optionally substituted by one to three R7a, wherein each R7a and R10 is methyl, more preferably R3 is hydrogen, halogen (e.g. fluoro or chloro), —OC1-C4alkyl, —OC1-C3alkyl-halogen (e.g. fluoro or chloro) or OC1-C3alkyl-OCH₃.

In some embodiments R3 is hydrogen, halogen, C1-C3alkyl (n-alkyl) or —OC1-C3alkyl (n-alkyl), preferably hydrogen, halogen (e.g. chloro or fluoro) or —OC1-C3alkyl (n-alkyl).

Specific examples of R3 include hydrogen, methyl, fluoro, methoxy, —O(tetrahydropyran-4-yl), —O(CH₂)₂(pyridin-2-yl), —O(CH₂)₂(morpholin-4-yl), —O(CH₂)₂NH(CH₂)₂OH, —O-(piperid-4-yl), —O-(tert-butyl), —OCH(CH₃)₂, —OCH₂(1-methylcyclopropyl), —OCH₂CH₂C(CH₃)₂OCH₃, —OCH₂CH₂F, —O—CH₂CH₂NH₂, —O—CH₂CH₂NH—C(═O)CH₃, —O—CH₂CH₂NH—SO₂—CH₃, —OCH₂CH₂OCH₃, —OCH₂CH₂OH, —O—CH₂C(═O)NH₂ and —O—CH₂C(═O)OH.

In some embodiments R1a and R3 together form a —CH₂—CH₂—CH₂— or a —CH₂—CH₂—CH₂—CH₂— moiety in which one of the —CH₂— units (i.e. in the —CH₂—CH₂—CH₂— or —CH₂—CH₂—CH₂—CH₂— moiety) is optionally replaced by —NH—; in other embodiments R1a and R3 together form a —CH₂—CH₂—CH₂— moiety.

R4 is —OH or —NH₂.

R6 is C1-C6alkyl, C1-C6haloalkyl or C1-C4alkylene-C3-C6cycloalkyl, preferably C1-C4alkyl or C1-C4haloalkyl, in particular methyl or halomethyl (e.g. trihalomethyl). Specific examples include —CF₃ and —CH₂-cyclopropyl.

Each R7 is independently C1-C4alkyl, preferably methyl.

Each R7a is independently C1-C4alkyl, preferably methyl.

R8 is halogen, cyano, C1-C6alkyl, C1-C6haloalkyl or —C1-C6alkyl-CN, preferably halogen, cyano, C1-C4alkyl, C1-C4haloalkyl or —C1-C4alkyl-CN, more preferably halogen (e.g. chloro or fluoro), cyano or halomethyl (e.g. trihalomethyl). Specific examples of R8 include —CF₃ and cyano.

R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1, preferably wherein Cycle P is a 5- to 6-membered heterocyclic ring optionally substituted by one to three R10 and Cycle Q1 is 5-6 membered heteroaryl optionally substituted by one to three R7a, more preferably wherein R9 is halogen (e.g. chloro or fluoro), cyano, hydroxyl, —OC1-C2alkyl, —NH₂, —C(═O)NH₂, —C(═O)OH, —NH(C═O)—C1-C2alkyl, —NH—S(O)₂—C1-C2alkyl, pyridinyl or morpholinyl. Specific examples of R9 include pyridin-2-yl, morpholin-4-yl, 1-methylcyclopropyl, —OCH₃, —F, —NH₂, —NH—C(═O)CH₃, —NH—SO₂—CH₃, —OH, —C(═O)NH₂ and —C(═O)OH.

Each R10 is independently C1-C4alkyl, preferably methyl.

Cycle P is a 3- to 6-membered carbocyclic ring or a 3- to 6-membered heterocyclic ring, each optionally substituted by one to three R10, preferably a 5- to 6-membered heterocyclic ring, e.g. containing one to two heteroatoms selected from nitrogen and oxygen, optionally substituted by one to three R10, preferably substituted by no more than one R10.

Cycle Q is phenyl or 5-6 membered heteroaryl, each optionally substituted by one to three R7, preferably optionally substituted by no more than one R7. The heteroaryl may contain one to three heteroatoms atoms, e.g. one to two heteroatoms, as ring members selected form nitrogen and sulfur, e.g. wherein no more than one heteroatom is sulfur. Specific examples include thiazolyl such as thiazol-2-yl.

Cycle Q1 is phenyl or 5-6 membered heteroaryl, each optionally substituted by one to three R7a, preferably a 5-6 membered heteroaryl, e.g. containing one to two nitrogen atoms, optionally substituted by one to three R7a, preferably substituted by no more than one R7a.

X is a 3- or 4-membered carbocyclic ring, preferably a 3-membered carbocyclic ring.

In some embodiments A1 is —C(R3)=, A2 is —N═, B1, B2, B3 and B4 are —CH═.

In some embodiments A1 is —C(R3)=, A2 is —CH═, B1, B2, B3 and B4 are —CH═.

In some embodiments A1 is —N═, A2 is —CH═, B1, B2, B3 and B4 are —CH═.

In some embodiments A1 is —N═, A2 is —N═, B1, B2, B3 and B4 are —CH═.

In some embodiments A1 is —N═, A2 is —N═, B1 and B2 are —N═ and B3 and B4 are —CH═.

In some embodiments A1 is —N═, A2 is —N═, B1 is —CH═, B2 is —N═ and B3 and B4 are —CH═.

In some embodiments A1 is —N═, A2 is —N═, B1, B2, and B4 are —CH═ and B3 is —C(R2b)=, wherein R2b is other than hydrogen.

In some embodiments A1 is —N═, A2 is —N═, B2, B3 and B4 are —CH═ and B1 is —C(R2b)=, wherein R2b is other than hydrogen.

In an embodiment (Embodiment A) the compound is a compound of formula I, wherein

A1 is —N═ or —C(R3)=;

A2 is —N═ or —CH═:

optionally at least one of A1 and A2 is —N═;

L is —NH—;

B1 and B2 are independently —N═ or —C(R2b)=;

B3 and B4 are independently —C(R2b)=; no more than one R2b on B1, B2, B3 and B4 is other than hydrogen;

R1a is hydrogen, halogen, C1-C3alkyl (n-alkyl), C1-C3alkyl(n-alkyl)-NH₂, C1-C3alkyl(n-alkyl)-OH, —NH₂, —NH(C1-C3alkyl (n-alkyl)), —NH(C(═O)—C1-C2alkyl), —OC1-C3alkyl (n-alkyl) or C1-C3haloalkyl (n-alkyl);

R1b is hydrogen, C1-C3alkyl (n-alkyl), —OC1-C3alkyl (n-alkyl) or —NH₂;

at least one of R1a and R1b is hydrogen or R1a and R1b together form a —CH═CH—CH═CH— moiety;

R2a is C1-C4alkyl, C1-C4haloalkyl, —OC1-C4alkyl, —OC1-C4haloalkyl, —SF₅ or group Y

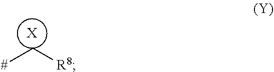

wherein X is 3-membered carbocyclic ring and R8 is halogen, cyano, C1-C4alkyl, C1-C4haloalkyl or —C1-C4alkyl-CN;

R2b is hydrogen or —OCH₃;

R3 is hydrogen, halogen, —OC1-C4alkyl, C1-C4alkyl, —OC1-C4alkyl-R9, —OC1-C2alkyl-NH—C1-C2alkyl-R9 or —O—Cycle P;

R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;

Cycle P is a 5- to 6-membered heterocyclic ring optionally substituted by one to three R10;

Cycle Q1 is 5-6 membered heteroaryl optionally substituted by one to three R7a; and each R7a and R10 is methyl.

In another embodiment (Embodiment Ai) the compound of formula (I) is as defined in Embodiment A wherein R3 is hydrogen, halogen or —OC1-C3alkyl (n-alkyl).

The above examples of substituent definitions and embodiments given for the compound of formula I may be combined with Embodiment A or Embodiment Ai in any combination where feasible.

In another embodiment (Embodiment B) the compound of formula (I) is a compound of formula (I-a)

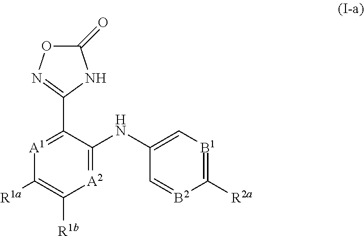

wherein

A1 is —N═ or —C(R3)=;

A2 is —N═ or —CH═;

B1 and B2 are independently —N═ or —C(R2b)= wherein no more than one R2b on B1 and B2 is other than hydrogen;

R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);

R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;

R1a and R1b may together form a —CH═CH—CH═CH— moiety;

R2a is halogen, C1-C6alkyl, C1-C6haloalkyl, cyclopropyl, cyclobutyl, —OR6, —NHC(═O)—C3-C6cycloalkyl, Cycle Q, —SF₅ or group Y

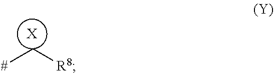

wherein X is a 3- or 4-membered carbocyclic ring and R8 is halogen, cyano, C1-C6alkyl, C1-C6haloalkyl or —C1-C6alkyl-CN;

R2b is hydrogen, halogen, methyl, —NH₂, halomethyl, —OCH₃ or —O-halomethyl;

R3 is hydrogen, halogen, C1-C6alkyl, —OC1-C6alkyl, —O—Cycle P, —O—Cycle Q1, —C1-C6alkyl-R9 or -OC1-C6alkyl-R9, wherein in each alkyl group or moiety in the foregoing one non-terminal —CH₂— may be replaced by —NH— or —O— and wherein each alkyl group or moiety in the foregoing may be substituted by one or more halogen, wherein R9 is halogen, cyano, hydroxyl, —OC1-C2alkyl, —NH₂, —NH(C1-C2alkyl), —N(C1-C2alkyl)₂, —C(═O)NH₂, —C(═O)NH(C1-C2alkyl), —C(═O)N(C1-C2alkyl)₂, —C(═O)OH, —C(═O)O—C1-C2alkyl, —C(═O)—C1-C2alkyl, —NH(C═O)—C1-C2alkyl, —NH—S(O)₂—C1-C2alkyl, Cycle P or Cycle Q1;

R1a and R3 may together form a —CH₂—CH₂—CH₂— or a —CH₂—CH₂—CH₂—CH₂— moiety in which one of the —CH₂— units is optionally replaced by —NH—;

R4 is —OH or —NH₂;

R6 is C1-C6alkyl, C1-C6haloalkyl or C1-C4alkylene-C3-C6cycloalkyl;

Cycle P is a 3- to 6-membered carbocyclic ring or a 3- to 6-membered heterocyclic ring, each optionally substituted by one to three R10;

Cycle Q is phenyl or 5-6 membered heteroaryl, each optionally substituted by one to three R7, preferably optionally substituted by no more than one R7, wherein the heteroaryl contains one to three heteroatoms atoms, e.g. one to two heteroatoms, as ring members selected form nitrogen and sulfur, e.g. wherein no more than one heteroatom is sulfur;

Cycle Q1 is phenyl or 5-6 membered heteroaryl, each optionally substituted by one to three R7a; and each R7, R7a and R10 is independently C1-C4alkyl.

In another embodiment (Embodiment Bi) the compound of formula (I) is a compound of formula (I-a) as defined in Embodiment B wherein R3 is hydrogen, halogen, C1-C3alkyl (n-alkyl) or —OC1-C3alkyl (n-alkyl).

The above examples of substituent definitions and embodiments given for the compound of formula (I) may be combined with Embodiment B and Embodiment Bi in any combination where feasible.

In another embodiment (Embodiment C) the compound of formula (I) is a compound of formula (I-a) wherein

A1 is —N═ or —C(R3)=;

A2 is —N═ or —CH═;

optionally at least one of A1 and A2 is —N═;

B1 and B2 are independently —N═,—or —C(R2b)= wherein no more than one R2b on B1 and B2 is other than hydrogen;

R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);

R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;

R1a and R1b may together form a —CH═CH—CH═CH— moiety;

R2a is C1-C4alkyl, C1-C4haloalkyl, —OC1-C4alkyl, —OC1-C4haloalkyl, SF₅ or group Y

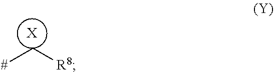

wherein X is 3-membered carbocyclic ring and R8 is halogen, cyano, C1-C4alkyl, C1-C4haloalkyl or —C1-C4alkyl-CN;

R2b is hydrogen, halogen, methyl, —NH₂, halomethyl, —OCH₃ or —O-halomethyl;

R3 is hydrogen, halogen, —OC1-C4alkyl, C1-C4alkyl, —OC1-C4alkyl-R9, —OC1-C2alkyl-NH—C1-C2alkyl-R9 or —O—Cycle P;

R4 is —OH or —NH₂.

R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;

Cycle P is a 5- to 6-membered heterocyclic ring optionally substituted by one to three R10;

Cycle Q1 is 5-6 membered heteroaryl optionally substituted by one to three R7a; and each R7a and R10 is methyl.

In another embodiment (Embodiment Ci) the compound of formula (I) is a compound of formula (I-a) as defined in Embodiment C wherein R3 is hydrogen, halogen or —OC1-C3alkyl (n-alkyl).

The above examples of substituent definitions and embodiments given for the compound of formula (I) may be combined with Embodiment C and Embodiment Ci in any combination where feasible.

In another embodiment (Embodiment D) the compound of formula I is a compound of formula (I-a) wherein

A1 is —N═ or —C(R3)=;

A2 is —N═ or —CH═;

optionally at least one of A1 and A2 is —N═;

B1 and B2 are independently —N═ or —C(R2b)=, wherein no more than one R2b on B1 and B2 is other than hydrogen;

R1a is hydrogen, halogen, C1-C3alkyl (n-alkyl), C1-C3alkyl(n-alkyl)-NH₂, C1-C3alkyl(n-alkyl)-OH, —NH₂, —NH(C1-C3alkyl (n-alkyl)), —NH(C(═O)—C1-C2alkyl), —OC1-C3alkyl (n-alkyl) or C1-C3haloalkyl (n-alkyl);

R1b is hydrogen, C1-C3alkyl (n-alkyl), —OC1-C3alkyl (n-alkyl) or —NH₂;

at least one of R1a and R1b is hydrogen or R1a and R1b together form a —CH═CH—CH═CH— moiety;

R2a is C1-C4alkyl, C1-C4haloalkyl, —OC1-C4alkyl, —OC1-C4haloalkyl, SF₅ or group Y

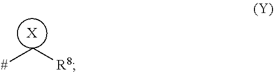

wherein X is 3-membered carbocyclic ring and R8 is halogen, cyano, C1-C4alkyl, C1-C4haloalkyl or —C1-C4alkyl-CN;

R2b is hydrogen or —OCH₃;

R3 is hydrogen, halogen, —OC1-C4alkyl, —C1-C4alkyl, —OC1-C4alkyl-R9, —OC1-C2alkyl-NH—C1-C2alkyl-R9 or —O—Cycle P;

R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;

Cycle P is a 5- to 6-membered heterocyclic ring optionally substituted by one to three R10;

Cycle Q1 is 5-6 membered heteroaryl optionally substituted by one to three R7a; each R7a and R10 is methyl.

In another embodiment (Embodiment Di) the compound of formula (I) is a compound of formula (I-a) as defined in Embodiment D wherein R3 is hydrogen, halogen or —OC1-C3alkyl (n-alkyl).

The above examples of substituent definitions and embodiments given for the compound of formula (I) may be combined with Embodiment D and Embodiment Di in any combination where feasible.

In another embodiment (Embodiment E) the compound of formula I is a compound of formula (I-a) wherein

A1 is —N═ or —C(R3)=;

A2 is —N═ or —CH═;

optionally at least one of A1 and A2 is —N═;

B1 and B2 are independently —N═ or —C(R2b)=, wherein no more than one R2b on B1 and B2 is other than hydrogen;

R1a is hydrogen, chloro, methyl or —NH₂;

R1b hydrogen, methyl, methoxy or —NH₂

at least one of R1a and R1b is hydrogen or R1a and R1b together form a —CH═CH—CH═CH— moiety;

R2a is C1-C4alkyl, C1-C4haloalkyl, —OC1-C4alkyl, —OC1-C4haloalkyl, SF₅ or group Y

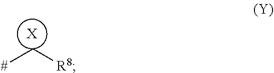

wherein X is 3-membered carbocyclic ring and R8 is halogen, cyano or halomethyl;

R2b is hydrogen or —OCH₃; and

R3 is hydrogen, halogen, —OC1-C4alkyl, —OC1-C3alkyl-halogen or —OC1-C3alkyl-OCH₃.

The above examples of substituent definitions and embodiments given for the compound of formula (I) may be combined with Embodiment E in any combination where feasible.

In another embodiment (Embodiment F) the compound of formula (I) may be the compound of formula (I-b)

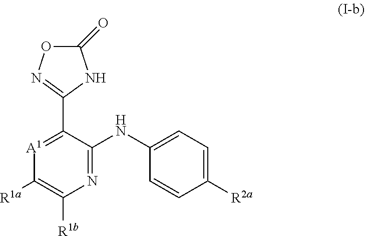

wherein

A1 is —N═ or —C(R3)=;

R1a is hydrogen, chloro, methyl or —NH₂;

R1b hydrogen, methyl, methoxy or —NH₂;

at least one of R1a and R1b is hydrogen;

R2a is tert-butyl, 1-(CF₃)cyclopropyl, —CF₃, —O—CF₃ or SF₅;

R3 is hydrogen, halogen (e.g. fluoro or chloro), —OC1-C4alkyl, —C1-C4alkyl, —OC1-C4alkyl-R9, —OC1-C2alkyl-NH—C1-C2alkyl-R9 or Cycle P;

R9 is halogen (e.g. fluoro or chloro), cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;

Cycle P is a 5- to 6-membered heterocyclic ring optionally substituted by one to three R10;

Cycle Q1 is 5-6 membered heteroaryl optionally substituted by one to three R7a; each R7a and R10 is methyl. 1-(CF₃)cyclopropyl means group Y

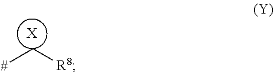

wherein X is cyclopropyl and R8 is CF₃.

1-(cyano)cyclobutyl means group Y

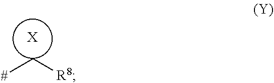

wherein X is cyclobutyl and R8 is CN.

The above examples of substituent definitions and embodiments given for the compound of formula (I) may be combined with Embodiment F in any combination where feasible.

In another embodiment (Embodiment G) the compound of formula (I) may be the compound of formula (I-b) wherein

A1 is —N═ or —C(R3)=;

R1a is hydrogen, chloro, methyl or —NH₂;

R1b hydrogen, methyl, methoxy or —NH₂;

at least one of R1a and R1b is hydrogen;

R2a is tert-butyl, 1-(CF₃)cyclopropyl, —CF₃, —O—CF₃ or SF₅;

R3 is hydrogen, halogen (e.g. fluoro or chloro), —OC1-C4alkyl, —OC1-C3alkyl-halogen (e.g. fluoro or chloro) or —OC1-C3alkyl-OCH₃.

The above examples of substituent definitions and embodiments given for the compound of formula (I) may be combined with Embodiment G in any combination where feasible.

In another embodiment (Embodiment H) the compound is a compound of formula I, wherein

A1 is —N═ or —C(R3)=;

A2 is —N═ or —CH═;

optionally at least one of A1 and A2 is —N═;

B1 and B2 are independently —N═- or —C(R2b)=;

B3 is —C(R2b)=(e.g. —CH═);

B4 is —CH═;

wherein no more than one R2b on B1, B2 and B3 is other than hydrogen;

R1a is hydrogen, chloro, methyl, ethyl, trifluoromethyl, methoxy, —CH₂CH₂—NH₂, —CH₂—OH, —NH—CH₃, —NH₂ or —NH—C(═O)—CH₃;

R1b is hydrogen, methyl, methoxy or —NH₂;

at least one of R1a and R1b is hydrogen or R1a and R1b together form a —CH═CH—CH═CH— moiety or R1a and R3 together form a —CH₂—CH₂—CH₂— or a —CH₂—CH₂—CH₂—CH₂— moiety in which one of the —CH₂— units is optionally replaced by —NH—;

R2a is chloro, ethyl, isopropyl, tert-butyl, trifluoromethyl, —O—CF₃, —NHC(═O)-cyclobutyl, —O—CH₂— cyclopropyl, phenyl, thiazol-2-yl, cyclopropyl, cyclobutyl, 1-(CF₃)cyclopropyl, 1(cyano)cyclobutyl or —SF₅;

R2b is hydrogen, fluoro, chloro, —NH₂, —CF₃ or —OCH₃; and

R3 is hydrogen, methyl, fluoro, methoxy, —O(tetrahydropyran-4-yl), —O(CH₂)₂(pyridin-2-yl), —O(CH₂)₂(morpholin-4-yl), —O(CH₂)₂NH(CH₂)₂OH, —O-(piperid-4-yl), —O-(tert-butyl), —OCH(CH₃)₂, —OCH₂(1-methylcyclopropyl), —OCH₂CH₂C(CH₃)₂OCH₃, —OCH₂CH₂F, —O—CH₂CH₂NH₂, —O—CH₂CH₂NH—C(═O)CH₃, —O—CH₂CH₂NH—SO₂—CH₃, —OCH₂CH₂OCH₃, —OCH₂CH₂OH, —O—CH₂C(═O)NH₂ or —O—CH₂C(═O)OH.

In another embodiment (Embodiment I) the compound of formula I may be the compound of formula (I-c)

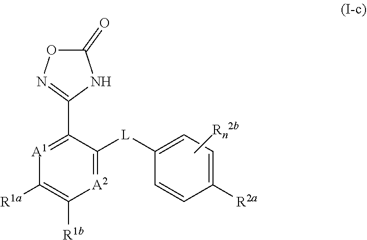

wherein

A1 is —N═ or —C(R3)-;

A2 is —N═;

L is —NH—;

R1a is hydrogen, C1-C3alkyl (n-alkyl) optionally substituted with one R4, or R1a is —NH₂, —NHR5, —N(R5)₂ or —OC1-C3alkyl (n-alkyl);

R1b is hydrogen;

R2a is halogen, C1-C4alkyl, C1-C4haloalkyl, —OC1-C4alkyl or —OC1-C4haloalkyl;

R2b is halogen;

R3 is hydrogen;

R4 is —OH or —NH₂;

each R5 is independently C1-C3alkyl (n-alkyl); and

n is 0 or 1.

In Embodiment I the following examples of substituent definitions and embodiments may be combined in any combination.

A1 is —N═ or —C(R3)-.

A2 is —N═.

L is —NH—.

R1a is hydrogen, C1-C3alkyl (n-alkyl) optionally substituted with one R4, or R1a is —NH₂, —NHR5, —N(R5)₂ or —OC1-C3alkyl (n-alkyl), preferably —CH₃, —CH₂CH₃, —NH₂, —NHCH₃ or —OCH₃.

R1b is hydrogen;

R2a is halogen, C1-C4alkyl, C1-C4haloalkyl, —OC1-C4alkyl or —OC1-C4haloalkyl, preferably —CF₃, Cl, Br, —OCH₃ or —OCF₃.

R2b is halogen.

R3 is hydrogen.

R4 is —OH or —NH₂.

each R5 is independently C1-C3alkyl (n-alkyl), e.g. —CH₃, —CH₂CH₃ or —CH₂CH₂CH₃. n is 0 or 1, preferably 0.

In one embodiment A1 is —N═.

In one embodiment A1 is —C(R3)-.

In one embodiment A1 is —N═ or —C(R3)-; A2 is —N═; L is —NH—; R1a is hydrogen, —CH₃, —CH₂CH₃, —NH₂, —NHCH₃ or —OCH₃; R1b is hydrogen; R2a is —CF₃, Cl, Br, —OCH₃, —OCF₃; R3 is hydrogen; n is 0.

In further embodiments the invention provides the following compounds and pharmaceutically acceptable salts thereof:
3-[2-[4-(trifluoromethyl)anilino]-3-pyridyl]-4H-1,2,4-oxadiazol-5-one;
3-[3-[4-(trifluoromethyl)anilino]pyrazin-2-yl]-4H-1,2,4-oxadiazol-5-one;
3-[3-[4-(trifluoromethoxy)anilino]pyrazin-2-yl]-4H-1,2,4-oxadiazol-5-one;
3-[6-amino-3-[4-(trifluoromethyl)anilino]pyrazin-2-yl]-4H-1,2,4-oxadiazol-5-one;
3-[6-methoxy-3-[4-(trifluoromethyl)anilino]pyrazin-2-yl]-4H-1,2,4-oxadiazol-5-one;
3-[6-(hydroxymethyl)-3-[4-(trifluoromethyl)anilino]pyrazin-2-yl]-4H-1,2,4-oxadiazol-5-one;
3-[6-(2-aminoethyl)-3-[4-(trifluoromethyl)anilino]pyrazin-2-yl]-4H-1,2,4-oxadiazol-5-one;
3-[3-[2-chloro-4-(trifluoromethoxy)anilino]pyrazin-2-yl]-4H-1,2,4-oxadiazol-5-one;
3-[3-[4-(trifluoromethyl)anilino]-2-pyridyl]-4H-1,2,4-oxadiazol-5-one;
3-[5-(trifluoromethyl)-2-[4-(trifluoromethyl)anilino]-3-pyridyl]-4H-1,2,4-oxadiazol-5-one;
3-[4-methoxy-2-[4-(trifluoromethyl)anilino]-3-pyridyl]-4H-1,2,4-oxadiazol-5-one;
3-[5-methoxy-3-[4-(trifluoromethyl)anilino]pyrazin-2-yl]-4H-1,2,4-oxadiazol-5-one;
3-[4-fluoro-2-[4-(trifluoromethyl)anilino]-3-pyridyl]-4H-1,2,4-oxadiazol-5-one;
3-[3-[4-(cyclopropylmethoxy)anilino]pyrazin-2-yl]-4H-1,2,4-oxadiazol-5-one;
3-[3-(4-tert-butylanilino)pyrazin-2-yl]-4H-1,2,4-oxadiazol-5-one;
3-[3-(4-isopropylanilino)pyrazin-2-yl]-4H-1,2,4-oxadiazol-5-one;
3-[3-(4-ethylanilino)pyrazin-2-yl]-4H-1,2,4-oxadiazol-5-one;
3-[3-(4-thiazol-2-ylanilino)pyrazin-2-yl]-4H-1,2,4-oxadiazol-5-one;
3-[3-[3-fluoro-4-(trifluoromethyl)anilino]pyrazin-2-yl]-4H-1,2,4-oxadiazol-5-one;
3-[3-[3-chloro-4-(trifluoromethyl)anilino]pyrazin-2-yl]-4H-1,2,4-oxadiazol-5-one;
3-[3-[2-methoxy-4-(trifluoromethyl)anilino]pyrazin-2-yl]-4H-1,2,4-oxadiazol-5-one;
3-[3-[3-methoxy-4-(trifluoromethyl)anilino]pyrazin-2-yl]-4H-1,2,4-oxadiazol-5-one;
3-[3-[[2-(trifluoromethyl)pyrimidin-5-yl]amino]pyrazin-2-yl]-4H-1,2,4-oxadiazol-5-one;
3-[3-[[6-(trifluoromethyl)-3-pyridyl]amino]pyrazin-2-yl]-4H-1,2,4-oxadiazol-5-one;
3-[3-[[2-(trifluoromethyl)pyrimidin-5-yl]amino]pyrazin-2-yl]-4H-1,2,4-oxadiazol-5-one;
3-[4,6-dimethyl-2-[4-(trifluoromethyl)anilino]-3-pyridyl]-4H-1,2,4-oxadiazol-5-one;
N-[4-[[3-(5-oxo-4H-1,2,4-oxadiazol-3-yl)pyrazin-2-yl]amino]phenyl]cyclobutanecarboxamide;
3-[4-methyl-2-[4-(trifluoromethyl)anilino]-3-pyridyl]-4H-1,2,4-oxadiazol-5-one;
3-[3-(4-phenylanilino)pyrazin-2-yl]-4H-1,2,4-oxadiazol-5-one;
3-[3-[4-chloro-3-(trifluoromethyl)anilino]pyrazin-2-yl]-4H-1,2,4-oxadiazol-5-one;
3-[5-methyl-3-[4-(trifluoromethyl)anilino]pyrazin-2-yl]-4H-1,2,4-oxadiazol-5-one;
3-[6-methyl-3-[4-(trifluoromethyl)anilino]pyrazin-2-yl]-4H-1,2,4-oxadiazol-5-one;
3-[3-[3-amino-4-(trifluoromethyl)anilino]pyrazin-2-yl]-4H-1,2,4-oxadiazol-5-one;
3-[3-[2-amino-4-(trifluoromethyl)anilino]pyrazin-2-yl]-4H-1,2,4-oxadiazol-5-one;
N-[6-(5-oxo-4H-1,2,4-oxadiazol-3-yl)-5-[4-(trifluoromethyl)anilino]pyrazin-2-yl]acetamide;
3-[5-amino-3-[4-(trifluoromethyl)anilino]pyrazin-2-yl]-4H-1,2,4-oxadiazol-5-one;
3-[3-[4-(trifluoromethyl)anilino]quinoxalin-2-yl]-4H-1,2,4-oxadiazol-5-one.
3-[2-[4-(trifluoromethyl)anilino]phenyl]-4H-1,2,4-oxadiazol-5-one;
3-[3-(4-cyclopropylanilino)pyrazin-2-yl]-4H-1,2,4-oxadiazol-5-one;
3-[3-[4-[1-(trifluoromethyl)cyclopropyl]anilino]pyrazin-2-yl]-4H-1,2,4-oxadiazol-5-one;
3-[3-(4-cyclobutylanilino)pyrazin-2-yl]-4H-1,2,4-oxadiazol-5-one;
1-[4-[[3-(5-oxo-4H-1,2,4-oxadiazol-3-yl)pyrazin-2-yl]amino]phenyl]cyclobutanecarbonitrile;
3-[3-(4-chloroanilino)pyrazin-2-yl]-4H-1,2,4-oxadiazol-5-one;
3-[5-chloro-2-[4-(trifluoromethyl)anilino]-3-pyridyl]-4H-1,2,4-oxadiazol-5-one;
3-[5-amino-2-[4-(trifluoromethyl)anilino]-3-pyridyl]-4H-1,2,4-oxadiazol-5-one;
3-[5-(methylamino)-2-[4-(trifluoromethyl)anilino]-3-pyridyl]-4H-1,2,4-oxadiazol-5-one;
3-[5,6-dimethyl-2-[4-(trifluoromethyl)anilino]-3-pyridyl]-4H-1,2,4-oxadiazol-5-one;
3-[2-(4-chloroanilino)-4-(2-hydroxyethoxy)-3-pyridyl]-4H-1,2,4-oxadiazol-5-one;
3-[3-[4-(trifluoromethyl)anilino]-6,7-dihydro-5H-cyclopenta[c]pyridin-4-yl]-4H-1,2,4-oxadiazol-5-one;
3-[3-[4-(pentafluoro-λ6-sulfanyl)anilino]pyrazin-2-yl]-4H-1,2,4-oxadiazol-5-one;
3-[4-(2-hydroxyethoxy)-2-[4-(trifluoromethyl)anilino]-3-pyridyl]-4H-1,2,4-oxadiazol-5-one;
3-[4-isopropoxy-2-[4-(trifluoromethyl)anilino]-3-pyridyl]-4H-1,2,4-oxadiazol-5-one;
3-[4-tert-butoxy-2-[4-(trifluoromethyl)anilino]-3-pyridyl]-4H-1,2,4-oxadiazol-5-one;
3-[4-[2-(2-hydroxyethylamino)ethoxy]-2-[4-(trifluoromethyl)anilino]-3-pyridyl]-4H-1,2,4-oxadiazol-5-one;
3-[4-(2-aminoethoxy)-2-[4-(trifluoromethyl)anilino]-3-pyridyl]-4H-1,2,4-oxadiazol-5-one;
N-[2-[[3-(5-oxo-4H-1,2,4-oxadiazol-3-yl)-2-[4-(trifluoromethyl)anilino]-4-pyridyl]oxy]ethyl]acetamide;
N-[2-[[3-(5-oxo-4H-1,2,4-oxadiazol-3-yl)-2-[4-(trifluoromethyl)anilino]-4-pyridyl]oxy]ethyl]methanesulfonamide;
2-[[3-(5-oxo-4H-1,2,4-oxadiazol-3-yl)-2-[4-(trifluoromethyl)anilino]-4-pyridyl]oxy]acetic acid;
2-[[3-(5-oxo-4H-1,2,4-oxadiazol-3-yl)-2-[4-(trifluoromethyl)anilino]-4-pyridyl]oxy]acetamide;
3-[4-(2-morpholinoethoxy)-2-[4-(trifluoromethyl)anilino]-3-pyridyl]-4H-1,2,4-oxadiazol-5-one;
3-[4-[2-(2-pyridyl)ethoxy]-2-[4-(trifluoromethyl)anilino]-3-pyridyl]-4H-1,2,4-oxadiazol-5-one;
3-[4-(2-methoxyethoxy)-2-[4-(trifluoromethyl)anilino]-3-pyridyl]-4H-1,2,4-oxadiazol-5-one;
3-[4-[(1-methylcyclopropyl)methoxy]-2-[4-(trifluoromethyl)anilino]-3-pyridyl]-4H-1,2,4-oxadiazol-5-one;
3-[4-tetrahydropyran-4-yloxy-2-[4-(trifluoromethyl)anilino]-3-pyridyl]-4H-1,2,4-oxadiazol-5-one;
3-[4-(2-fluoroethoxy)-2-[4-(trifluoromethyl)anilino]-3-pyridyl]-4H-1,2,4-oxadiazol-5-one;
3-[4-(3-methoxy-3-methyl-butoxy)-2-[4-(trifluoromethyl)anilino]-3-pyridyl]-4H-1,2,4-oxadiazol-5-one;
3-[4-(4-piperidyloxy)-2-[4-(trifluoromethyl)anilino]-3-pyridyl]-4H-1,2,4-oxadiazol-5-one.

Some intermediates useful for the preparation of compounds of formula (I) are new and form further aspects of the invention. Accordingly, in a further aspect the invention provides compounds of formula (Int-I)

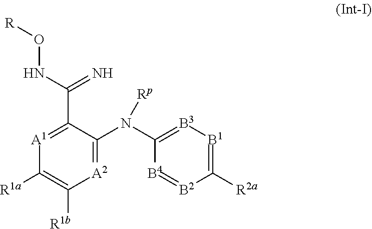

wherein A1, A2, B1, B2, B3, B4, R1a, R1b and R2a are as defined for compounds of formula (I), including as defined in preferred definitions embodiments thereof (e.g. as defined in Embodiment A, Embodiment B, Embodiment Bi, Embodiment C, Embodiment D, Embodiment Di, Embodiment E, Embodiment F or Embodiment G), and wherein when R1a, R1b and R2a include an amine moiety, the amine moiety may be protected by a protecting group such as tert-butyl carbamate (Boc), 9-Fluorenylmethylcarbamate (Fmoc), benzyl carbamate, acetamide, trifluoroacetamide, phthalimide, benzylamine, tritylamine, benzylideneamine or p-Toluenesulfonamide, preferably tert-butyl carbamate (Boc);
wherein R is hydrogen or —C(═O)—O—C1-C4alkyl, wherein the alkyl is optionally substituted with 1 to 3 halogen (e.g. —C(═O)—O—CH₂—CF₃), preferably —C(═O)—O—CH₃ or —C(═O)—O-phenyl, wherein the phenyl is optionally substituted with an NO₂ group, and

R^{p} is hydrogen or a protecting group such as tert-butyl carbamate (Boc), 9-Fluorenylmethylcarbamate (Fmoc), benzyl carbamate, acetamide, trifluoroacetamide, phthalimide, benzylamine, tritylamine, benzylideneamine or p-Toluenesulfonamide, preferably tert-butyl carbamate (Boc); and wherein the compound of formula (Int-I) is not the following compound: Benzenecarboximidamide, N-hydroxy-2-[(4-methylphenyl)amino]- (CAS 57076-14-9).

In another embodiment (Embodiment Int-Ia) the compound of formula (Int-I) is a compound of formula (Int-I) wherein

A1 is —N═ or —C(R3)=;

A2 is —N═ or —CH═;

L is —NH—;

B1 and B2 are independently —N═ or —C(R2b)=;

B3 and B4 are independently —C(R2b)=;

no more than one R2b on B1, B2, B3 and B4 is other than hydrogen;

R1a is hydrogen, halogen, C1-C3alkyl (n-alkyl) optionally substituted with one R4, or R1a is —NH₂—NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);

R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;

R1a and R1b may together form a —CH═CH—CH═CH— moiety in which one or two non-adjacent CH are optionally replaced by N;

R2a is halogen, C1-C6alkyl, C1-C6haloalkyl, —OR6, —NHC(═O)—C3-C6cycloalkyl or Cycle Q;

R2b is hydrogen, halogen, methyl, —NH₂, halomethyl or —OCH₃, —O-halomethyl;

R3 is hydrogen, halogen, C1-C3alkyl (n-alkyl) or —OC1-C3alkyl (n-alkyl);

R4 is —OH or —NH₂;

R6 is C1-C6alkyl, C1-C6haloalkyl or C1-C4alkylene-C3-C6cycloalkyl;

Cycle Q is phenyl or 5-6 membered heteroaryl, each optionally substituted by one to three R7;

each R7 is independently C1-C4alkyl;

R and R^{p} are as defined above;

and wherein when R1a, R1b and R2a include an amine moiety, the amine moiety may be protected by a protecting group such as tert-butyl carbamate (Boc), 9-Fluorenylmethylcarbamate (Fmoc), benzyl carbamate, acetamide, trifluoroacetamide, phthalimide, benzylamine, tritylamine, benzylideneamine or p-Toluenesulfonamide, preferably tert-butyl carbamate (Boc);
and wherein the compound of formula (Int-I) is not the following compound: Benzenecarboximidamide, N-hydroxy-2-[(4-methylphenyl)amino]- (CAS 57076-14-9).

In particular in some embodiments of compounds of formula (Int-I) A1 is —N═ and A2 is —N═.

In some embodiments of compounds of formula (Int-I) A1 is —N═ and A2 is —CH═; or

In some embodiments of compounds of formula (Int-I) A1 is —C(R3)= and A2 is —N═.

In a further aspect the invention provides compounds of formula (Int-II)

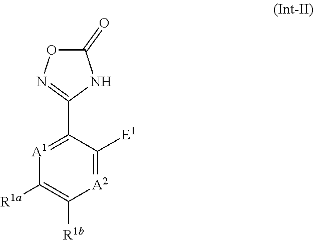

Wherein A1, A2, R1a and R1b are as defined for compounds of formula (I), including as defined in preferred definitions embodiments thereof (e.g. as defined in Embodiment A, Embodiment B, Embodiment Bi, Embodiment C, Embodiment D, Embodiment Di, Embodiment E, Embodiment F or Embodiment G), and wherein E1 is halogen, e.g. chloro, bromo or iodo, preferably chloro or bromo, or a leaving group selected from a perfluoroalkylsulfonate such as triflate and a sulfonic acid ester such as tosylate or mesylate and wherein the compound is not the following compounds:
1,2,4-Oxadiazol-5(2H)-one, 3-(3-chloro-2-quinoxalinyl)- (CAS 95893-50-8);
1,2,4-Oxadiazol-5(2H)-one, 3-(3-chloro-2-pyridinyl)- (CAS 1696580-83-2);
1,2,4-Oxadiazol-5(2H)-one, 3-(2-bromo-6-chlorophenyl)- (CAS 2297731-08-7);
1,2,4-Oxadiazol-5(2H)-one, 3-(2,5-dibromophenyl)- (CAS 2289845-71-0);
1,2,4-Oxadiazol-5(2H)-one, 3-(2-iodophenyl)- (CAS 2284537-41-1);
1,2,4-Oxadiazol-5(2H)-one, 3-(2-chloro-6-fluorophenyl)- (CAS 2248668-96-2);
1,2,4-Oxadiazol-5(2H)-one, 3-(2-chloro-4-methylphenyl)- (CAS 2003326-44-9)
1,2,4-Oxadiazol-5(2H)-one, 3-(2-bromo-4-methoxyphenyl)- (CAS 1880198-26-4);
1,2,4-Oxadiazol-5(2H)-one, 3-(2-bromo-4,5-difluorophenyl)- (CAS 1870368-87-8);
1,2,4-Oxadiazol-5(2H)-one, 3-(5-bromo-2-chlorophenyl)- (1702990-97-3);
1,2,4-Oxadiazol-5(2H)-one, 3-(2,5-dichlorophenyl)- (CAS 1701787-66-7);
1,2,4-Oxadiazol-5(2H)-one, 3-(2-bromo-5-fluorophenyl)- (CAS 1699277-36-5);
1,2,4-Oxadiazol-5(2H)-one, 3-(2-bromo-4-fluorophenyl)- (CAS 1694114-82-3);
1,2,4-Oxadiazol-5(2H)-one, 3-(2-bromo-5-methoxyphenyl)- (CAS 1693707-16-2);
1,2,4-Oxadiazol-5(2H)-one, 3-(4-bromo-2-chlorophenyl)- (CAS 1592806-60-4);
1,2,4-Oxadiazol-5(2H)-one, 3-(2-chloro-6-methoxyphenyl)- (CAS 1564718-39-3);
1,2,4-Oxadiazol-5(2H)-one, 3-(2-bromo-4-methylphenyl)- (CAS 1554525-50-6);
1,2,4-Oxadiazol-5(2H)-one, 3-(2-bromo-4-chlorophenyl)- (CAS 1516828-53-7);
1,2,4-Oxadiazol-5(2H)-one, 3-(2-bromo-5-chlorophenyl)- (CAS 1484262-05-6);
1,2,4-Oxadiazol-5(2H)-one, 3-(2-chloro-4-fluorophenyl)- (CAS 1343167-41-8);
1,2,4-Oxadiazol-5(2H)-one, 3-(4-amino-2,5-difluorophenyl)- (CAS 1269523-71-8);
1,2,4-Oxadiazol-5(2H)-one, 3-(4-amino-2,6-difluorophenyl)- (CAS 1228776-09-7);
1,2,4-Oxadiazol-5(2H)-one, 3-(4-amino-2-fluorophenyl)- (CAS 1228776-07-5);
1,2,4-Oxadiazol-5(2H)-one, 3-(4-amino-2-chlorophenyl)- (CAS 1228775-82-3);
1,2,4-Oxadiazol-5(2H)-one, 3-(2,4-dichlorophenyl)- (CAS 1184540-06-4);
1,2,4-Oxadiazol-5(2H)-one, 3-(2-bromophenyl)- (CAS 1183944-55-9);
1,2,4-Oxadiazol-5(2H)-one, 3-(2-bromo-4,5-dimethoxyphenyl)- (CAS 1183565-19-6);
1,2,4-Oxadiazol-5(2H)-one, 3-(2,6-difluoro-3-methoxyphenyl)- (CAS 1138333-23-9);
1,2,4-Oxadiazol-5(2H)-one, 3-(2,6-dichlorophenyl)- (CAS 91774-85-5);
1,2,4-Oxadiazol-5(2H)-one, 3-(2-chlorophenyl)- (CAS 16672-15-4);
1,2,4-Oxadiazol-5(2H)-one, 3-(2-fluorophenyl)- (CAS 16672-12-1);

In an embodiment (Embodiment Int-IIa) the compound of formula (Int-I) is a compound of formula (Int-I) wherein

A1 is —N═ or —C(R3)=;

A2 is —N═ or —CH═;

R1a is hydrogen, halogen, C1-C3alkyl (n-alkyl) optionally substituted with one R4, or R1a is —NH₂—NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);

R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;

R1a and R1b may together form a —CH═CH—CH═CH— moiety in which one or two non-adjacent CH are optionally replaced by N;

R4 is —OH or —NH₂;

and wherein E1 is as defined above.

In particular, in compounds of formula (Int-II) preferably at least one of A1 and A2 is —N═.

In some embodiments of compounds of formula (Int-II) A1 is —N═.

In some embodiments of compounds of formula (Int-II) A1 is —C(R3)=.

In some embodiments of compounds of formula (Int-II) A2 is —N═.

In some embodiments of compounds of formula (Int-II) A2 is —CH═.

In some embodiments of compounds of formula (Int-II) A1 is —N═ and A2 is —N═.

In some embodiments of compounds of formula (Int-II) A1 is —N═ and A2 is —CH═.

In some embodiments of compounds of formula (Int-II) A1 is —C(R3)= and A2 is —N═.

In some embodiments of compounds of formula (Int-II) A1 is —C(R3)= and A2 is —CH═.

In particular, compounds of formula (Int-II) preferably at least one of A1 and A2 is —N═ and E1 is halogen, e.g. chloro, bromo or iodo, preferably chloro or bromo.

In some embodiments of compounds of formula (Int-II) A1 is —N═ and E1 is halogen, e.g. chloro, bromo or iodo, preferably chloro or bromo.

In some embodiments of compounds of formula (Int-II) A1 is —C(R3)= and E1 is halogen, e.g. chloro, bromo or iodo, preferably chloro or bromo.

In some embodiments of compounds of formula (Int-II) A2 is —N═ and E1 is halogen, e.g. chloro, bromo or iodo, preferably chloro or bromo.

In some embodiments of compounds of formula (Int-II) A2 is —CH═ and E1 is halogen, e.g. chloro, bromo or iodo, preferably chloro or bromo.

In some embodiments of compounds of formula (Int-II) A1 is —N═ and A2 is —N═ and E1 is halogen, e.g. chloro, bromo or iodo, preferably chloro or bromo.

In some embodiments of compounds of formula (Int-II) A1 is —N═ and A2 is —CH═ and E1 is halogen, e.g. chloro, bromo or iodo, preferably chloro or bromo.

In some embodiments of compounds of formula (Int-II) A1 is —C(R3)= and A2 is —N═ and E1 is halogen, e.g. chloro, bromo or iodo, preferably chloro or bromo.

In some embodiments of compounds of formula (Int-II) A1 is —C(R3)= and A2 is —CH═ and E1 is halogen, e.g. chloro, bromo or iodo, preferably chloro or bromo.

In some embodiments of compounds of formula (Int-II) Aland A2 are —N═;

R1a is hydrogen, halogen, C1-C3alkyl (n-alkyl) optionally substituted with one R4, or R1a is —NH₂—NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl); and

R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂.

The present invention relates also to pharmaceutical compositions that comprise a compound of formula (I) as active ingredient or a pharmaceutically acceptable salt thereof, which can be used especially in the treatment of neoplastic diseases, in particular cancer, as described herein. Compositions may be formulated for non-parenteral administration, such as nasal, buccal, rectal, pulmonary, vaginal, sublingual, topical, transdermal, ophthalmic, or, especially, for oral administration, e.g. in the form of oral solid dosage forms, e.g. granules, pellets, powders, tablets, film or sugar-coated tablets, effervescent tablets, hard and soft gelatin or hydroxypropylmethylcellulose (HPMC) capsules, coated as applicable, orally disintegrating tablets, oral solutions, lipid emulsions or suspensions, or for parenteral administration, such as intravenous, intramuscular, or subcutaneous, intrathecal, intradermal or epidural administration, to mammals, especially humans, e.g. in the form of solutions, lipid emulsions or suspensions containing microparticles or nanoparticles. The compositions may comprise the active ingredient alone or, preferably, together with a pharmaceutically acceptable carrier.

The compounds of formula (I) or pharmaceutically acceptable salts thereof can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar-coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc.

Suitable excipients for parenteral formulations are e.g. water, alcohols, polyols, glycerol, vegetable oils, lecithin, surfactants etc.

Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain other therapeutically valuable substances.

The dosage can vary within wide limits and will, of course, be fitted to the individual requirements in each particular case. In general, in the case of oral administration a daily dosage of about 1 to 1000 mg per person of a compound of general formula (I) should be appropriate, although the above lower or upper limit can also be exceeded when necessary.

The compounds of formula (I) can also be used in combination with one or more other pharmaceutically active compounds, which are either effective against the same disease, preferably using a different mode of action, or which reduce or prevent possible undesired side effects of the compounds of formula (I). The combination partners can be administered in such a treatment either simultaneously, e.g. by incorporating them into a single pharmaceutical formulation, or consecutively by administration of two or more different dosage forms, each containing one or more than one of the combination partners.

Compounds of formula (I) according to the invention as described above or pharmaceutically acceptable salts thereof are particularly useful for the treatment of neoplastic diseases such as cancer, in particular carcinoma, sarcoma, leukemia, myeloma and lymphoma and cancers of the brain and spinal cord, e.g. when administered in therapeutically effective amounts. In some embodiments, the cancer to be treated by the compounds of the present invention is mediated by modulation of the interaction of YAP/TAZ with TEAD. In some embodiments the compounds of the invention may treat the cancer by modulating the interaction between YAP/TAZ and TEAD. In some embodiments the compounds of the invention may inhibit the interaction between YAP/TAZ and TEAD, In some embodiments, the cancer is a solid tumour. In some embodiments, the cancer is a hematologic malignancy. In some instances, the solid tumour is a sarcoma or carcinoma. In some embodiments, the solid tumour is a sarcoma. In some instances, the solid tumour is a carcinoma.

Examples of such proliferation disorders and diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial-like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous and neuroendocrine neoplasms, meningiomas, nerve sheath tumours, granular cell tumours and alveolar soft part sarcomas, Hodgkin's and non-Hodgkin's lymphomas, B-cell lymphoma, T-cell lymphoma, hairy-cell lymphoma, Burkitts lymphoma and other lymphoreticular neoplasms, plasma cell tumours, mast cell tumours, immunoproliferative diseases, leukemias, miscellaneous myeloproliferative disorders, lymphoproliferative disorders and myelodysplastic syndromes.

Examples of cancers in terms of the organs and parts of the body affected include, but are not limited to, the breast, cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach (gastric cancer), intestine, pancreas, bone marrow, hematological malignancies (such as lymphoma, leukemia, myeloma or lymphoid malignancies), bladder, urinary tract, kidneys, skin, thyroid, brain, head, neck, prostate and testis. Preferably the cancer is selected from the group consisting of breast cancer, prostate cancer, cervical cancer, ovarian cancer, gastric cancer, colorectal cancer, pancreatic cancer, liver cancer, brain cancer, neuroendocrine cancer, lung cancer, kidney cancer, bladder cancer, mesothelioma, hematological malignancies, melanomas and sarcomas.

The term “treatment” or “treating” as used herein in the context of treating a disease or disorder, pertains generally to treatment and therapy, whether of a human or an animal (e.g., in veterinary applications), in which some desired therapeutic effect is achieved, for example, the inhibition of the progress of the disease or disorder, and includes a reduction in the rate of progress, a halt in the rate of progress, alleviation of symptoms of the disease or disorder, amelioration of the disease or disorder, and cure of the disease or disorder. Treatment as a prophylactic measure (i.e., prophylaxis) is also included. For example, use with patients who have not yet developed the disease or disorder, but who are at risk of developing the disease or disorder, is encompassed by the term “treatment.” For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

The term “pharmaceutical composition” is defined herein to refer to a solid or liquid formulation containing at least one therapeutic agent to be administered to a subject, e.g., a mammal or human, with one or more pharmaceutically acceptable excipients, in order to prevent or treat a particular disease or condition affecting the mammal.

The term “pharmaceutically acceptable” as used herein refers to items such as compounds and salts thereof, materials, compositions and/or dosage forms, which are, within the scope of sound medical judgment, suitable for contact with the tissues of a warm-blooded animal, e.g., a mammal or human, without excessive toxicity or other complications commensurate with a reasonable benefit/risk ratio. The compounds of formula (I) can be synthesized by methods given below, by methods given in the experimental part below or by analogous methods. The schemes described herein are not intended to present an exhaustive list of methods for preparing the compounds of formula (I); rather, additional techniques of which the skilled chemist is aware may be also used for the compound synthesis.

It is understood by one skilled in the art of organic synthesis that optimum reaction conditions may vary with the particular reactants or solvents used, but such conditions can be determined by routine optimization procedures. In some cases, the order of performing the following reaction schemes, and/or reaction steps, may be varied to facilitate the reaction or to avoid the formation of unwanted side products. In addition, the functionality present at various positions of the molecule must be compatible with the reagents and reactions proposed. Such restrictions to the substituents, which are compatible with the reaction conditions, will be readily apparent to one skilled in the art and alternate methods must then be used. Furthermore in some of the reactions mentioned herein it may be necessary or desirable to protect any sensitive groups in compounds and it will be assumed that such protecting groups (PG) as necessary are in place. Conventional protecting groups may be used in accordance with standard practice, well known in the art (for illustration see Greene T. W, Wuts P. G. M, Protective Groups in Organic Synthesis, 5th Edition, Publisher: John Wiley & Sons, 2014). The protecting groups may be removed at any convenient stage in the synthesis using conventional techniques well known in the art, or they may be removed during a later reaction step or work-up.

In the general sequence of reactions outlined below, the abbreviations A1, A2, B1, B2, B3, B4 and the generic groups, R1a, R1b, R2a and R2b are as defined for formula (I), unless otherwise specified. Other abbreviations used herein are explicitly defined, or are as defined in the experimental section. In addition, the skilled person will understand that general sequence of reactions outlined below is applicable to all tautomeric forms even if only one tautomer form is drawn.

The necessary starting materials for the synthetic methods as described herein, if not commercially available, may be made by procedures which are described in the scientific literature, or may be made from commercially available compounds using adaptations of processes reported in the scientific literature. The reader is further referred to March J., Smith M., Advanced Organic Chemistry, 7th Edition, Publisher: John Wiley & Sons, 2013 for general guidance on reaction conditions and reagents.

The compounds according to the present invention, pharmaceutically acceptable salts, solvates, and hydrates thereof can be prepared according to the general sequence of reactions outlined below, followed, if necessary, by:

manipulation of substituents to give a new final product. These manipulations may include, but are not limited to, reduction, oxidation, alkylation, acylation, substitution, coupling including transition-metal catalyzed coupling and hydrolysis reactions which are commonly known by those skilled in the art; removing any protecting groups;

forming a pharmaceutically acceptable salt; or

forming a pharmaceutically acceptable solvate or hydrate.

Generally, compounds of formula (I), wherein L is —NH—, can be obtained by the coupling reaction of a compound of formula (5) and a compound of formula (6), wherein E3 and E4 are leaving groups, such as chlorine, imidazole, phenol, 4-nitrophenol, 2,2,2-trifluoro-ethanol, methanol, ethanol or 1-hydroxypyrrolidine-2,5-dione, followed by intra-molecular cyclization (Scheme 1).

The coupling reaction and the cyclization reaction can be performed sequentially but are generally simultaneously performed in situ. Depending on the reactivity of compound of formula (6), different reaction conditions can be applied, which would be readily apparent for a skilled chemist. For example, when a compound of formula (6) is phosgene or more frequently a phosgene analogue (such as bis(trichloromethyl) carbonate or trichloromethyl chloroformate), the reaction is typically performed in aprotic and inert solvents such as dichloromethane, chloroform, acetonitrile, tetrahydrofuran, ethyl acetate (more frequently dichloromethane) in presence or absence of a base such as triethylamine, 4-(dimethylamino)pyridine or N,N-diisopropylethylamine. Reactions are typically run from −40° C. to 50° C., generally 0° C.

When a compound of formula (6) is 1,1′-carbonyldiimidazole (which can be activated by methylation prior to the reaction), methyl chloroformate, phenyl chloroformate, 4-nitrophenyl chloroformate, 2,2,2-trifluoroethyl chloroformate or N,N′-disuccinimidyl carbonate, the reaction can be performed in presence or absence of a base, such as sodium hydride, triethylamine, pyridine (diluted or neat), 4-(dimethylamino)pyridine, or 1,8-diazabicyclo[5.4.0]undec-7-ene in aprotic solvents such as dichloromethane, chloroform, acetonitrile, tetrahydrofuran, ethyl acetate or N,N-dimethylformamide.

Reactions are typically run from −10° C. to 150° C. More frequently, compounds of formula (I) are prepared from the reaction between a compound of formula (5) and 1,1′-carbonyldiimidazole in N,N-dimethylformamide at a temperature from 100° C. to 120° C.

Alternatively, when a compound of formula (6) is dimethylcarbonate, the reaction is typically performed in aprotic solvents such as N,N-dimethylformamide or dimethyl sulfoxide in the presence of an inorganic base such as sodium hydroxide, sodium carbonate or an organic base such as triethylamine or pyridine. Reactions are generally run from −10° C. to 120° C., more frequently at room temperature.

Compounds of formula (5) are generally obtained from a condensation reaction between a compound of formula (4) and a hydroxylamine salt (generally hydroxylamine hydrochloride). The reaction is typically run in alcoholic solvents such as methanol, ethanol, iso-propanol or tert-butanol (more frequently iso-propanol) in a presence or absence of an inorganic base such as sodium carbonate or more frequently sodium hydrogen carbonate or in the presence or absence of an organic base such as sodium tert-butoxide, triethylamine, pyridine or alike at a temperature ranging from 20° C. to 90° C.

Compounds of formula (4) can be generated from a compound of formula (2), wherein E1 is a halogen or a leaving group such as a triflate, and a compound of formula (3), wherein E2 is an amino group, via a transition-metal catalyst reaction coupling. Typical catalysts include palladium(II) acetate, tris(dibenzylideneacetone)dipalladium(0) or alike. The reaction is typically run at a temperature from 0° C. to 150° C., more frequently from 80° C. to 110° C. Usually the reaction is performed in the presence of a ligand such as di-tert-butyl-[3,6-dimethoxy-2-(2,4,6-triisopropylphenyl)phenyl]phosphane, di-tert-butyl-[2,3,4,5-tetramethyl-6-(2,4,6-triisopropylphenyl)phenyl]phosphane, 2-(dicyclohexylphosphino)biphenyl, 4,5-bis(diphenylphospheno)-9,9-dimethylxanthene or the like and a base such as sodium tert-butylate, cesium carbonate, potassium carbonate, more frequently cesium carbonate in a large variety of inert solvents such as toluene, tetrahydrofuran, dioxane, 1,2-dichloroethane, N,N-dimethylformamide, dimethylsulfoxide, water and acetonitrile, or a mixture of solvents, more frequently in dioxane.

Alternatively, compounds of formula (4) can be prepared from a compound of formula (2), wherein E1 is an amino group and a compound of formula (3), wherein E2 is a boronic acid, via a Chan-Lam coupling reaction. The Chan-Lam coupling is typically performed in the presence of a copper(II) catalyst such as copper(II) acetate and under oxygen atmosphere. The reaction can be run in inert organic solvent such dichloromethane, chloroform or tetrahydrofuran (more frequently dichloromethane) in the presence of a base such triethylamine, pyridine or alike (more frequently triethylamine) at a temperature ranging from 0° C. to 70° C. (generally room temperature).

Compounds of formula (2) and compounds of formula (3) can be obtained from commercial sources, or are prepared following procedures described in literature, or by procedures known by a person skilled in the art.

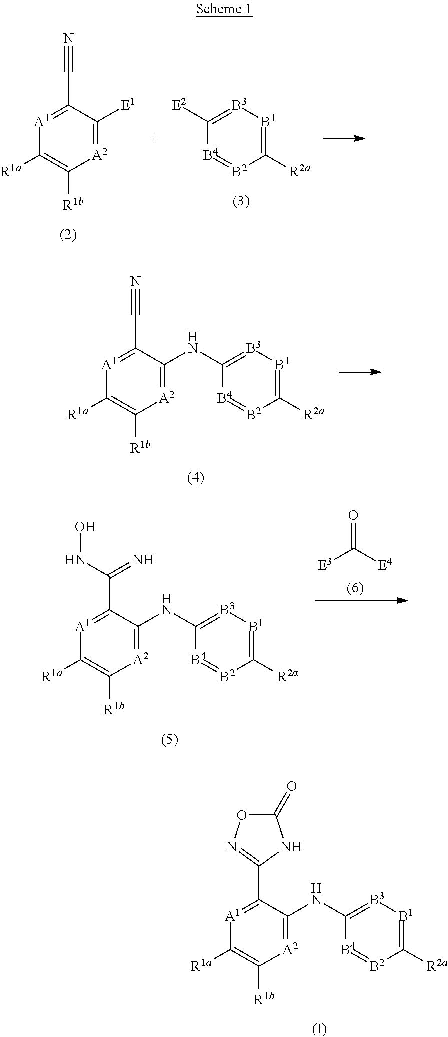

Alternatively, compounds of formula (I), wherein L is —NH—, can be obtained by nucleophilic aromatic substitution reaction of a compound of formula (8), wherein E1 is a halogen or a leaving group such as a triflate, and a compound of formula (3), wherein E2 is an amino group (Scheme 2).

The nucleophilic aromatic substitution can be performed in a large variety of solvents such as methanol, ethanol, iso-propanol, N,N-dimethylformamide, dimethyl sulfoxide or tetrahydrofuran and in presence of an organic base, such as triethylamine, N,N-Diisopropylethylamine, pyridine or an inorganic base such as sodium hydroxide, potassium bicarbonate or sodium hydrogen carbonate. Reactions are typically run from 50 to 150° C., using classical heating devices or microwave device. More frequently, the reaction is performed in iso-propanol at a temperature of 130° C. using microwave device.

Compounds of formula (8) can be prepared from a compound of formula (6), wherein E3 and E4 are leaving groups, such as chlorine, imidazole, phenol, 4-nitrophenol, 2,2,2-trifluoro-ethanol, methanol, ethanol or 1-hydroxypyrrolidine-2,5-dione and a compound of formula (7) using similar reaction conditions previously described in scheme 1 for a compound of formula (1).

Compounds of formula (7) are generally obtained from a condensation reaction between a compound of formula (2) and a hydroxylamine salt (generally hydroxylamine hydrochloride) using similar reaction conditions previously described in scheme 1 for a compound of formula (5).

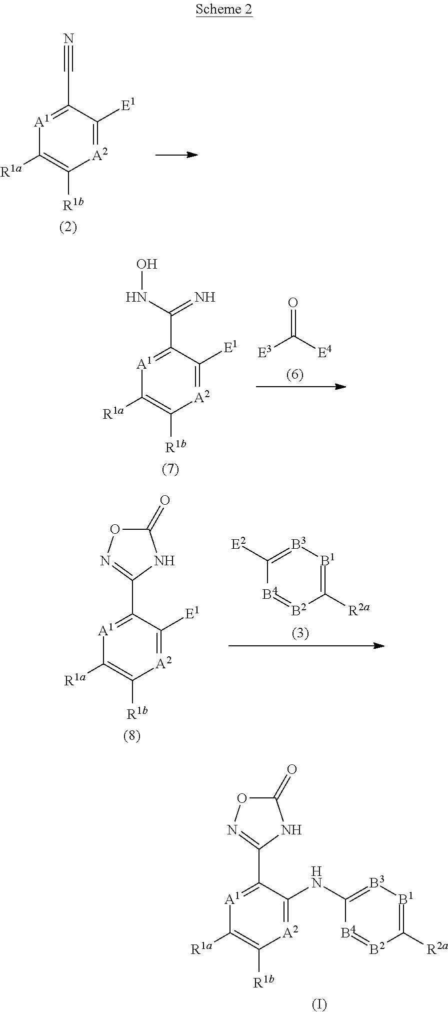

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and, as will be understood by the person skilled in the art, should not be construed as limiting the invention in any way.

### DESCRIPTION OF THE FIGURES

FIG. 1

FIG. 1 shows the result of an experiment comparing the dose-dependent efficacy and tolerability of the compound of Example 2 versus vehicle control in mice bearing NCI-H226 mesothelioma/squamous cell lung cancer cells.

The compound of Example 2 was administered orally daily at 50, 125 or 250 mg/kg, respectively. The upper panel (FIG. 1A) shows mean tumor volumes and the lower panel (FIG. 1B) shows mean body weight changes. Data points represent mean values+/−SEM (n=8 animals). Statistical analyses of the results were performed using the One-Way-ANOVA (Tukey test).

FIG. 2

FIG. 2 depicts the effects of the compound of Example 2 treatment on the TEAD-dependent PD marker CTGF.

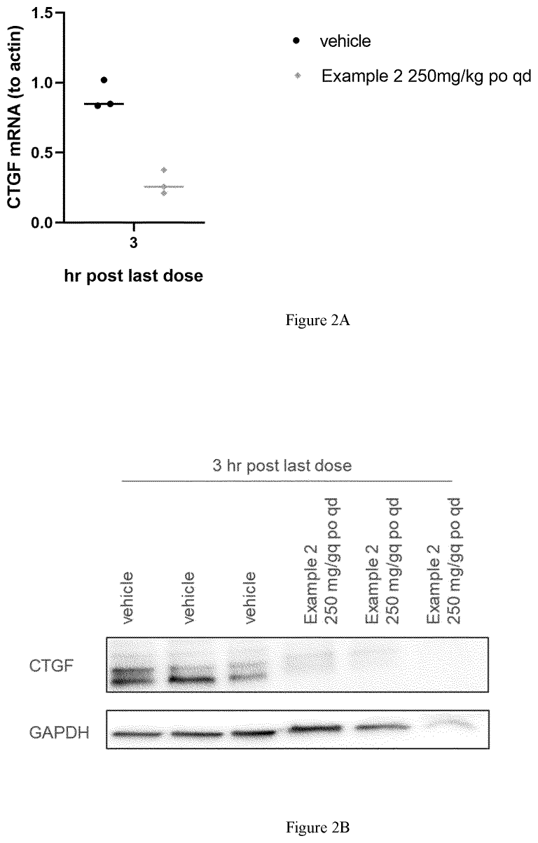

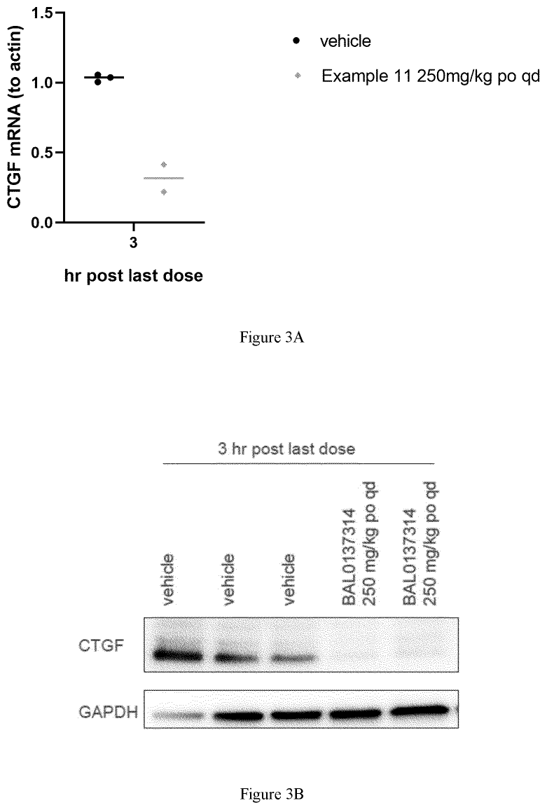

Animals were treated with the compound of Example 2 at 250 mg/kg po qd for 8 days and tumors were isolated from both vehicle and the compound of Example 2-treated groups 3 hours after the last dose was given. RNA and protein extracts from the tumors were prepared and assessed. The upper panel (FIG. 2A) shows CTGF mRNA levels that were normalized to actin in the respective animal tumors. Data points show the relative amount of CTGF mRNA, with each point representing an individual tumor. The line is drawn at the median. The lower panel (FIG. 2B) depicts the total levels of CTGF protein in the vehicle and compound-treated mice, with each lane representing an individual tumor. GAPDH was used as a loading control.

FIG. 3

FIG. 3 depicts the effects of treatment with the compound of Example 11 on the TEAD-dependent PD marker CTGF.

Animals were treated with the compound of Example 11 at 250 mg/kg po qd for 5 days and tumors were isolated from both vehicle and treated groups 3 hours after the last dose was given. RNA and protein extracts from the tumors were prepared and assessed. The upper panel (FIG. 3A) shows CTGF mRNA levels that were normalized to actin in the respective animal tumors. Data points show the relative amount of CTGF mRNA, with each point representing an individual tumor. The line is drawn at the median. The lower panel (FIG. 3B) depicts the total levels of CTGF protein in the vehicle and compound-treated mice, with each lane representing an individual tumor. GAPDH was used as a loading control.

### EXAMPLES

Preparation of Examples

All reagents and solvents are generally used as received from the commercial supplier; reactions are routinely performed with anhydrous solvents in well-dried glassware under nitrogen atmosphere, unless otherwise specified;

evaporations are carried out by rotary evaporation under reduced pressure and work-up procedures are carried out after removal of residual solids by filtration;

all temperatures are given in degree Celsius (° C.) and are approximate temperatures; unless otherwise noted, operations are carried out at room temperature (rt), that is typically in the range 18° C.-25° C.; column chromatography (by the flash procedure) is used to purify compounds and is performed using Merck silica gel 60 (70-230 mesh ASTM) unless otherwise stated;

classical flash chromatography is often replaced by automated systems. This does not change the separation process per se. A person skilled in the art will be able to replace a classical flash chromatography process by an automated one, and vice versa. Typical automated systems can be used, as they are provided by Büchi or Isco (combiflash) for instance;

reaction mixture can often be separated by preparative HPLC using water and acetonitrile as system of eluents, unless otherwise stated. A person skilled in the art will find suitable conditions for each separation; in some cases the compounds are isolated after purification in a form of the corresponding trifluoroacetic acid (TFA) salt (*1), or the respective formic acid salt (*2); such compounds are marked accordingly;

reactions, which required higher temperature, are usually performed using classical heating instruments; but can also be performed using microwave apparatus (CEM Explorer) at a power of 250 W, unless otherwise noted;

hydrogenation or hydrogenolysis reactions can be performed using hydrogen gas in balloon or using Parr-apparatus system or other suitable hydrogenation equipment;

concentration of solutions and drying of solids are performed under reduced pressure unless otherwise stated;

in general, the course of reactions is followed by TLC, HPLC, or LC/MS and reaction times are given for illustration only; yields are given for illustration only and are not necessarily the maximum attainable; the structure of the final products of the invention is generally confirmed by NMR and mass spectral techniques.

Proton NMR spectra are recorded on a Brucker 400 MHz spectrometer. Chemical shifts (δ) are reported in ppm relative to Me₄Si as internal standard, and NMR coupling constants (J values) are in Hertz (Hz). Each peak is denoted as a broad singlet (br), singlet (s), doublet (d), triplet (t), quadruplet (q), doublet of doublets (dd), triplet of doublets (td) or multiplet (m). Mass spectra are generated using a q-Tof Ultima (Waters AG or Thermo Scientific MSQ Plus) mass spectrometer in the positive or negative ESI mode.

The system is equipped with the standard Lockspray interface;

each intermediate is purified to the standard required for the subsequent stage and is characterized in sufficient detail to confirm that the assigned structure is correct;

analytical and preparative HPLC on non-chiral phases are performed using RP-C18 based columns;

the following abbreviations may be used (reference can also be made to The Journal of Organic Chemistry Guidelines for Authors, 2017 for a comprehensive list of standard abbreviations):
Ac Acetyl
ACN Acetonitrile
(BOC)₂ODi-tert-butyl dicarbonate
BOC tert-butoxy carbonyl group
BTC Bis(trichloromethyl)carbonate
Cat. no. Catalog number
CDCl₃ Deuterated chloroform
CDI 1,1′-Carbonyldiimidazole
DBU 1,8-Diazabicyclo[5.4.0]undec-7-ene
DCE 1,2-dichloroethane
DCM Dichloromethane
DIPEA N,N-Diisopropylethylamine
DMAP 4-Dimethylaminopyridine
DMC Dimethylcarbonate
DMF Dimethylformamide
DMSO Dimethyl sulfoxide
DMSO-d6 Deuterated dimethyl sulfoxide
DPPF 1,1′-Bis(diphenylphosphino)ferrocene
EA Ethyl acetate
ELSD Evaporative light scattering detection
EtOH Ethanol
Ex. Example
HATU 2-(7-Aza-1H-benzotriazole-1-yl)-1,1,3,3-tetramethyluronium hexafluorophosphate
c-Hex Cyclohexane
n-Hex n-Hexane
i-PrOH Iso-propanol
LAH Lithium aluminum hydride
LC/MS Liquid chromatography coupled to mass spectroscopy
Me₄Si Tetramethylsilane
MCI Mitsubishi gel with high porous polymer for reverse phase column chromatography
MeOH Methanol
MsCl Methanesulfonyl chloride
nt Not Tested
PBS Phosphate-Buffered Saline
PCR Polymerase Chain Reaction
Pd(PPh₃)₄ Tetrakis(triphenylphosphine)palladium(0)
Pd(OAc)₂ Palladium (II) acetate
PE Petroleum Ether
Pd₂dba₃ Tris(dibenzylideneacetone)dipalladium(0)
po per os
Py Pyridine
qd quaque die
RNA ribonucleic acid
SEM Standard Error of the Measurement
TBS tert-butyldimethylsilyl
t-BuBrettPhos 2-(di-t-butylphosphino)-3,6-dimethoxy-2′,4′,6′-tri-i-propyl-1,1′-biphenyl
t-BuOH tert-butanol
TEA Triethylamine
TEMPO 2,2,6,6-Tetramethylpiperidine 1-oxyl
TFAA Trifluoroacetic anhydride
THF Tetrahydrofuran
Tol Toluene
W Watt
XantPhos 4,5-bis(diphenylphospheno)-9,9-dimethylxanthene
X-Phos 2-Dicyclohexylphosphino-2′,4′,6′-triisopropylbiphenyl

The following Examples refer to the compounds of formula (I) as indicated in Table 1.

The Examples listed in the following table can be prepared using procedures described above, and detailed synthesis methodology is described in detail below. The Example numbers used in the leftmost column are used in the application text for identifying the respective compounds.

TABLE 1
Exemplified compounds

| | | Refer- | Reference | | |
|---|---|---|---|---|---|
| | | ence | for Prep- | ¹H-NMR | MS m/z |
| Ex. | Formula | Scheme | aration | (400 MHz) δ ppm | (+ESI) |
| 1 | 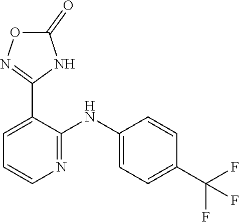 [SMILES: O=c1[nH]c(-c2cccnc2Nc2ccc(C(F)(F)F)cc2)no1] | 1 | — | DMSO-d₆: 13.09 (br, 1H), 9.31 (s, 1H), 8.46 (dd, J = 4.8, 1.8 Hz, 1H), 8.06 (dd, J = 7.8, 1.8 Hz, 1H), 7.92 (d, J = 8.5 Hz, 2H), 7.68 (d, J = 8.5 Hz, 2H), 7.12 (dd, J = 7.8, 4.8 Hz, 1H). | 323.1 [M + H]⁺ |
| 2 | 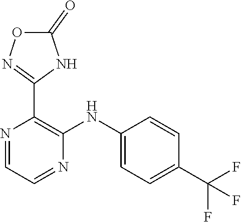 [SMILES: O=c1[nH]c(-c2nccnc2Nc2ccc(C(F)(F)F)cc2)no1] | 1 | Ex 1 (step 1 - 2) | DMSO-d₆: 13.40 (br, 1H), 9.25 (s, 1H), 8.50 (d, J = 2.4 Hz, 1H), 8.31 (d, J = 2.4 Hz, 1H), 7.91 (d, J = 8.5 Hz, 2H), 7.71 (d, J = 8.5 Hz, 2H). | 324.1 [M + H]⁺ |
| 3 | 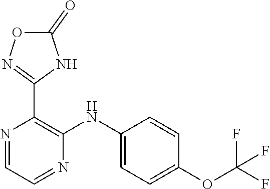 [SMILES: O=c1[nH]c(-c2nccnc2Nc2ccc(OC(F)(F)F)cc2)no1] | 1 | Ex. 1 (step 1 - 2), Ex. 2 (step 3) | DMSO-d₆: 13.69 (br, 1H), 9.02 (s, 1H), 8.44 (d, J = 2.4 Hz, 1H), 8.26 (d, J = 2.4 Hz, 1H), 7.78 (d, J = 8.6 Hz, 2H), 7.37 (d, J = 8.6 Hz, 2H). | 340.1 [M + H]⁺ |
| 4 | 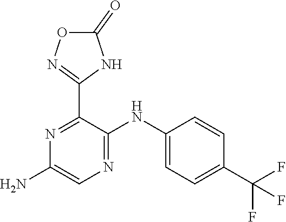 [SMILES: Nc1cnc(Nc2ccc(C(F)(F)F)cc2)c(-c2noc(=O)[nH]2)n1] | 1 | Ex. 1 (step 1 - 2), Ex. 2 (step 3) | DMSO-d₆: 12.96 (br, 1H), 8.73 (s, 1H), 7.94 (s, 1H), 7.58 (m, 4H), 6.18 (s, 2H). | 339.2 [M + H]⁺ |
| 5 | 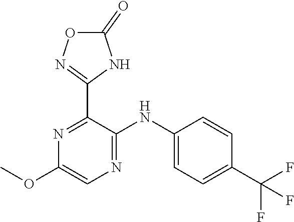 [SMILES: COc1cnc(Nc2ccc(C(F)(F)F)cc2)c(-c2noc(=O)[nH]2)n1] | 1 | Ex. 1 (step 2), Ex. 2 (step 3) | DMSO-d₆: 13.17 (br, 1H), 8.85 (s, 1H), 8.29 (s, 1H), 7.75 (d, J = 8.8 Hz, 2H), 7.64 (d, J = 8.8 Hz, 2H), 3.99 (s, 3H). | 354.1 [M + H]⁺ |
| 6 | 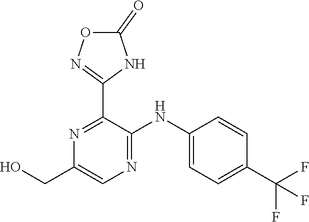 [SMILES: O=c1[nH]c(-c2nc(CO)cnc2Nc2ccc(C(F)(F)F)cc2)no1] | 1 | Ex. 1 (step 2), Ex. 2 (step 3) | DMSO-d₆: 13.27 (br, 1H), 9.18 (s, 1H), 8.53 (s, 1H), 7.90 (d, J = 8.8 Hz, 2H), 7.71 (d, J = 8.8 Hz, 2H), 5.47 (br, 1H), 4.64 (s, 2H). | 354.1 [M + H]⁺ |
| 7 | 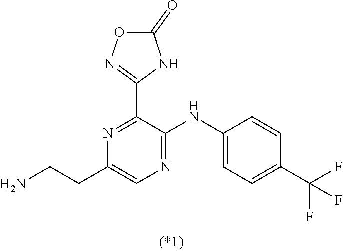 [SMILES: NCCc1cnc(Nc2ccc(C(F)(F)F)cc2)c(-c2noc(=O)[nH]2)n1] | 1 | Ex. 1 (step 2), Ex. 2 (step 3), Ex. 5 (step 6) | DMSO-d₆ + D₂O: 8.39 (s, 1H), 7.86 (d, J = 8.8 Hz, 2H), 7.69 (d, J = 8.8 Hz, 2H), 3.31 (t, J = 6.8 Hz, 2H), 3.07 (t, J = 6.8 Hz, 2H). | 367.2 [M + H]⁺ |
| 8 | 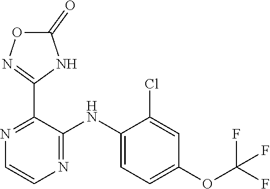 [SMILES: O=c1[nH]c(-c2nccnc2Nc2ccc(OC(F)(F)F)cc2Cl)no1] | 1 | Ex. 1 (step 1 - 2), Ex. 2 (step 3) | DMSO-d₆: 13.44 (br, 1H), 9.32 (s, 1H), 8.51-8.48 (m, 2H), 8.33 (d, J = 2.4 Hz, 1H), 7.72 (d, J = 2.8 Hz, 1H), 7.46 (dd, J = 2.8 and 8.8 Hz, 1H). | 374.1, 376.0 [M + H]⁺ |
| 9 | 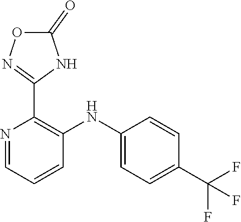 [SMILES: O=c1[nH]c(-c2ncccc2Nc2ccc(C(F)(F)F)cc2)no1] | 1 | Ex. 1 (step 1 - 2) | DMSO-d₆: 13.06 (br, 1H), 8.44 (s, 1H), 8.34 (dd, J = 4.4, 1.2 Hz, 1H), 7.96 (dd, J = 8.4, 1.2 Hz, 1H), 7.65 (d, J = 8.6 Hz, 2H), 7.54 (dd, J = 8.4, 4.4 Hz, 1H), 7.36 (d, J = 8.6 Hz, 2H). | 323.2 [M + H]⁺ |
| 10 | 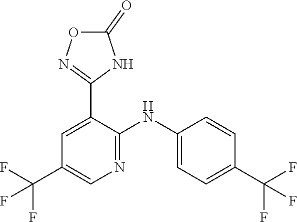 [SMILES: O=c1[nH]c(-c2cc(C(F)(F)F)cnc2Nc2ccc(C(F)(F)F)cc2)no1] | 1 | Ex. 1 (step 1 - 2), Ex. 2 (step 3) | DMSO-d₆: 13.21 (br, 1H), 9.63 (s, 1H), 8.78 (d, J = 2.0 Hz, 1H), 8.41 (d, J = 2.0 Hz, 1H), 7.91 (d, J = 8.6 Hz, 2H), 7.72 (d, J = 8.6 Hz, 2H). | 391.1 [M + H]⁺ |
| 11 | 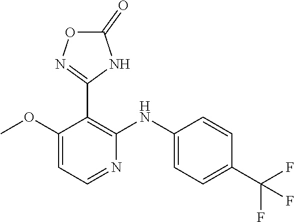 [SMILES: COc1ccnc(Nc2ccc(C(F)(F)F)cc2)c1-c1noc(=O)[nH]1] | 1 | Ex. 1 (step 1 - 3) | DMSO-d₆: 12.38 (br, 1H), 8.93 (s, 1H), 8.31 (d, J = 5.8 Hz, 1H), 7.82 (d, J = 8.6 Hz, 2H), 7.61 (d, J = 8.6 Hz, 2H), 6.83 (d, J = 5.8 Hz, 1H), 3.90 (s, 3H). | 353.1 [M + H]⁺ |
| 12 | 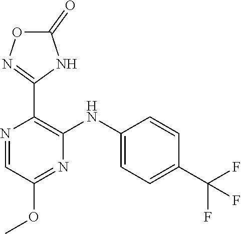 [SMILES: COc1cnc(-c2noc(=O)[nH]2)c(Nc2ccc(C(F)(F)F)cc2)n1] | 1 | Ex. 1 (step 1 - 2), Ex. 9 (step 3) | DMSO-d₆: 13.16 (br, 1H), 9.22 (s, 1H), 7.94 (s, 1H), 7.92 (d, J = 8.6 Hz, 2H), 7.72 (d, J = 8.6 Hz, 2H), 4.00 (s, 3H). | 354.1 [M + H]⁺ |
| 13 | 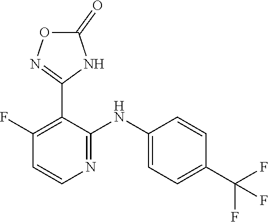 [SMILES: O=c1[nH]c(-c2c(F)ccnc2Nc2ccc(C(F)(F)F)cc2)no1] | 1 | Ex. 1 (step 1 - 3) | DMSO-d₆: 12.81 (br, 1H), 9.25 (s, 1H), 8.44 (dd, J = 9.0, 2.0 Hz, 1H), 7.83 (d, J = 8.8 Hz, 2H), 7.66 (d, J = 8.8 Hz, 2H), 7.05 (dd, J = 9.0, 2.0 Hz, 1H). | 341.1 [M + H]⁺ |
| 14 | 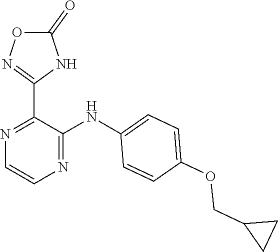 [SMILES: O=c1[nH]c(-c2nccnc2Nc2ccc(OCC3CC3)cc2)no1] | 2 | Ex. 1 (step 2) | DMSO-d₆: 13.29 (br, 1H), 8.72 (s, 1H), 8.36 (d, J = 2.2 Hz, 1H), 8.15 (d, J = 2.2 Hz, 1H), 7.50 (d, J = 9.0 Hz, 2H), 6.93 (d, J = 9.0 Hz, 2H), 3.80 (d, J = 6.8 Hz, 2H), 1.26-1.19 (m, 1H), 0.59-0.54 (m, 2H), 0.33-0.30 (m, 2H). | 326.1 [M + H]⁺ |
| 15 | 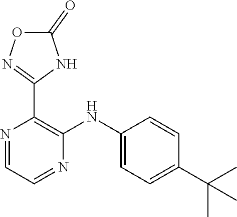 [SMILES: CC(C)(C)c1ccc(Nc2nccnc2-c2noc(=O)[nH]2)cc1] | 2 | Ex. 14 (step 3) | DMSO-d₆: 13.32 (br, 1H), 8.86 (s, 1H), 8.40 (d, J = 2.2 Hz, 1H), 8.19 (d, J = 2.2 Hz, 1H), 7.55 (d, J = 8.8 Hz, 2H), 7.39 (d, J = 8.8 Hz, 2H), 1.29 (s, 9H). | 312.2 [M + H]⁺ |
| 16 | 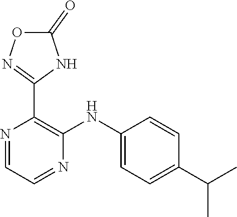 [SMILES: CC(C)c1ccc(Nc2nccnc2-c2noc(=O)[nH]2)cc1] | 2 | Ex. 14 (step 3) | DMSO-d₆: 13.32 (br, 1H), 8.85 (s, 1H), 8.40 (d, J = 2.2 Hz, 1H), 8.18 (d, J = 2.2 Hz, 1H), 7.54 (d, J = 8.4 Hz, 2H), 7.24 (d, J = 8.4 Hz, 2H), 2.90-2.85 (m, 1H), 1.20 (d, J = 7.2 Hz, 6H). | 298.2 [M + H]⁺ |
| 17 | 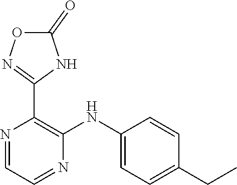 [SMILES: CCc1ccc(Nc2nccnc2-c2noc(=O)[nH]2)cc1] | 2 | Ex. 14 (step 3) | DMSO-d₆: 13.33 (br, 1H), 8.86 (s, 1H), 8.40 (d, J = 2.4 Hz, 1H), 8.18 (d, J = 2.4 Hz, 1H), 7.54 (d, J = 8.4 Hz, 2H), 7.21 (d, J = 8.4 Hz, 2H), 2.59 (q, J = 7.6 Hz, 2H), 1.18 (t, J = 7.6 Hz, 3H). | 284.1 [M + H]⁺ |
| 18 | 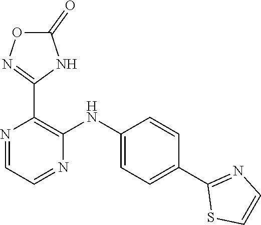 [SMILES: O=c1[nH]c(-c2nccnc2Nc2ccc(-c3nccs3)cc2)no1] | 1 | Ex. 1 (step 1 - 2) | DMSO-d₆: 13.39 (br, 1H), 9.14 (s, 1H), 8.50 (d, J = 2.4 Hz, 1H), 8.28 (d, J = 2.4 Hz, 1H), 7.96 (d, J = 8.6 Hz, 2H), 7.89 (d, J = 3.0 Hz, 1H), 7.84 (d, J = 8.6 Hz, 2H), 7.73 (d, J = 3.0 Hz, 1H). | 339.2 [M + H]⁺ |
| 19 | 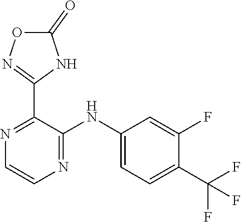 [SMILES: O=c1[nH]c(-c2nccnc2Nc2ccc(C(F)(F)F)c(F)c2)no1] | 1 | Ex. 1 (step 1 - 2), Ex. 18 (step 3) | DMSO-d₆: 13.42 (br, 1H), 9.36 (s, 1H), 8.55 (d, J = 2.4 Hz, 1H), 8.38 (d, J = 2.4 Hz, 1H), 8.09- 8.04 (m, 1H), 7.75-7.70 (m, 1H), 7.59-7.56 (m, 1H). | 342.1 [M + H]⁺ |
| 20 | 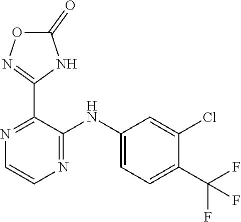 [SMILES: O=c1[nH]c(-c2nccnc2Nc2ccc(C(F)(F)F)c(Cl)c2)no1] | 1 | Ex. 1 (step 1 - 2), Ex. 18 (step 3) | DMSO-d₆: 13.42 (br, 1H), 9.31 (s, 1H), 8.55 (d, J = 2.4 Hz, 1H), 8.37 (d, J = 2.4 Hz, 1H), 8.20 (d, J = 1.6 Hz, 1H), 7.82-7.74 (m, 2H). | 358.1, 360.1 [M + H]⁺ |
| 21 | 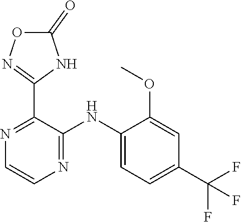 [SMILES: COc1cc(C(F)(F)F)ccc1Nc1nccnc1-c1noc(=O)[nH]1] | 1 | Ex. 1 (step 1 - 2), Ex. 18 (step 3) | DMSO-d₆: 13.38 (br, 1H), 9.73 (s, 1H), 8.76 (d, J = 8.8 Hz, 1H), 8.54 (d, J = 2.4 Hz, 1H), 8.31 (d, J = 2.4 Hz, 1H), 7.38-7.35 (m, 2H), 4.01 (s, 3H). | 354.1 [M + H]⁺ |
| 22 | 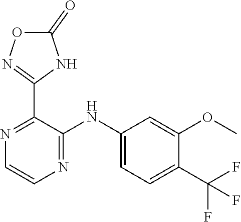 [SMILES: COc1cc(Nc2nccnc2-c2noc(=O)[nH]2)ccc1C(F)(F)F] | 1 | Ex. 1 (step 1 - 2), Ex. 18 (step 3) | DMSO-d₆: 13.40 (br, 1H), 9.19 (s, 1H), 8.52 (d, J = 2.4 Hz, 1H), 8.32 (d, J = 2.4 Hz, 1H), 7.60- 7.55 (m, 2H), 7.46 (d, J = 8.4 Hz, 1H), 3.90 (s, 3H). | 354.1 [M + H]⁺ |
| 23 | 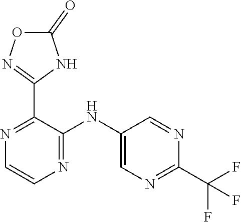 [SMILES: O=c1[nH]c(-c2nccnc2Nc2cnc(C(F)(F)F)nc2)no1] | 1 | Ex. 1 (step 1 - 2), Ex. 18 (step 3) | DMSO-d₆: 13.45 (br, 1H), 9.40 (s, 1H), 9.37 (s, 2H), 8.53 (d, J = 2.4 Hz, 1H), 8.42 (d, J = 2.4 Hz, 1H). | 326.1 [M + H]⁺ |
| 24 | 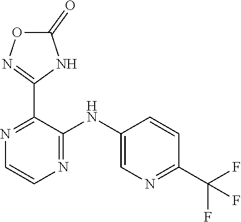 [SMILES: O=c1[nH]c(-c2nccnc2Nc2ccc(C(F)(F)F)nc2)no1] | 1 | Ex. 1 (step 1 - 2), Ex. 18 (step 3) | DMSO-d₆: 13.42 (br, 1H), 9.29 (s, 1H), 8.99 (d, J = 2.4 Hz, 1H), 8.52 (d, J = 2.4 Hz, 1H), 8.45 (dd, J = 8.8, 2.4 Hz, 1H), 8.37 (d, J = 2.4 Hz, 1H), 7.90 (d, J = 8.8 Hz, 1H). | 325.1 [M + H]⁺ |
| 25 | 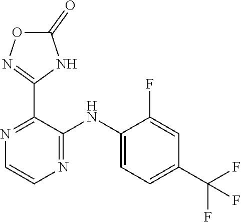 [SMILES: O=c1[nH]c(-c2nccnc2Nc2ccc(C(F)(F)F)cc2F)no1] | 1 | Ex. 1 (step 1 - 2), Ex. 18 (step 3) | DMSO-d₆: 13.48 (br, 1H), 9.41 (s, 1H), 8.69-8.64 (m, 1H), 8.55 (d, J = 2.4 Hz, 1H), 8.37 (d, J = 2.4 Hz, 1H), 7.83-7.80 (m, 1H), 7.65 (d, J = 8.4 Hz, 1H). | 342.1 [M + H]⁺ |
| 26 | 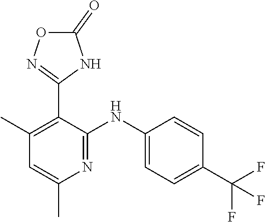 [SMILES: Cc1cc(C)c(-c2noc(=O)[nH]2)c(Nc2ccc(C(F)(F)F)cc2)n1] | 1 | Ex. 1 (step 1 - 2) | DMSO-d₆: 12.46 (br, 1H), 8.73 (s, 1H), 7.85 (d, J = 8.6 Hz, 2H), 7.60 (d, J = 8.6 Hz, 2H), 6.81 (s, 1H), 2.39 (s, 3H), 2.22 (s, 3H). | 351.2 [M + H]⁺ |
| 27 | 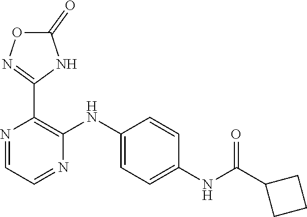 [SMILES: O=C(Nc1ccc(Nc2nccnc2-c2noc(=O)[nH]2)cc1)C1CCC1] | 2 | Ex. 14 (step 3) | DMSO-d₆: 13.32 (br, 1H), 9.72 (s, 1H), 8.84 (s, 1H), 8.40 (d, J = 2.4 Hz, 1H), 8.18 (d, J = 2.4 Hz, 1H), 7.61-7.54 (m, 4H), 3.26-3.18 (m, 1H), 2.25-2.19 (m, 2H), 2.11-2.06 (m, 2H), 1.97-1.89 (m, 1H), 1.85-1.75 (m, 1H). | 353.2 [M + H]⁺ |
| 28 | 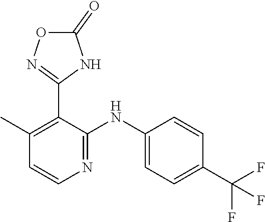 [SMILES: Cc1ccnc(Nc2ccc(C(F)(F)F)cc2)c1-c1noc(=O)[nH]1] | 1 | Ex. 1 (step 1 - 2), Ex. 26 (step 3) | DMSO-d₆: 12.52 (br, 1H), 8.79 (s, 1H), 8.24 (d, J = 5.2 Hz, 1H), 7.82 (d, J = 8.8 Hz, 2H), 7.61 (d, J = 8.8 Hz, 2H), 6.93 (d, J = 5.2 Hz, 1H), 2.27 (s, 3H). | 337.2 [M + H]⁺ |
| 29 | 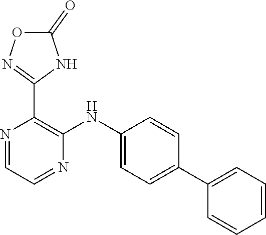 [SMILES: O=c1[nH]c(-c2nccnc2Nc2ccc(-c3ccccc3)cc2)no1] | 1 | Ex. 1 (step 1 - 2), Ex. 18 (step 3) | DMSO-d₆: 13.39 (br, 1H), 9.04 (s, 1H), 8.47 (d, J = 2.2 Hz, 1H), 8.24 (d, J = 2.2 Hz, 1H), 7.68 (d, J = 8.8 Hz, 2H), 7.72-7.65 (m, 4H), 7.48-7.43 (m, 2H), 7.37-7.31 (m, 1H). | 332.2 [M + H]⁺ |
| 30 | 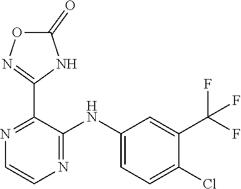 [SMILES: O=c1[nH]c(-c2nccnc2Nc2ccc(Cl)c(C(F)(F)F)c2)no1] | 1 | Ex. 1 (step 1 - 2), Ex. 18 (step 3) | DMSO-d₆: 13.38 (br, 1H), 9.16 (s, 1H), 8.48 (d, J = 2.4 Hz, 1H), 8.31 (d, J = 2.4 Hz, 1H), 8.25 (d, J = 2.4 Hz, 1H) 7.93 (dd, J = 8.8, 2.4 Hz, 1H), 7.69 (d, J = 8.8 Hz, 1H). | 358.1, 360.1 [M + H]⁺ |
| 31 | 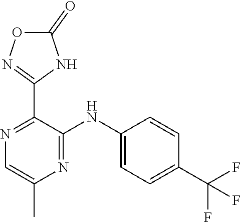 [SMILES: Cc1cnc(-c2noc(=O)[nH]2)c(Nc2ccc(C(F)(F)F)cc2)n1] | 1 | Ex. 1 (step 1 - 2) | DMSO-d₆: 13.32 (br, 1H), 9.19 (s, 1H), 8.23 (s, 1H), 7.94 (d, J = 8.6 Hz, 2H), 7.71 (d, J = 8.6 Hz, 2H), 2.49 (s, 3H). | 338.1 [M + H]⁺ |
| 32 | 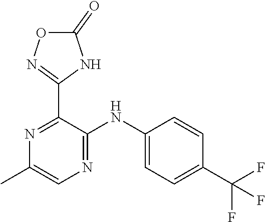 [SMILES: Cc1cnc(Nc2ccc(C(F)(F)F)cc2)c(-c2noc(=O)[nH]2)n1] | 1 | Ex. 31 (step 1 - 7) | DMSO-d₆: 13.22 (br, 1H), 9.08 (s, 1H), 8.40 (s, 1H), 7.86 (d, J = 8.8 Hz, 2H), 7.68 (d, J = 8.8 Hz, 2H), 2.50 (s, 3H). | 338.3 [M + H]⁺ |
| 33 | 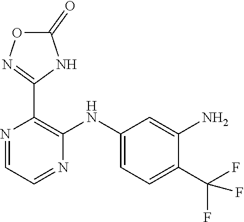 [SMILES: Nc1cc(Nc2nccnc2-c2noc(=O)[nH]2)ccc1C(F)(F)F] | 1 | Ex. 1 (step 1 - 2), Ex. 18 (step 3) | DMSO-d₆: 13.36 (br, 1H), 8.97 (s, 1H), 8.48 (d, J = 2.4 Hz, 1H), 8.26 (d, J = 2.4 Hz, 1H), 7.31 (d, J = 8.8 Hz, 1H), 7.17 (d, J = 1.4 Hz, 1H), 7.05 (dd, J = 8.8, 1.4 Hz, 1H), 5.59 (br, 1H). | 339.1 [M + H]⁺ |
| 34 | 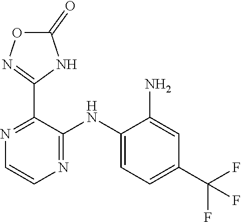 [SMILES: Nc1cc(C(F)(F)F)ccc1Nc1nccnc1-c1noc(=O)[nH]1] | 1 | Ex. 1 (step 1 - 2), Ex. 18 (step 3) and Ex. 33 (step 4) | DMSO-d₆: 13.31 (br, 1H), 8.38 (s, 1H), 8.33 (d, J = 2.4 Hz, 1H), 8.17 (d, J = 2.4 Hz, 1H), 7.59 (d, J = 8.8 Hz, 1H), 6.91 (d, J = 2.0 Hz, 1H), 8.90 (dd, J = 8.8, 2.0 Hz, 1H), 5.41 (br, 1H). | 339.0 [M + H]⁺ |
| 35 | 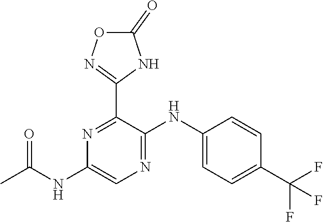 [SMILES: CC(=O)Nc1cnc(Nc2ccc(C(F)(F)F)cc2)c(-c2noc(=O)[nH]2)n1] | 1 | Ex. 1 (step 1 - 2), Ex. 2 (step 3) | DMSO-d₆: 13.18 (br, 1H), 10.52 (s, 1H), 9.18 (s, 1H), 9.01 (s, 1H), 7.82 (d, J = 8.6 Hz, 2H), 7.67 (d, J = 8.6 Hz, 2H), 2.14 (s, 3H). | 381.1 [M + H]⁺ |
| 36 | 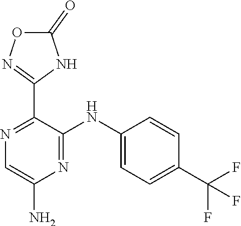 [SMILES: Nc1cnc(-c2noc(=O)[nH]2)c(Nc2ccc(C(F)(F)F)cc2)n1] | 1 | Ex. 1 (step 1 - 2), Ex. 4 (step 5) | DMSO-d₆: 12.79 (br, 1H), 8.97 (s, 1H), 7.93 (d, J = 8.8 Hz, 2H), 7.63 (d, J = 8.8 Hz, 2H), 7.54 (s, 1H), 7.34 (s, 2H). | 339.1 [M + H]⁺ |
| 37 | 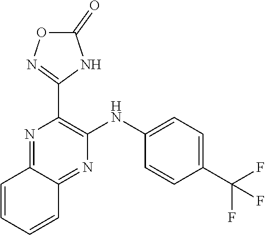 [SMILES: O=c1[nH]c(-c2nc3ccccc3nc2Nc2ccc(C(F)(F)F)cc2)no1] | 1 | Ex. 1 (step 1 - 2), Ex. 36 (step 5) | DMSO-d₆: 13.58 (br, 1H), 9.57 (s, 1H), 8.16 (d, J = 8.4 Hz, 2H), 8.03 (d, J = 8.0 Hz, 1H), 7.89- 7.86 (m, 2H), 7.78-7.75 (m, 2H), 7.71-7.66 (m, 1H). | 374.1 [M + H]⁺ |
| 38 | 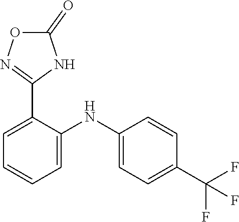 [SMILES: O=c1[nH]c(-c2ccccc2Nc2ccc(C(F)(F)F)cc2)no1] | 1 | Ex. 5 (step 1), Ex. 1 (step 2 - 3) | DMSO-d₆: 12.75 (s, 1H), 8.51 (s, 1H), 7.65 (dd, J = 7.8, 1.4 Hz, 1H), 7.59 (d, J = 8.5 Hz, 2H), 7.55-7.47 (m, 2H), 7.26-7.12 (m, 3H). | 319.6 [M − H]⁻ |
| 39 | 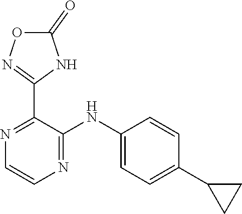 [SMILES: O=c1[nH]c(-c2nccnc2Nc2ccc(C3CC3)cc2)no1] | 2 | Ex. 14 (step 3) | DMSO-d₆: 13.32 (s, 1H), 8.87 (s, 1H), 8.39 (d, J = 2.0 Hz, 1H), 8.17 (d, J = 2 Hz, 1H), 7.51 (d, J = 8.2 Hz, 2H), 7.08 (d, J = 8.2 Hz, 2H), 1.94-1.86 (m, 1H), 0.95-0.90 (m, 2H), 0.66-0.62 (m, 2H). | 296.1 [M + H]⁺ |
| 40 | 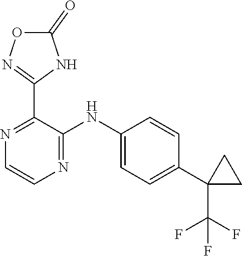 [SMILES: O=c1[nH]c(-c2nccnc2Nc2ccc(C3(C(F)(F)F)CC3)cc2)no1] | 1 | Ex. 1 (step 1 - 2), Ex. 18 (step 3) | DMSO-d₆: 13.36 (s, 1H), 8.98 (s, 1H), 8.44 (d, J = 2.4 Hz, 1H), 8.24 (d, J = 2.4 Hz, 1H), 7.67 (d, J = 8.6 Hz, 2H), 7.45 (d, J = 8.6 Hz, 2H), 1.34-1.31 (m, 2H), 1.12-1.09 (m, 2H). | 364.3 [M + H]⁺ |
| 41 | 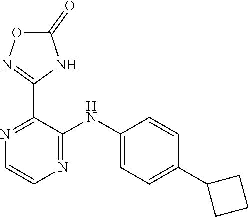 [SMILES: O=c1[nH]c(-c2nccnc2Nc2ccc(C3CCC3)cc2)no1] | 2 | Ex. 14 (step 3) | DMSO-d₆: 13.32 (s, 1H), 8.91 (s, 1H), 8.40 (d, J = 2.4 Hz, 1H), 8.18 (d, J = 2.4 Hz, 1H), 7.56 (d, J = 8.4 Hz, 2H), 7.23 (d, J = 8.4 Hz, 2H), 3.52-3.48 (m, 1H), 2.32-2.25 (m, 2H), 2.12- 2.01 (m, 2H), 2.00-1.91 (m, 1H), 1.84-1.78 (m, 1H). | 310.2 [M + H]⁺ |
| 42 | 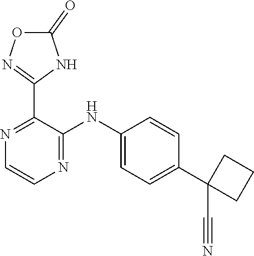 [SMILES: N#CC1(c2ccc(Nc3nccnc3-c3noc(=O)[nH]3)cc2)CCC1] | 1 | Ex. 1 (step 1 - 2), Ex. 18 (step 3) | DMSO-d₆: 13.36 (br, 1H), 9.11 (s, 1H), 8.44 (m, 1H), 8.23 (m, 1H), 7.72 (d, J = 8.4 Hz, 2H), 7.45 (d, J = 8.4 Hz, 2H), 2.76- 2.55 (m, 4H), 2.33-2.21 (m, 1H), 2.07-1.97 (m, 1H). | 335.3 [M + H]⁺ |
| 43 | 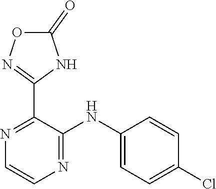 [SMILES: O=c1[nH]c(-c2nccnc2Nc2ccc(Cl)cc2)no1] | 1 | Ex. 1 (step 1 - 2), Ex. 18 (step 3) | DMSO-d₆: 13.36 (s, 1H), 8.99 (s, 1H), 8.44 (d, J = 2.4 Hz, 1H), 8.24 (d, J = 2.4 Hz, 1H), 7.71 (d, J = 8.8 Hz, 2H), 7.41 (d, J = 8.8 Hz, 2H). | 290.2, 292.2 [M + H]⁺ |
| 44 | 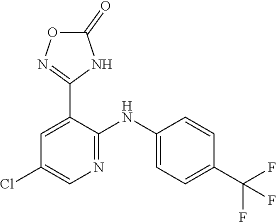 [SMILES: O=c1[nH]c(-c2cc(Cl)cnc2Nc2ccc(C(F)(F)F)cc2)no1] | 1 | Ex. 1 (step 1 - 3) | DMSO-d₆: 13.09 (br, 1H), 9.35 (s, 1H), 8.48 (d, J = 2.4 Hz, , 1H), 8.16 (d, J = 2.4 Hz, 1H), 7.85 (d, J = 8.6 Hz, 2H), 7.67 (d, J = 8.6 Hz, 2H). | 357.2, 359.2 [M + H]⁺ |
| 45 | 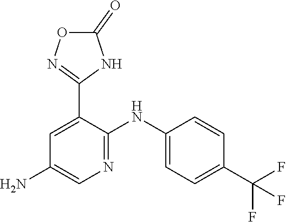 [SMILES: Nc1cnc(Nc2ccc(C(F)(F)F)cc2)c(-c2noc(=O)[nH]2)c1] | 1 | Ex. 1 (step 2 - 3) | DMSO-d₆ + D₂O: 7.91 (d, J = 2.8 Hz, 1H), 7.61 (d, J = 8.8 Hz, 2H), 7.53 (d, J = 8.8 Hz, 2H), 7.27 (d, J = 2.8 Hz, 1H). | 338.2 [M + H]⁺ |
| 46 | 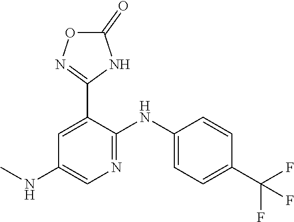 [SMILES: CNc1cnc(Nc2ccc(C(F)(F)F)cc2)c(-c2noc(=O)[nH]2)c1] | 1 | Ex. 36 (step 1), Ex. 1 (step 2 - 3), Ex. 5 (step 6) | DMSO-d₆: 12.97 (br, 1H), 8.93 (s, 1H), 7.91 (d, J = 2.8 Hz, 1H), 7.67 (d, J = 8.6 Hz, 2H), 7.54 (d, J = 8.6 Hz, 2H), 7.26 (d, J = 2.8 Hz, 1H), 5.83 (br, 1H), 2.72 (s, 3H). | 352.3 [M + H]⁺ |
| 47 | 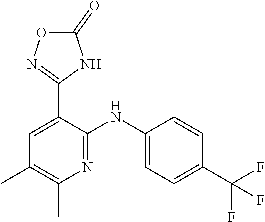 [SMILES: Cc1cc(-c2noc(=O)[nH]2)c(Nc2ccc(C(F)(F)F)cc2)nc1C] | 1 | Ex. 1 (step 1 - 2), Ex. 18 (step 3) | DMSO-d₆: 12.97 (br, 1H), 9.08 (s, 1H), 7.91 (d, J = 8.4 Hz, 2H), 7.81 (s, 1H), 7.63 (d, J = 8.4 Hz, 2H), 2.45 (s, 3H), 2.23 (s, 3H). | 351.2 [M + H]⁺ |
| 48 | 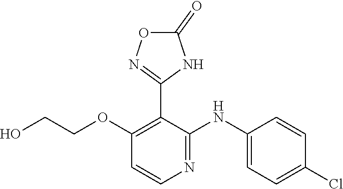 [SMILES: O=c1[nH]c(-c2c(OCCO)ccnc2Nc2ccc(Cl)cc2)no1] | 1 | Ex. 1 (step 1 - 2), Ex. 36 (step 1), Ex. 5 (step 6), Ex. 26 (step 3) | DMSO-d₆: 12.19 (s, 1H), 8.70 (s, 1H), 8.20 (d, J = 6.0 Hz, 1H), 7.62 (d, J = 8.8 Hz, 2H), 7.32 (d, J = 8.8 Hz, 2H), 6.77 (d, J = 6.0 Hz, 1H), 5.53 (br, 1H), 4.17 (t, J = 4.8 Hz, 2H), 3.71 (t, J = 4.8 Hz, 2H). | 349.2, 351.2 [M + H]⁺ |
| 49 | 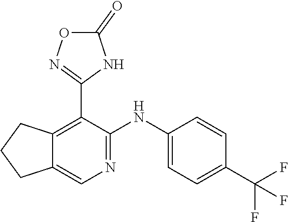 [SMILES: O=c1[nH]c(-c2c(Nc3ccc(C(F)(F)F)cc3)ncc3c2CCC3)no1] | 1 | Ex. 1 (step 1 - 2), Ex. 26 (step 3) | DMSO-d₆: 12.55 (s, 1H), 8.92 (s, 1H), 8.24 (s, 1H), 7.77 (d, J = 8.8 Hz, 2H), 7.59 (d, J = 8.8 Hz, 2H), 2.94-2.86 (m, 4H), 2.07-2.01 (m, 2H). | 363.2 [M + H]⁺ |
| 50 | 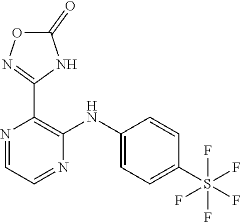 [SMILES: O=c1[nH]c(-c2nccnc2Nc2ccc(S(F)(F)(F)(F)F)cc2)no1] | 1 | Ex. 1 (step 1 - 2), Ex. 36 (step 5) | DMSO-d₆: 13.41 (s, 1H), 9.27 (s, 1H), 8.51 (d, J = 2.4 Hz, 1H), 8.34 (d, J = 2.4 Hz, 1H), 7.90- 7.86 (m, 4H). | 382.3 [M + H]⁺ |
| 51 | 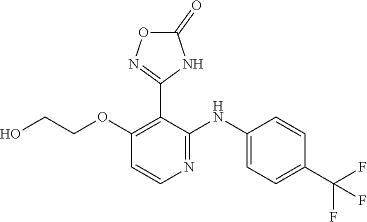 [SMILES: O=c1[nH]c(-c2c(OCCO)ccnc2Nc2ccc(C(F)(F)F)cc2)no1] | 1 | Ex. 48 (step 1 - 6) | DMSO-d₆: 12.25 (s, 1H), 8.92 (s, 1H), 8.27 (d, J = 6.0 Hz, 1H), 7.81 (d, J = 8.6 Hz, 2H), 7.61 (d, J = 8.6 Hz, 2H), 6.85 (d, J = 6.0 Hz, 1H), 5.25 (br, 1H), 4.18 (t, J = 4.8 Hz, 2H), 3.71 (t, J = 4.8 Hz, 2H). | 383.1 [M + H]⁺ |
| 52 | 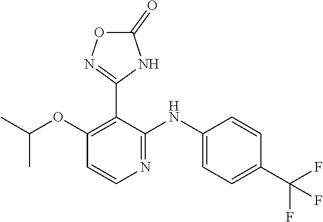 [SMILES: CC(C)Oc1ccnc(Nc2ccc(C(F)(F)F)cc2)c1-c1noc(=O)[nH]1] | 1 | Ex. 1 (step 2), Ex. 26 Step 3) | DMSO-d₆: 12.30 (s, 1H), 8.82 (s, 1H), 8.24 (d, J = 6.0 Hz, 1H), 7.81 (d, J = 8.8 Hz, 2H), 7.60 (d, J = 8.8 Hz, 2H), 6.82 (d, J = 6.0 Hz, 1H), 4.82-4.75 (m, 1H), 1.28 (d, J = 6.0 Hz, 6H). | 381.1 [M + H]⁺ |
| 53 | 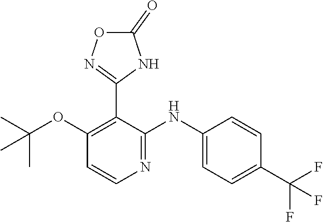 [SMILES: CC(C)(C)Oc1ccnc(Nc2ccc(C(F)(F)F)cc2)c1-c1noc(=O)[nH]1] | | Ex. 1 (step 2), Ex. 26 Step 3) | DMSO-d₆: 12.31 (s, 1H), 8.86 (br, 1H), 8.19 (d, J = 6.0 Hz, 1H), 7.81 (d, J = 8.8 Hz, 2H), 7.60 (d, J = 8.8 Hz, 2H), 6.88 (d, J = 6.0 Hz, 1H), 1.44 (s, 9H). | 395.2 [M + H]⁺ |
| 54 | 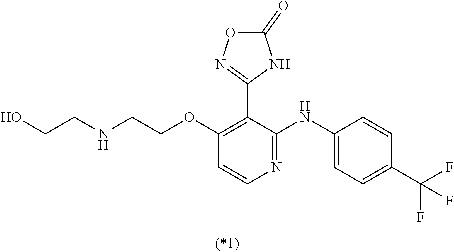 [SMILES: O=c1[nH]c(-c2c(OCCNCCO)ccnc2Nc2ccc(C(F)(F)F)cc2)no1] | 1 | Ex. 51 | DMSO-d₆ + D₂O: 8.32 (d, J = 6.0 Hz, 1H), 7.80 (d, J = 8.4 Hz, 2H), 7.62 (d, J = 8.4 Hz, 2H), 6.84 (d, J = 6.0 Hz, 1H), 4.43 (t, J = 4.4 Hz, 2H), 3.64 (t, J = 5.2 Hz, 2H), 3.41 (t, J = 4.4 Hz, 2H), 3.06 (t, J = 5.2 Hz, 2H). | 426.1 [M + H]⁺ |
| 55 | 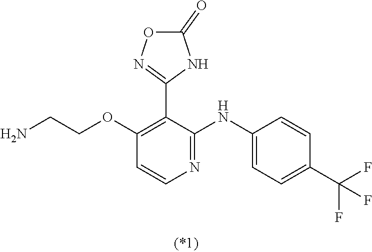 [SMILES: NCCOc1ccnc(Nc2ccc(C(F)(F)F)cc2)c1-c1noc(=O)[nH]1] | 1 | Ex. 54 (step 1) | DMSO-d₆ + D₂O: 8.31 (d, J = 6.0 Hz, 1H), 7.80 (d, J = 8.8 Hz, 2H), 7.62 (d, J = 8.8 Hz, 2H), 6.83 (d, J = 6.0 Hz, 1H), 4.32 (t, J = 4.8 Hz, 2H), 3.24 (t, J = 4.8 Hz, 2H). | 382.0 [M + H]⁺ |
| 56 | 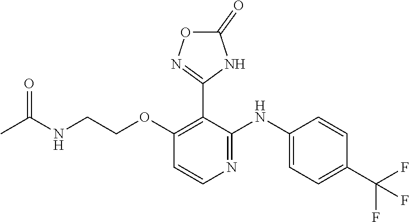 [SMILES: CC(=O)NCCOc1ccnc(Nc2ccc(C(F)(F)F)cc2)c1-c1noc(=O)[nH]1] | 1 | Ex. 55 | DMSO-d₆: 12.22 (s, 1H), 8.96 (s, 1H), 8.27 (d, J = 6.0 Hz, 1H), 8.03 (t, J = 5.6 Hz, 1H), 7.81 (d, J = 8.8 Hz, 2H), 7.61 (d, J = 8.8 Hz, 2H), 6.84 (d, J = 6.0 Hz, 1H), 4.14 (t, J = 5.6 Hz, 2H), 3.45-3.38 (m, 2H), 1.81 (s, 3H). | 424.1 [M + H]⁺ |
| 57 | 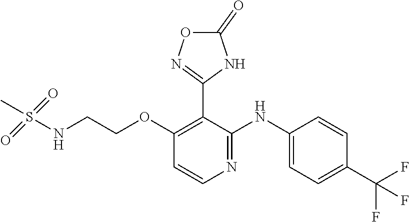 [SMILES: CS(=O)(=O)NCCOc1ccnc(Nc2ccc(C(F)(F)F)cc2)c1-c1noc(=O)[nH]1] | 1 | Ex. 55, Ex. 56 (step 1) | DMSO-d₆: 12.28 (s, 1H), 8.91 (s, 1H), 8.29 (d, J = 6.0 Hz, 1H), 7.81 (d, J = 8.6 Hz, 2H), 7.61 (d, J = 8.6 Hz, 2H), 7.29 (t, J = 5.6 Hz, 1H), 6.84 (d, J = 6.0 Hz, 1H), 4.19 (t, J = 5.6 Hz, 2H), 3.37-3.32 (m, 2H), 2.91 (s, 3H). | 460.2 [M + H]⁺ |
| 58 | 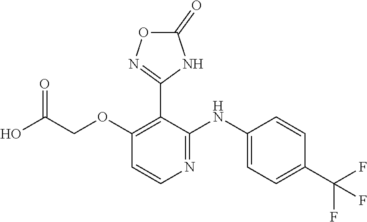 [SMILES: O=C(O)COc1ccnc(Nc2ccc(C(F)(F)F)cc2)c1-c1noc(=O)[nH]1] | 1 | Ex. 51 | DMSO-d₆: 13.36 (br, 1H), 12.64 (s, 1H), 9.02 (s, 1H), 8.26 (d, J = 6.0 Hz, 1H), 7.83 (d, J = 8.8 Hz, 2H), 7.62 (d, J = 8.8 Hz, 2H), 6.74 (d, J = 6.0 Hz, 1H), 4.90 (s, 2H). | 397.1 [M + H]⁺ |
| 59 | 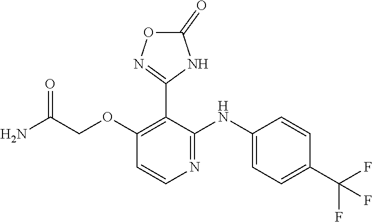 [SMILES: NC(=O)COc1ccnc(Nc2ccc(C(F)(F)F)cc2)c1-c1noc(=O)[nH]1] | 1 | Ex. 58 | DMSO-d₆: 12.48 (s, 1H), 9.09 (s, 1H), 8.29 (d, J = 6.0 Hz, 1H), 7.83 (d, J = 8.6 Hz, 2H), 7.63 (d, J = 8.6 Hz, 2H), 7.56 (s, 1H), 7.52 (s, 1H), 6.68 (d, J = 6.0 Hz, 1H), 4.70 (s, 2H). | 396.2 [M + H]⁺ |
| 60 | 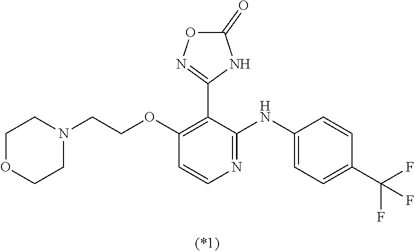 [SMILES: O=c1[nH]c(-c2c(OCCN3CCOCC3)ccnc2Nc2ccc(C(F)(F)F)cc2)no1] | 1 | Ex. 1 (step 1) | DMSO-d₆: 12.42 (br, 1H), 10.13 (br, 1H), 8.94 (s, 1H), 8.36 (d, J = 6.0 Hz, 1H), 7.83 (d, J = 8.6 Hz, 2H), 7.64 (d, J = 8.6 Hz, 2H), 6.90 (d, J = 6.0 Hz, 1H), 4.54 (t, J = 4.8 Hz, 2H), 4.04- 3.66 (m, 4H), 3.60 (t, J = 4.8 Hz, 2H), 3.46-3.18 (m, 4H). | 452.3 [M + H]⁺ |
| 61 | 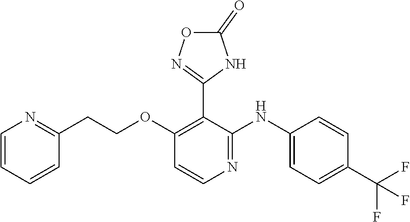 [SMILES: O=c1[nH]c(-c2c(OCCc3ccccn3)ccnc2Nc2ccc(C(F)(F)F)cc2)no1] | 1 | Ex. 60 (step 1 - 3) | DMSO-d₆: 12.98 (br, 1H), 9.18 (s, 1H), 8.58 (ddd, J = 5.0, 1.9, 0.9 Hz, 1H), 8.30 (d, J = 6.0 Hz, 1H), 7.85 (d, J = 8.6 Hz, 2H), 7.78 (td, J = 7.7, 1.9 Hz, 1H), 7.64 (d, J = 8.6 Hz, 2H), 7.42-7.26 (m, 2H), 6.88 (d, J = 6.0 Hz, 1H), 4.50 (t, J = 6.1 Hz, 2H), 3.24 (t, J = 6.1 Hz, 2H). | 444.2 [M + H]⁺ |
| 62 | 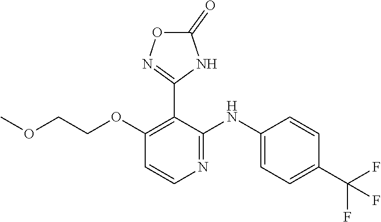 [SMILES: COCCOc1ccnc(Nc2ccc(C(F)(F)F)cc2)c1-c1noc(=O)[nH]1] | 1 | Ex. 60 (step 1 - 3) | DMSO-d₆: 12.34 (s, 1H), 8.90 (s, 1H), 8.30 (d, J = 6.0 Hz, 1H), 7.84 (d, J = 8.6 Hz, 2H), 7.63 (d, J = 8.6 Hz, 2H), 6.86 (d, J = 6.0 Hz, 1H), 4.32-4.26 (m, 2H), 3.71-3.65 (m, 2H), 3.30 (s, 3H). | 397.1 [M + H]⁺ |
| 63 | 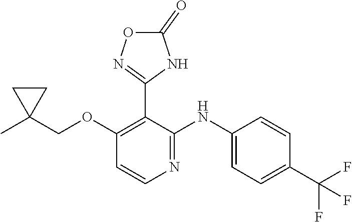 [SMILES: CC1(COc2ccnc(Nc3ccc(C(F)(F)F)cc3)c2-c2noc(=O)[nH]2)CC1] | 1 | Ex. 60 (step 1 - 3) | DMSO-d₆: 12.37 (s, 1H), 8.82 (s, 1H), 8.27 (d, J = 6.0 Hz, 1H), 7.83 (d, J = 8.6 Hz, 2H), 7.62 (d, J = 8.6 Hz, 2H), 6.78 (d, J = 6.0 Hz, 1H), 3.94 (s, 2H), 1.11 (s, 3H), 0.54-0.50 (m, 2H), 0.47-0.31 (m, 2H). | 407.1 [M + H]⁺ |
| 64 | 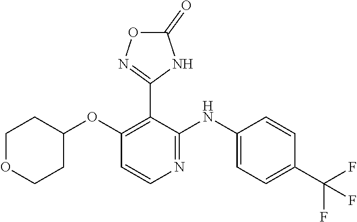 [SMILES: O=c1[nH]c(-c2c(OC3CCOCC3)ccnc2Nc2ccc(C(F)(F)F)cc2)no1] | 1 | Ex. 60 (step 1 - 3) | DMSO-d₆: 12.38 (s, 1H), 8.85 (s, 1H), 8.28 (d, J = 6.0 Hz, 1H), 7.85 (d, J = 8.6 Hz, 2H), 7.62 (d, J = 8.6 Hz, 2H), 6.92 (d, J = 6.0 Hz, 1H), 4.88-4.82 (m, 1H), 3.78-3.72 (m, 2H), 3.55-3.49 (m, 2H), 1.99-1.92 (m, 2H), 1.75-1.55 (m, 2H). | 423.1 [M + H]⁺ |
| 65 | 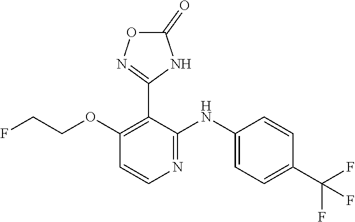 [SMILES: O=c1[nH]c(-c2c(OCCF)ccnc2Nc2ccc(C(F)(F)F)cc2)no1] | 1 | Ex. 60 (step 1 - 3) | DMSO-d₆: 12.41 (s, 1H), 8.91 (s, 1H), 8.31 (d, J = 5.9 Hz, 1H), 7.84 (d, J = 8.6 Hz, 2H), 7.63 (d, J = 8.6 Hz, 2H), 6.87 (d, J = 5.9 Hz, 1H), 4.84-4.78 (m, 1H), 4.71-4.66 (m, 1H), 4.50-4.46 (m, 1H), 4.42-4.39 (m, 1H). | 385.0 [M + H]⁺ |
| 66 | 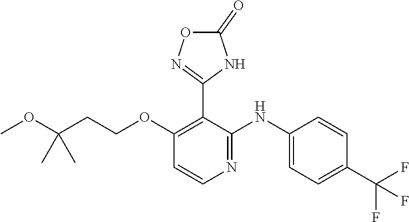 [SMILES: COC(C)(C)CCOc1ccnc(Nc2ccc(C(F)(F)F)cc2)c1-c1noc(=O)[nH]1] | 1 | Ex. 60 (step 1 - 3) | DMSO-d₆: 12.29 (s, 1H), 8.90 (s, 1H), 8.30 (d, J = 6.0 Hz, 1H), 7.84 (d, J = 8.6 Hz, 2H), 7.63 (d, J = 8.6 Hz, 2H), 6.86 (d, J = 6.0 Hz, 1H), 4.21 (t, J = 6.8 Hz, 2H), 3.12 (s, 3H), 1.92 (t, J = 6.8 Hz, 2H), 1.15 (s, 6H). | 439.3 [M + H]⁺ |
| 67 | 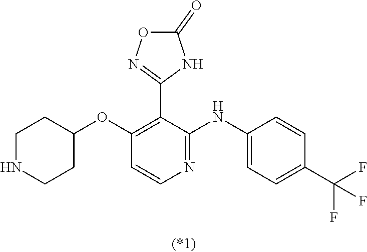 [SMILES: O=c1[nH]c(-c2c(OC3CCNCC3)ccnc2Nc2ccc(C(F)(F)F)cc2)no1] | 1 | Ex. 60 (step 1 - 2) | DMSO-d₆: 12.47 (s, 1H), 8.88 (s, 1H), 8.54 (br, 1H), 8.45 (br, 1H), 8.32 (d, J = 6.0 Hz, 1H), 7.83 (d, J = 8.6 Hz, 2H), 7.63 (d, J = 8.6 Hz, 2H), 6.92 (d, J = 6.0 Hz, 1H), 4.95-4.91 (m, 1H), 3.19-3.05 (m, 4H), 2.12-2.03 (m, 2H), 1.96- 1.84 (m, 2H). | 422.1 [M + H]⁺ |

### Preparation of Example 1: 3-[2-[4-(trifluoromethyl)anilino]-3-pyridyl]-4H-1,2,4-oxadiazol-5-one

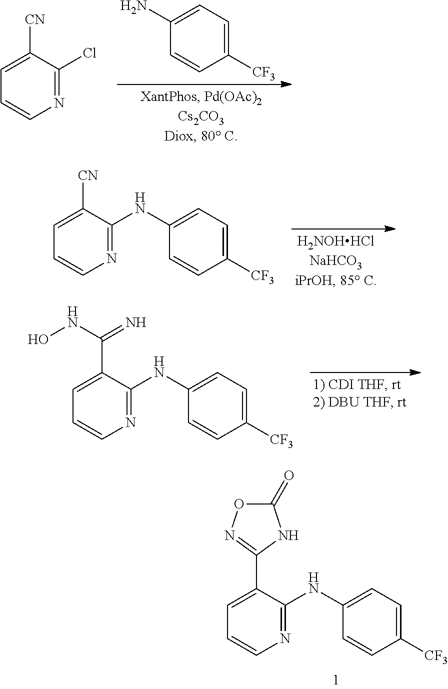

### Step 1: Preparation of 2-[4-(trifluoromethyl)anilino]pyridine-3-carbonitrile

To a stirred solution of 2-chloro-3-cyanopyridine (200 mg; 1.43 mmol) and 4-(trifluoromethyl)aniline (0.18 mL; 1.43 mmol) in dioxane (7 mL) were added Cs₂CO₃ (940 mg; 2.86 mmol), 4,5-bis(diphenylphospheno)-9,9-dimethylxanthene (87 mg; 0.143 mmol) and Pd(OAc)₂ (33 mg, 0.143 mmol). The reaction mixture was heated to 80° C. and was vigorously stirred for 1 h. After cooling, the mixture was filtered through a plug of Celite® and the cake was washed with EA. The combined filtrate was concentrated to dryness. The residue was purified by column chromatography (silica gel; c-Hex:EA; 1:0 to 1:1; v/v) to afford 2-[4-(trifluoromethyl)anilino]pyridine-3-carbonitrile (300 mg) as an off-white solid.

MS m/z (+ESI): 264.2 [M+H]⁺.

### Step 2: Preparation of N-hydroxy-2-[4-(trifluoromethyl)anilino]pyridine-3-carboxamidine

To a stirred suspension of hydroxylamine hydrochloride (86 mg; 1.23 mmol) in i-PrOH (4 mL) was added NaHCO₃ (153 mg; 1.80 mmol) and the mixture was stirred for 15 min. 2-[4-(trifluoromethyl)anilino]pyridine-3-carbonitrile (200 mg; 0.72 mmol) was added and the mixture heated to 85° C. After stirring for 1 h, the mixture was partitioned between EA and water. The organic layer was separated, washed with brine, dried over MgSO₄, filtered and concentrated to dryness to afford N-hydroxy-2-[4-(trifluoromethyl)anilino]pyridine-3-carboxamidine (216 mg) as off-white solid.

MS m/z (+ESI): 297.2 [M+H]⁺.

### Step 3: Preparation of 3-[2-[4-(trifluoromethyl)anilino]-3-pyridyl]-4H-1,2,4-oxadiazol-5-one

To a stirred solution of N-hydroxy-2-[4-(trifluoromethyl)anilino]pyridine-3-carboxamidine (50 mg; 0.16 mmol) in dry THF (2 mL) was added CDI (34 mg; 0.20 mmol). The reaction solution was stirred for 0.5 h and was then treated with DBU (0.035 mL; 0.24 mmol). After stirring for 1 h, the solution was concentrated to dryness. The residue was dissolved in EA and the solution was washed with citric acid solution, 10% in water and brine, dried over MgSO₄, filtered and concentrated. The residue was triturated in chloroform and the suspension was filtered and washed with chloroform. The solid was dried under high vacuum to afford 3-[2-[4-(trifluoromethyl)anilino]-3-pyridyl]-4H-1,2,4-oxadiazol-5-one (40 mg) as a white powder.

### Preparation of Example 2: 3-[3-[4-(trifluoromethyl)anilino]pyrazin-2-yl]-4H-1,2,4-oxadiazol-5-one

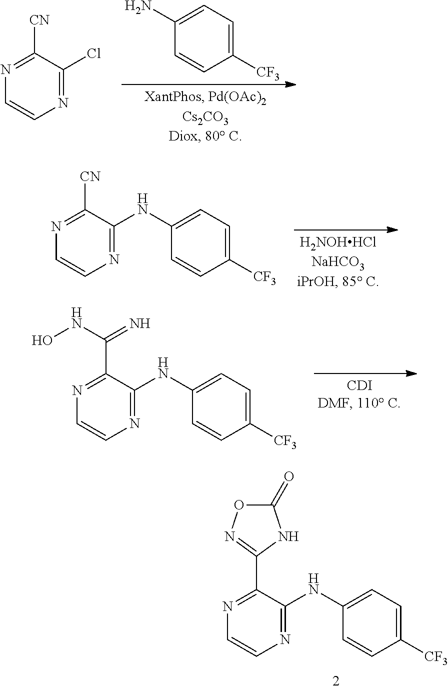

### Step 1: Preparation of 3-[4-(trifluoromethyl)anilino]pyrazine-2-carbonitrile

The title compound was prepared as a yellow solid following scheme 1 and in analogy to Example 1 (step 1) using 3-chloropyrazine-2-carbonitrile and 4-(trifluoromethyl)aniline as starting materials, and after purification by column chromatography (silica gel; PE:EA; 6:1; v/v).

MS m/z (+ESI): 264.9 [M+H]⁺.

¹H-NMR (400 MHz, DMSO-d₆) δ ppm: 9.89 (s, 1H), 8.50 (d, J=2.4 Hz, 1H), 8.27 (d, J=2.4 Hz, 1H), 7.78 (d, J=8.8 Hz, 2H), 7.68 (d, J=8.8 Hz, 2H).

### Step 2: Preparation of N-hydroxy-3-[4-(trifluoromethyl)anilino]pyrazine-2-carboxamidine

The title compound was prepared as a yellow solid following scheme 1 and in analogy to Example 1 (step 2) using 3-[4-(trifluoromethyl)anilino]pyrazine-2-carbonitrile as starting material.

MS m/z (+ESI): 298.5 [M+H]⁺.

¹H-NMR (400 MHz, DMSO-d₆) δ ppm: 11.62 (s, 1H), 10.59 (s, 1H), 8.29 (d, J=2.4 Hz, 1H), 8.13 (d, J=2.4 Hz, 1H), 7.90 (d, J=8.6 Hz, 2H), 7.71 (d, J=8.6 Hz, 2H), 6.26 (s, 2H).

### Step 3: Preparation of 3-[3-[4-(trifluoromethyl)anilino]pyrazin-2-yl]-4H-1,2,4-oxadiazol-5-one

To a solution of CDI (601 mg; 3.63 mmol) in DMF (8 mL) was added N-hydroxy-3-[4-(trifluoromethyl)anilino]pyrazine-2-carboxamidine (400 mg; 1.21 mmol). The solution was heated to 110° C. and stirred for 1 h. The solution was then subjected to purification by preparative HPLC to afford 3-[3-[4-(trifluoromethyl)anilino]pyrazin-2-yl]-4H-1,2,4-oxadiazol-5-one (85 mg) as a light yellow solid.

### Preparation of Example 4: 3-[6-amino-3-[4-(trifluoromethyl)anilino]pyrazin-2-yl]-4H-1,2,4-oxadiazol-5-one

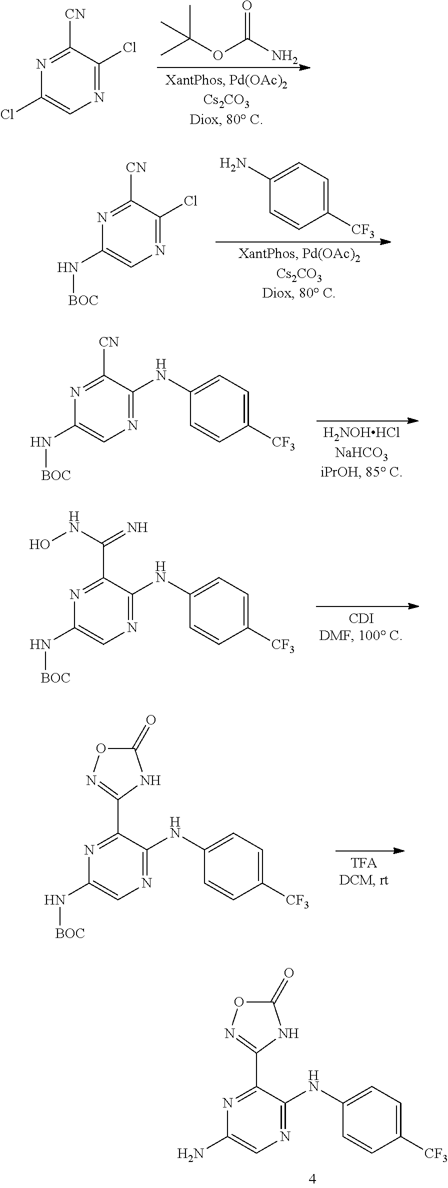

### Step 1: Preparation of tert-butyl N-(5-chloro-6-cyano-pyrazin-2-yl)carbamate

The title compound was prepared as a white solid following scheme 1 and in analogy to Example 1 (step 1) using 3,6-dichloropyrazine-2-carbonitrile and tert-butyl carbamate as starting materials and after purification by column chromatography (silica gel; PE:EA; 1:0 to 4:1; v/v).

MS m/z (+ESI): 253.1, 255.1 [M+H]⁺.

¹H-NMR (400 MHz, CDCl₃) δ ppm: 10.88 (s, 1H), 9.10 (s, 1H), 1.49 (s, 9H).

### Step 2: Preparation of tert-butyl N-[6-cyano-5-[4-(trifluoromethyl)anilino]pyrazin-2-yl]carbamate

The title compound was prepared as a white solid following scheme 1 and in analogy to Example 1 (step 1) using tert-butyl N-(5-chloro-6-cyano-pyrazin-2-yl)carbamate and 4-(trifluoromethyl)aniline as starting materials and after purification by column chromatography (silica gel; PE:EA; 1:0 to 3:1; v/v).

MS m/z (+ESI): 380.1 [M+H]⁺.

¹H-NMR (400 MHz, CDCl₃) δ ppm: 9.16 (s, 1H), 7.69 (d, J=8.8 Hz, 2H), 7.62 (d, J=8.8 Hz, 2H), 7.05-7.03 (m, 2H).

### Step 3: Preparation of tert-butyl N-[6-(N-hydroxycarbamimidoyl)-5-[4-(trifluoromethyl)anilino]pyrazin-2-yl]carbamate

The title compound was prepared as a yellow solid following scheme 1 and in analogy to Example 1 (step 2) using tert-butyl N-[6-cyano-5-[4-(trifluoromethyl)anilino]pyrazin-2-yl]carbamate as starting material.

MS m/z (+ESI): 413.1 [M+H]⁺.

### Step 4: Preparation of tert-butyl N-[6-(5-oxo-4H-1,2,4-oxadiazol-3-yl)-5-[4-(trifluoromethyl)anilino]pyrazin-2-yl]carbamate

The title compound was prepared as a light yellow solid following scheme 1 and in analogy to Example 2 (step 3) using tert-butyl N-[6-(N-hydroxycarbamimidoyl)-5-[4-(trifluoromethyl)anilino]pyrazin-2-yl]carbamate as starting material and after purification by column chromatography (silica gel; DCM:MeOH; 1:0 to 9:1; v/v).

MS m/z (+ESI): 439.1 [M+H]⁺.

### Step 5: Preparation of 3-[6-amino-3-[4-(trifluoromethyl)anilino]pyrazin-2-yl]-4H-1,2,4-oxadiazol-5-one

To a solution of tert-butyl N-[6-(5-oxo-4H-1,2,4-oxadiazol-3-yl)-5-[4-(trifluoromethyl)anilino]pyrazin-2-yl]carbamate (30 mg; 0.065 mmol) in DCM (5 mL) was added TFA (0.24 mL; 3.25 mmol). The solution was stirred for 2 h and then concentrated to dryness. The residue was purified by preparative HPLC to afford 3-[6-amino-3-[4-(trifluoromethyl)anilino]pyrazin-2-yl]-4H-1,2,4-oxadiazol-5-one (15 mg) as a yellow solid.

### Preparation of Example 5: 3-[6-methoxy-3-[4-(trifluoromethyl)anilino]pyrazin-2-yl]-4H-1,2,4-oxadiazol-5-one

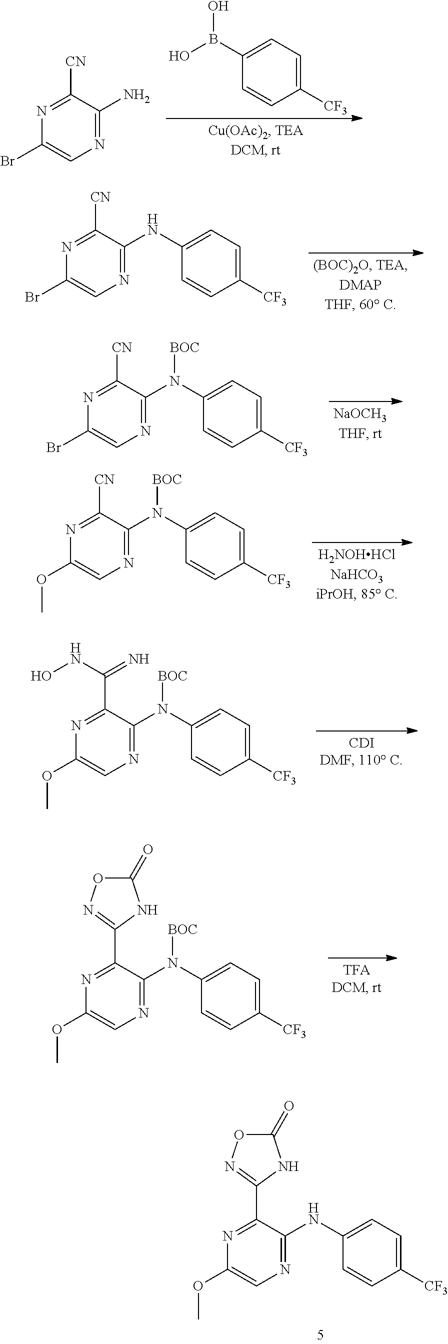

### Step 1: Preparation of 6-bromo-3-[4-(trifluoromethyl)anilino]pyrazine-2-carbonitrile

Under ambient air, to a solution of 3-amino-6-bromopyrazine-2-carbonitrile (1 000 mg; 4.87 mmol) and 4-(trifluoromethyl)benzeneboronic acid (1 889 mg; 9.75 mmol) in DCM (20 mL) was added copper(II) acetate monohydrate (1 986 mg; 9.75 mmol) and TEA (2.05 mL; 14.62 mmol). The suspension was stirred for 12 h. Solvent was removed under reduced pressure and the residue was purified by column chromatography (silica gel; PE:EA; 4:1; v/v) to afford 6-bromo-3-[4-(trifluoromethyl)anilino]pyrazine-2-carbonitrile (700 mg) as a yellow solid.

MS m/z (+ESI): 343.1, 345.1 [M+H]⁺.

¹H-NMR (400 MHz, DMSO-d₆) δ ppm: 10.07 (s, 1H), 8.67 (s, 1H), 7.74 (d, J=8.8 Hz, 2H), 7.69 (d, J=8.8 Hz, 2H).

### Step 2: Preparation of tert-butyl N-(5-bromo-3-cyano-pyrazin-2-yl)-N-[4-(trifluoromethyl)phenyl]carbamate

To a solution of 6-bromo-3-[4-(trifluoromethyl)anilino]pyrazine-2-carbonitrile (200 mg; 0.52 mmol) in THF (10 mL) was added DMAP (13 mg; 0.10 mmol), TEA (0.15 mL; 1.05 mmol) and (BOC)₂O (180 mg; 0.79 mmol). The solution was heated to 60° C. and stirred for 2 h. Volatiles were removed under reduced pressure and the residue was purified by column chromatography (silica gel; PE:EA; 1:0 to 4:1; v/v) to afford tert-butyl N-(5-bromo-3-cyano-pyrazin-2-yl)-N-[4-(trifluoromethyl)phenyl]carbamate (200 mg) as a light yellow solid.

MS m/z (+ESI): 443.0, 445.0 [M+H]⁺.

¹H-NMR (400 MHz, CDCl₃) δ ppm: 8.66 (s, 1H), 7.67 (d, J=8.4 Hz, 2H), 7.43 (d, J=8.4 Hz, 2H), 1.54 (s, 9H).

### Step 3: Preparation of tert-butyl N-(3-cyano-5-methoxy-pyrazin-2-yl)-N-[4-(trifluoromethyl)phenyl]carbamate

To a solution of tert-butyl N-(5-bromo-3-cyano-pyrazin-2-yl)-N-[4-(trifluoromethyl)phenyl]carbamate (100 mg; 0.21 mmol) in THF (3 mL) was added sodium methoxide (43 mg; 0.24 mmol). The suspension was stirred for 2 h. The reaction was deactivated by cautious addition of saturated aqueous solution of NH₄Cl (5 mL). The product was extracted with EA (10 mL). The organic layer was separated, dried over Na₂SO₄, filtered and concentrated. The residue was purified by column chromatography (silica gel; PE:EA; 1:0 to 4:1; v/v) to afford tert-butyl N-(3-cyano-5-methoxy-pyrazin-2-yl)-N-[4-(trifluoromethyl)phenyl]carbamate (50 mg) as a light yellow oil.

MS m/z (+ESI): 395.2 [M+H]⁺.

¹H-NMR (400 MHz, CDCl₃) δ ppm: 8.27 (s, 1H), 7.64 (d, J=8.8 Hz, 2H), 7.48 (d, J=8.8 Hz, 2H), 4.06 (s, 3H), 1.52 (s, 9H).

### Step 4: Preparation of tert-butyl N-[3-(N-hydroxycarbamimidoyl)-5-methoxy-pyrazin-2-yl]-N-[4-(trifluoromethyl)phenyl]carbamate

The title compound was prepared as a yellow solid following scheme 1 and in analogy to Example 1 (step 2) using tert-butyl N-(3-cyano-5-methoxy-pyrazin-2-yl)-N-[4-(trifluoromethyl)phenyl]carbamate as starting material.

MS m/z (+ESI): 428.1 [M+H]⁺.

### Step 5: Preparation of tert-butyl N-[5-methoxy-3-(5-oxo-4H-1,2,4-oxadiazol-3-yl)pyrazin-2-yl]-N-[4-(trifluoromethyl)phenyl]carbamate

The title compound was prepared as a light yellow solid following scheme 1 and in analogy to Example 2 (step 3) using tert-butyl N-[3-(N-hydroxycarbamimidoyl)-5-methoxy-pyrazin-2-yl]-N-[4-(trifluoromethyl)phenyl]carbamate as starting material and after purification by column chromatography (silica gel; DCM:MeOH; 1:0 to 9:1; v/v).

MS m/z (−ESI): 452.2 [M−H]⁻.

### Step 6: Preparation of 3-[6-methoxy-3-[4-(trifluoromethyl)anilino]pyrazin-2-yl]-4H-1,2,4-oxadiazol-5-one

To a solution of tert-butyl N-[5-methoxy-3-(5-oxo-4H-1,2,4-oxadiazol-3-yl)pyrazin-2-yl]-N-[4-(trifluoromethyl)phenyl]carbamate (100 mg; 0.22 mmol) in DCM (5 mL) was added TFA (0.83 mL; 11.03 mmol). The solution was stirred for 18 h and then concentrated to dryness. The residue was purified by preparative HPLC to afford 3-[6-methoxy-3-[4-(trifluoromethyl)anilino]pyrazin-2-yl]-4H-1,2,4-oxadiazol-5-one (50 mg) as a light yellow solid.

### Preparation of Example 6: 3-[6-(hydroxymethyl)-3-[4-(trifluoromethyl)anilino]pyrazin-2-yl]-4H-1,2,4-oxadiazol-5-one

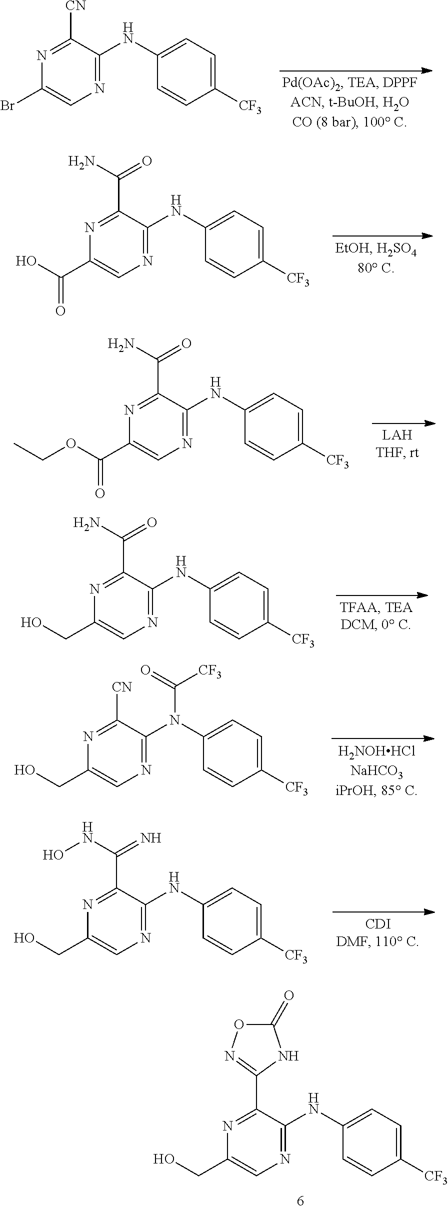

### Step 1: Preparation of 6-carbamoyl-5-[4-(trifluoromethyl)anilino]pyrazine-2-carboxylic acid

To a solution of 6-bromo-3-[4-(trifluoromethyl)anilino]pyrazine-2-carbonitrile (500 mg; 1.31 mmol) in ACN (15 mL), t-BuOH (10 mL) and H₂O (0.2 mL) was added Pd(OAc)₂ (44 mg; 0.2 mmol), TEA (0.55 mL; 3.93 mmol) and DPPF (134 mg; 0.24 mmol) in high-pressure autoclave. The suspension was charged with CO to 8 Bar and was then heated to 100° C. The suspension was stirred at this temperature for 12 h. Volatiles were removed under reduced pressure. The residue was purified by preparative HPLC to afford 6-carbamoyl-5-[4-(trifluoromethyl)anilino]pyrazine-2-carboxylic acid (150 mg) as a brown solid.

MS m/z (+ESI): 327.1 [M+H]⁺.

### Step 2: Preparation of ethyl 6-carbamoyl-5-[4-(trifluoromethyl)anilino]pyrazine-2-carboxylate

To a solution of 6-carbamoyl-5-[4-(trifluoromethyl)anilino]pyrazine-2-carboxylic acid (100 mg; 0.28 mmol) in EtOH (5 mL) was added 96% H₂SO₄ (0.1 mL). The solution was heated to 80° C. and was stirred for 12 h. The solution was diluted with EA and then washed with NaHCO₃ solution, 8% in water. The organic layer was separated, dried over Na₂SO₄, filtered and concentrated to afford ethyl 6-carbamoyl-5-[4-(trifluoromethyl)anilino]pyrazine-2-carboxylate (100 mg) as a yellow solid.

MS m/z (+ESI): 354.9 [M+H]⁺.

¹H-NMR (400 MHz, DMSO-d₆) δ ppm: 12.00 (s, 1H), 8.98 (s, 1H), 8.28 (s, 1H), 8.22 (s, 1H), 7.96 (d, J=8.8 Hz, 2H), 7.73 (d, J=8.8 Hz, 2H), 4.36 (q, J=7.2 Hz, 2H), 1.35 (t, J=7.2 Hz, 3H).

### Step 3: Preparation of 6-(hydroxymethyl)-3-[4-(trifluoromethyl)anilino]pyrazine-2-carboxamide

To a solution of ethyl 6-carbamoyl-5-[4-(trifluoromethyl)anilino]pyrazine-2-carboxylate (100 mg; 0.25 mmol) in THF (10 mL) was added LAH (29 mg; 0.76 mmol). The suspension was stirred for 1 h. H₂O was cautiously added and the product was extracted with EA. The organic layer was separated, washed with brine, dried over Na₂SO₄, filtered and concentrated. The residue was purified by column chromatography (silica gel; PE:EA; 1:1; v/v) to afford 6-(hydroxymethyl)-3-[4-(trifluoromethyl)anilino]pyrazine-2-carboxamide (45 mg) as a yellow solid.

MS m/z (+ESI): 313.1 [M+H]⁺.

¹H-NMR (400 MHz, DMSO-d₆) δ ppm: 11.59 (s, 1H), 8.52 (s, 1H), 8.49 (s, 1H), 8.07 (s, 1H), 7.90 (d, J=8.8 Hz, 2H), 7.67 (d, J=8.8 Hz, 2H), 5.41 (t, J=6.0 Hz, 1H), 4.58 (d, J=6.0 Hz, 2H).

### Step 4: Preparation of N-[3-cyano-5-(hydroxymethyl)pyrazin-2-yl]-2,2,2-trifluoro-N-[4-(trifluoromethyl)phenyl]acetamide

To a solution of 6-(hydroxymethyl)-3-[4-(trifluoromethyl)anilino]pyrazine-2-carboxamide (40 mg; 0.12 mmol) in DCM (5 mL) was added TFAA (0.05 mL; 0.35 mmol) and TEA (0.05 mL; 0.35 mmol) at 0° C. The solution was stirred for 2 h and volatiles were removed under reduced pressure. The residue was purified by column chromatography (silica gel; PE:EA; 5:1; v/v) to afford N-[3-cyano-5-(hydroxymethyl)pyrazin-2-yl]-2,2,2-trifluoro-N-[4-(trifluoromethyl)phenyl]acetamide (30 mg) as a yellow semisolid.

MS m/z (+ESI): 391.1 [M+H]⁺.

### Step 5: Preparation of N-hydroxy-6-(hydroxymethyl)-3-[4-(trifluoromethyl)anilino]pyrazine-2-carboxamidine

The title compound was prepared as a yellow solid following scheme 1 and in analogy to Example 1 (step 2) using N-[3-cyano-5-(hydroxymethyl)pyrazin-2-yl]-2,2,2-trifluoro-N-[4-(trifluoromethyl)phenyl]acetamide as starting material.

MS m/z (+ESI): 328.2 [M+H]⁺.

### Step 6: Preparation of 3-[6-(hydroxymethyl)-3-[4-(trifluoromethyl)anilino]pyrazin-2-yl]-4H-1,2,4-oxadiazol-5-one

The title compound was prepared as a light yellow solid following scheme 1 and in analogy to Example 2 (step 3) using N-hydroxy-6-(hydroxymethyl)-3-[4-(trifluoromethyl)anilino]pyrazine-2-carboxamidine as starting material and after purification by preparative HPLC.

### Preparation of Example 7: 3-[6-(2-aminoethyl)-3-[4-(trifluoromethyl)anilino]pyrazin-2-yl]-4H-1,2,4-oxadiazol-5-one, trifluoroacetic acid

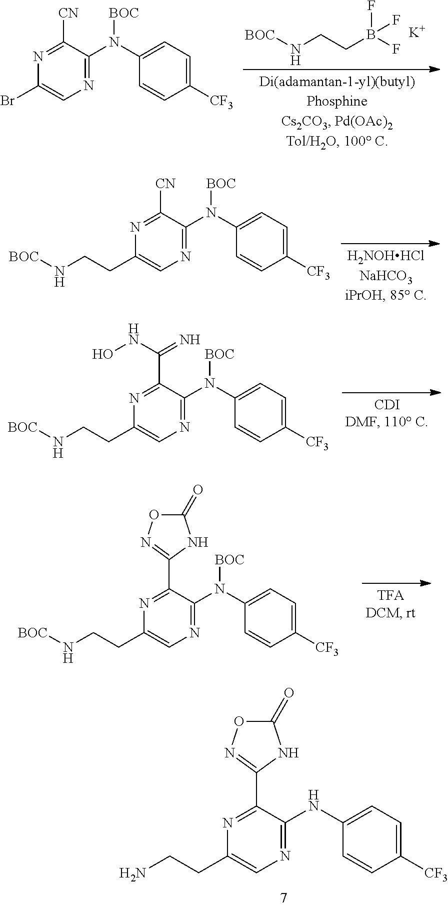

### Step 1: Preparation of tert-butyl N-[5-[2-(tert-butoxycarbonylamino)ethyl]-3-cyano-pyrazin-2-yl]-N-[4-(trifluoromethyl)phenyl]carbamate

Under argon atmosphere, to a solution of tert-butyl N-(5-bromo-3-cyano-pyrazin-2-yl)-N-[4-(trifluoromethyl)phenyl]carbamate (50 mg; 0.11 mmol) (intermediate from Example 5 step 2) and potassium (2-((tert-butoxycarbonyl)amino)ethyl)trifluoroborate (51 mg; 0.19 mmol) in Tol (4 mL) and H₂O (0.4 mL) was added Pd(OAc)₂ (10 mg; 0.02 mmol), di(adamantan-1-yl)(butyl)phosphine (8 mg; 0.02 mmol) and Cs₂CO₃ (88 mg; 0.27 mmol). The suspension was heated to 100° C. and stirred for 2 h. Volatiles were removed under reduced pressure. The residue was purified by column chromatography (silica gel; PE:EA; 2:1; v/v) to afford tert-butyl N-[5-[2-(tert-butoxycarbonylamino)ethyl]-3-cyano-pyrazin-2-yl]-N-[4-(trifluoromethyl)phenyl]carbamate (25 mg) as a yellow solid.

MS m/z (−ESI): 506.4 [M−H]⁻.

¹H-NMR (400 MHz, DMSO-d₆) δ ppm: 8.72 (s, 1H), 7.76 (d, J=8.4 Hz, 2H), 7.51 (d, J=8.4 Hz, 2H), 6.94 (t, J=5.6 Hz, 1H), 3.34-3.29 (m, 2H, overlap H₂O), 2.98 (t, J=6.4 Hz, 2H), 1.44 (s, 9H), 1.28 (s, 9H).

### Step 2: Preparation of tert-butyl N-[5-[2-(tert-butoxycarbonylamino)ethyl]-3-(N-hydroxycarbamimidoyl)pyrazin-2-yl]-N-[4-(trifluoromethyl)phenyl]carbamate

The title compound was prepared as a yellow solid following scheme 1 and in analogy to Example 1 (step 2) using tert-butyl N-[5-[2-(tert-butoxycarbonylamino)ethyl]-3-cyano-pyrazin-2-yl]-N-[4-(trifluoromethyl)phenyl]carbamate as starting material and after purification by column chromatography (silica gel; PE:EA; 2:1; v/v).

MS m/z (+ESI): 541.5 [M+H]⁺.

### Step 3: Preparation of tert-butyl N-[5-[2-(tert-butoxycarbonylamino)ethyl]-3-(5-oxo-4H-1,2,4-oxadiazol-3-yl)pyrazin-2-yl]-N-[4-(trifluoromethyl)phenyl]carbamate

The title compound was prepared as a yellow solid following scheme 1 and in analogy to Example 2 (step 3) using tert-butyl N-[5-[2-(tert-butoxycarbonylamino)ethyl]-3-(N-hydroxycarbamimidoyl)pyrazin-2-yl]-N-[4-(trifluoromethyl)phenyl]carbamate as starting material and after purification by preparative HPLC.

MS m/z (−ESI): 565.4 [M−H]⁻.

### Step 4: Preparation of 3-[6-(2-aminoethyl)-3-[4-(trifluoromethyl)anilino]pyrazin-2-yl]-4H-1,2,4-oxadiazol-5-one, trifluoroacetic acid

The title compound was prepared as a yellow solid following scheme 1 and in analogy to Example 5 (step 6) using tert-butyl N-[5-[2-(tert-butoxycarbonylamino)ethyl]-3-(5-oxo-4H-1,2,4-oxadiazol-3-yl)pyrazin-2-yl]-N-[4-(trifluoromethyl)phenyl]carbamate as starting material and after purification by preparative HPLC.

### Preparation of Example 9: 3-[3-[4-(trifluoromethyl)anilino]-2-pyridyl]-4H-1,2,4-oxadiazol-5-one

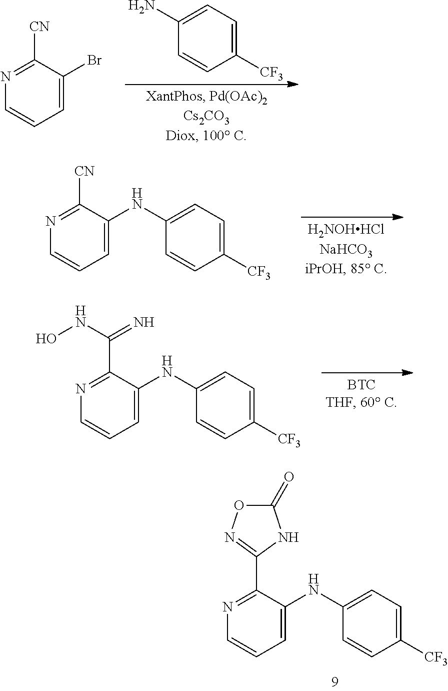

### Step 1: Preparation of 3-[4-(trifluoromethyl)anilino]pyridine-2-carbonitrile

The title compound was prepared as a white solid following scheme 1 and in analogy to Example 1 (step 1) using 3-bromopicolinonitrile and 4-(trifluoromethyl)aniline as starting materials at a temperature of 100° C. and after purification by column chromatography (silica gel; PE:EA; 8:1; v/v).

MS m/z (+ESI): 264.0 [M+H]⁺.

¹H-NMR (400 MHz, DMSO-d₆) δ ppm: 9.18 (s, 1H), 8.35 (dd; J=9.2, 1.2 Hz, 1H), 7.90 (dd, J=8.8, 1.2 Hz, 1H), 7.63-7.59 (m, 3H), 7.24 (d, J=8.8 Hz, 2H).

### Step 2: Preparation of N-hydroxy-3-[4-(trifluoromethyl)anilino]pyridine-2-carboxamidine

The title compound was prepared as a yellow solid following scheme 1 and in analogy to Example 1 (step 2) using 3-[4-(trifluoromethyl)anilino]pyridine-2-carbonitrile as starting material.

MS m/z (+ESI): 297.3 [M+H]⁺.

¹H-NMR (400 MHz, DMSO-d₆) δ ppm: 10.48 (s, 1H), 10.20 (s, 1H), 8.14 (dd, J=8.4, 1.4 Hz, 1H), 7.84 (dd, J=8.4, 1.6 Hz, 1H), 7.65 (d, J=8.8 Hz, 2H), 7.35-7.30 (m, 3H), 6.17 (br, 2H).

### Step 3: Preparation of 3-[3-[4-(trifluoromethyl)anilino]-2-pyridyl]-4H-1,2,4-oxadiazol-5-one

A solution of N-hydroxy-3-[4-(trifluoromethyl)anilino]pyridine-2-carboxamidine (200 mg; 0.61 mmol) in THF (20 mL) cooled to 0° C. was treated with BTC (184 mg; 0.61 mmol). The mixture was heated to 60° C. and stirred for 3 h. The solution was diluted with EA (60 mL) and water (60 mL). The organic layer was separated and successively washed with NaOH solution, 10% in water and brine The solution was dried over MgSO₄, filtered and concentrated to dryness. The residue was purified by preparative HPLC to afford 3-[3-[4-(trifluoromethyl)anilino]-2-pyridyl]-4H-1,2,4-oxadiazol-5-one (89 mg) as a white solid.

### Preparation of Example 12: 3-[5-methoxy-3-[4-(trifluoromethyl)anilino]pyrazin-2-yl]-4H-1,2,4-oxadiazol-5-one

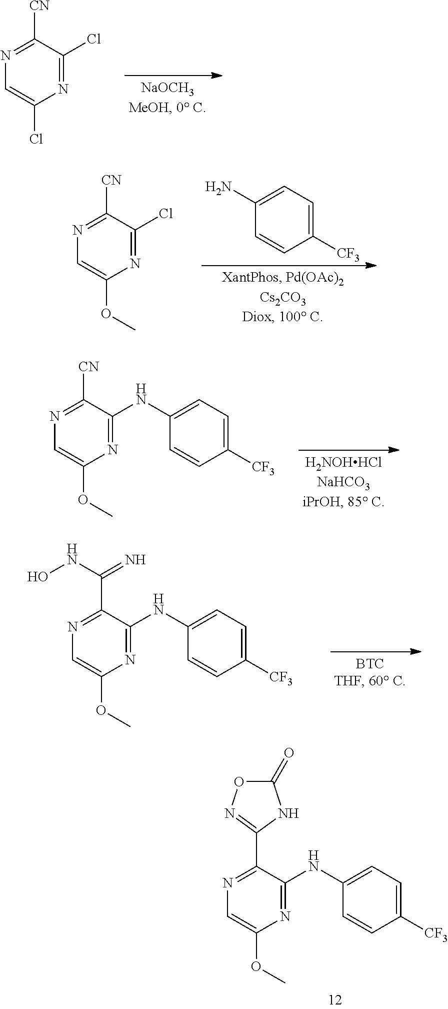 [SMILES: N#Cc1ncc(Cl)nc1Cl ; COc1cnc(C#N)c(Cl)n1 ; Nc1ccc(C(F)(F)F)cc1 ; COc1cnc(C#N)c(Nc2ccc(C(F)(F)F)cc2)n1 ; COc1cnc(C(=N)NO)c(Nc2ccc(C(F)(F)F)cc2)n1 ; COc1cnc(-c2noc(=O)[nH]2)c(Nc2ccc(C(F)(F)F)cc2)n1]

### Step 1: Preparation of 3-chloro-5-methoxy-pyrazine-2-carbonitrile

To a solution of 3,5-dichloropyrazine-2-carbonitrile (200 mg; 1.09 mmol) in MeOH (10 mL) cooled to 0° C. was added sodium methoxide (60 mg; 1.09 mmol). The solution was stirred for 2 h and was allowed to warm up to rt for 1 h. The reaction solution was concentrated to dryness. The residue was dissolved in a mixture of EA (60 ml) and water (60 mL). The organic layer was separated and washed with brine, dried over Na₂SO₄, filtered and concentrated. The residue was purified by column chromatography (silica gel; PE:EA; 8:1; v/v) to afford 3-chloro-5-methoxy-pyrazine-2-carbonitrile (110 mg) as a colorless oil.

MS m/z (+ESI): 170.0 [M+H]⁺.

¹H-NMR (400 MHz, DMSO-d₆) δ ppm: 8.50 (s, 1H), 4.02 (s, 3H).

### Step 2: Preparation of 5-methoxy-3-[4-(trifluoromethyl)anilino]pyrazine-2-carbonitrile

The title compound was prepared as a yellow solid following scheme 1 and in analogy to Example 1 (step 1) using 3-chloro-5-methoxy-pyrazine-2-carbonitrile and 4-(trifluoromethyl)aniline as starting materials at a temperature of 100° C. and after purification by column chromatography (silica gel; PE:EA; 8:1; v/v).

MS m/z (+ESI): 295.1 [M+H]⁺.

¹H-NMR (400 MHz, DMSO-d₆) δ ppm: 9.90 (s, 1H), 7.88 (s, 1H), 7.84 (d, J=8.6 Hz, 2H), 7.68 (d, J=8.6 Hz, 2H), 3.91 (s, 3H).

### Step 3: Preparation of N-hydroxy-5-methoxy-3-[4-(trifluoromethyl)anilino]pyrazine-2-carboxamidine

The title compound was prepared as a yellow solid following scheme 1 and in analogy to Example 1 (step 2) using 5-methoxy-3-[4-(trifluoromethyl)anilino]pyrazine-2-carbonitrile as starting material.

MS m/z (+ESI): 328.1 [M+H]⁺.

¹H-NMR (400 MHz, DMSO-d₆) δ ppm: 11.65 (s, 1H), 10.22 (s, 1H), 7.87 (d, J=8.8 Hz, 2H), 7.75 (s, 1H), 7.72 (d, J=8.8 Hz, 2H), 6.09 (s, 2H), 3.98 (s, 3H).

### Step 4: Preparation of 3-[5-methoxy-3-[4-(trifluoromethyl)anilino]pyrazin-2-yl]-4H-1,2,4-oxadiazol-5-one

The title compound was prepared as a yellow solid following scheme 1 and in analogy to Example 9 (step 3) using N-hydroxy-5-methoxy-3-[4-(trifluoromethyl)anilino]pyrazine-2-carboxamidine as starting material and after purification by preparative HPLC.

### Preparation of Example 14: 3-[3-[4-(cyclopropylmethoxy)anilino]pyrazin-2-yl]-4H-1,2,4-oxadiazol-5-one

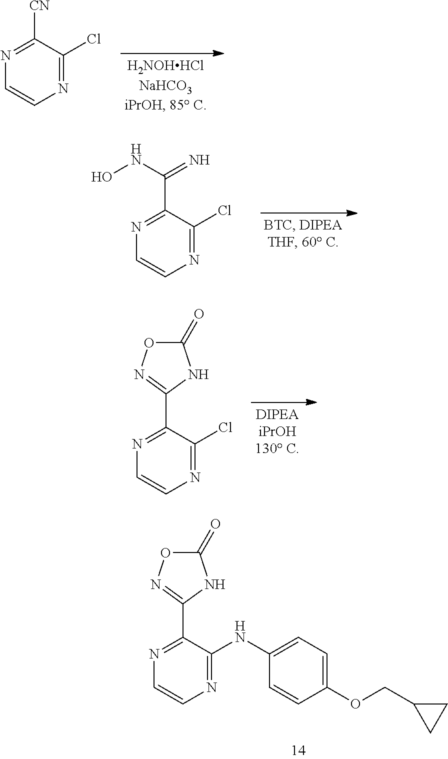 [SMILES: N#Cc1nccnc1Cl ; N=C(NO)c1nccnc1Cl ; O=c1[nH]c(-c2nccnc2Cl)no1 ; O=c1[nH]c(-c2nccnc2Nc2ccc(OCC3CC3)cc2)no1]

### Step 1: Preparation of 3-chloro-N-hydroxy-pyrazine-2-carboxamidine

The title compound was prepared as a yellow solid following scheme 1 and in analogy to Example 1 (step 2) using 3-chloropyrazine-2-carbonitrile as starting material.

MS m/z (+ESI): 173.1, 175.1 [M+H]⁺.

¹H-NMR (400 MHz, DMSO-d₆) δ ppm: 10.0 (s, 1H), 8.69 (d, J=2.6 Hz, 1H), 8.54 (d, J=2.6 Hz, 1H), 5.98 (br, 2H).

### Step 2: Preparation of 3-(3-chloropyrazin-2-yl)-4H-1,2,4-oxadiazol-5-one

To a solution of 3-chloro-N-hydroxy-pyrazine-2-carboxamidine (2 000 mg; 10.2 mmol) in THF (20 mL) was added DIPEA (5.1 mL; 30.6 mmol) and BTC (3 088 mg; 10.2 mmol). The reaction solution was stirred for 16 h. EA and water were added and the organic phase was separated, dried over Na₂SO₄, filtered and concentrated. The residue was purified by column chromatography (silica gel, PE:EA; 3:7 gradient to 0:1; v/v) to afford 3-(3-chloropyrazin-2-yl)-4H-1,2,4-oxadiazol-5-one (1 100 mg) as a yellow solid.

MS m/z (+ESI): 199.0, 201.1 [M+H]⁺.

### Step 3: Preparation of 3-[3-[4-(cyclopropylmethoxy)anilino]pyrazin-2-yl]-4H-1,2,4-oxadiazol-5-one

To a solution of 3-(3-chloropyrazin-2-yl)-4H-1,2,4-oxadiazol-5-one (100 mg; 0.48 mmol) and 4-(cyclopropylmethoxy)aniline (164 mg; 0.96 mmol) in i-PrOH (1 mL) was added DIPEA (0.24 mL; 1.44 mmol). The reaction solution was stirred under microwave at 130° C. for 0.5 h. After cooling down to rt, the solution was directly submitted to purification by preparative HPLC to afford 3-[3-[4-(cyclopropylmethoxy)anilino]pyrazin-2-yl]-4H-1,2,4-oxadiazol-5-one (20 mg) as a yellow solid.

### Preparation of Example 18: 3-[3-(4-thiazol-2-ylanilino)pyrazin-2-yl]-4H-1,2,4-oxadiazol-5-one

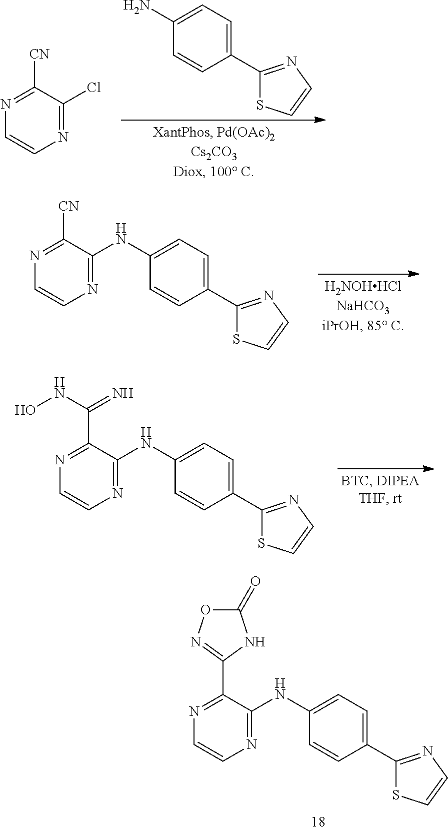

### Step 1: Preparation of 3-(4-thiazol-2-ylanilino)pyrazine-2-carbonitrile

The title compound was prepared as a yellow solid following scheme 1 and in analogy to Example 1 (step 1) using 3-chloropyrazine-2-carbonitrile and 4-thiazol-2-ylaniline as starting materials at a temperature of 100° C. and after purification by column chromatography (silica gel; PE:EA; 1:0 gradient to 1:1; v/v).

MS m/z (+ESI): 280.1 [M+H]⁺.

### Step 2: Preparation of N-hydroxy-3-(4-thiazol-2-ylanilino)pyrazine-2-carboxamidine

The title compound was prepared as a yellow solid following scheme 1 and in analogy to Example 1 (step 2) using 3-(4-thiazol-2-ylanilino)pyrazine-2-carbonitrile as starting material.

MS m/z (+ESI): 313.1 [M+H]⁺.

### Step 3: Preparation of 3-[3-(4-thiazol-2-ylanilino)pyrazin-2-yl]-4H-1,2,4-oxadiazol-5-one

To a solution of N-hydroxy-3-(4-thiazol-2-ylanilino)pyrazine-2-carboxamidine (200 mg; 0.58 mmol) and DIPEA (0.29 mL; 1.73 mmol) in THF (10 mL) was added BTC (175 mg; 0.58 mmol). The reaction solution was stirred for 16 h. The solution was concentrated and the residue was purified by preparative HPLC to afford 3-[3-(4-thiazol-2-ylanilino)pyrazin-2-yl]-4H-1,2,4-oxadiazol-5-one (147 mg) as a yellow solid.

### Preparation of Example 26: 3-[4,6-dimethyl-2-[4-(trifluoromethyl)anilino]-3-pyridyl]-4H-1,2,4-oxadiazol-5-one

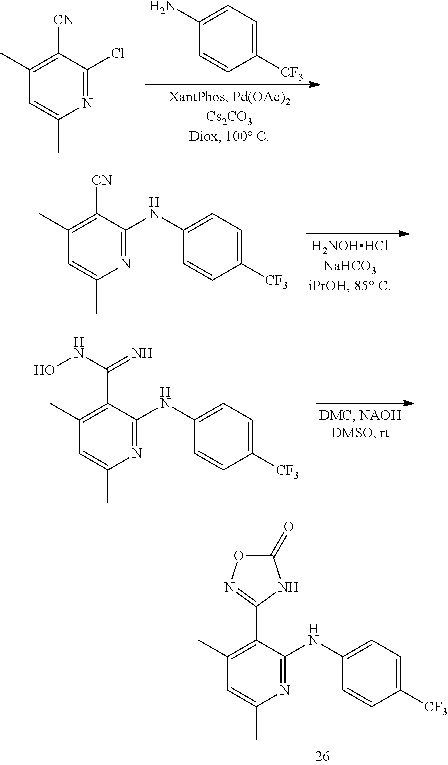

### Step 1: Preparation of 4,6-dimethyl-2-[4-(trifluoromethyl)anilino]pyridine-3-carbonitrile

The title compound was prepared as a yellow solid following scheme 1 and in analogy to Example 1 (step 1) using 2-chloro-4,6-dimethyl-pyridine-3-carbonitrile and 4-(trifluoromethyl)aniline as starting materials at a temperature of 100° C. and after purification by column chromatography (silica gel; PE:EA; 6:1; v/v).

MS m/z (+ESI): 292.2 [M+H]⁺.

¹H-NMR (400 MHz, DMSO-d₆) δ ppm: 9.34 (s, 1H), 7.80 (d, J=8.6 Hz, 2H), 7.61 (d, J=8.6 Hz, 2H), 6.87 (s, 1H), 2.40 (s, 3H), 2.38 (s, 3H).

### Step 2: Preparation of N-hydroxy-4,6-dimethyl-2-[4-(trifluoromethyl)anilino]pyridine-3-carboxamidine

The title compound was prepared as a white solid following scheme 1 and in analogy to Example 1 (step 2) using 4,6-dimethyl-2-[4-(trifluoromethyl)anilino]pyridine-3-carbonitrile as starting material and after purification by column chromatography (silica gel; PE:EA; 4:1; v/v).

MS m/z (+ESI): 325.2 [M+H]⁺.

¹H-NMR (400 MHz, DMSO-d₆) δ ppm: 9.59 (s, 1H), 8.39 (s, 1H), 7.82 (d, J=8.6 Hz, 2H), 7.58 (d, J=8.6 Hz, 2H), 6.70 (s, 1H), 6.04 (br, 2H), 2.36 (s, 3H), 2.25 (s, 3H).

### Step 3: Preparation of 3-[4,6-dimethyl-2-[4-(trifluoromethyl)anilino]-3-pyridyl]-4H-1,2,4-oxadiazol-5-one

A suspension of N-hydroxy-4,6-dimethyl-2-[4-(trifluoromethyl)anilino]pyridine-3-carboxamidine (200 mg; 0.56 mmol), DMC (0.05 mL; 0.56 mmol) and NaOH (68 mg; 1.67 mmol) in DMSO (8 mL) was stirred for 4 h. The suspension was filtered and the solid was washed with EA. The filtrate was directly purified by preparative HPLC to afford 3-[4,6-dimethyl-2-[4-(trifluoromethyl)anilino]-3-pyridyl]-4H-1,2,4-oxadiazol-5-one (72 mg) as a white solid.

### Preparation of Example 27: N-[4-[[3-(5-oxo-4H-1,2,4-oxadiazol-3-yl)pyrazin-2-yl]amino]phenyl]cyclobutanecarboxamide

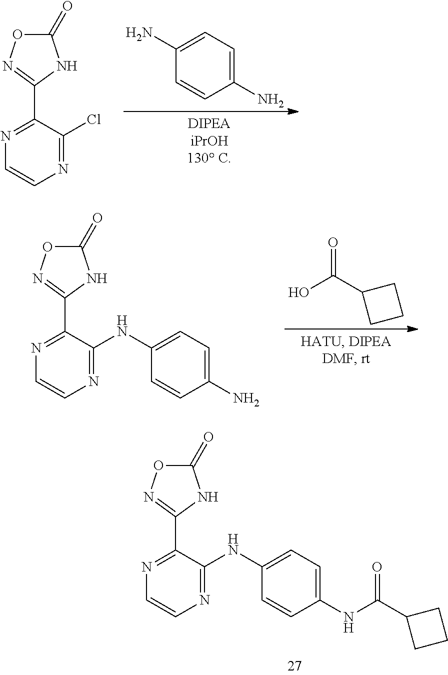 [SMILES: O=c1[nH]c(-c2nccnc2Cl)no1 ; Nc1ccc(N)cc1 ; Nc1ccc(Nc2nccnc2-c2noc(=O)[nH]2)cc1 ; O=C(O)C1CCC1 ; O=C(Nc1ccc(Nc2nccnc2-c2noc(=O)[nH]2)cc1)C1CCC1]

### Step 1: Preparation of 3-[3-(4-aminoanilino)pyrazin-2-yl]-4H-1,2,4-oxadiazol-5-one

The title compound was prepared as a yellow solid following scheme 2 and in analogy to Example 14 (step 3) using 3-(3-chloropyrazin-2-yl)-4H-1,2,4-oxadiazol-5-one and benzene-1,4-diamine as starting materials and after purification by preparative HPLC.

MS m/z (+ESI): 271.1 [M+H]⁺.

### Step 2: Preparation of N-[4-[[3-(5-oxo-4H-1,2,4-oxadiazol-3-yl)pyrazin-2-yl]amino]phenyl]cyclobutanecarboxamide

To a solution of 3-[3-(4-aminoanilino)pyrazin-2-yl]-4H-1,2,4-oxadiazol-5-one (100 mg; 0.33 mmol) in DMF (6 mL) was added HATU (194 mg; 0.50 mmol), DIPEA (0.17 mL; 1.00 mmol) and cyclobutanecarboxylic (0.05 mL; 0.50 mmol). The reaction solution was stirred for 4 h. The reaction solution was directly purified by preparative HPLC to afford N-[4-[[3-(5-oxo-4H-1,2,4-oxadiazol-3-yl)pyrazin-2-yl]amino]phenyl]cyclobutanecarboxamide (39 mg) as a yellow solid.

### Preparation of Example 31: 3-[5-methyl-3-[4-(trifluoromethyl)anilino]pyrazin-2-yl]-4H-1,2,4-oxadiazol-5-one

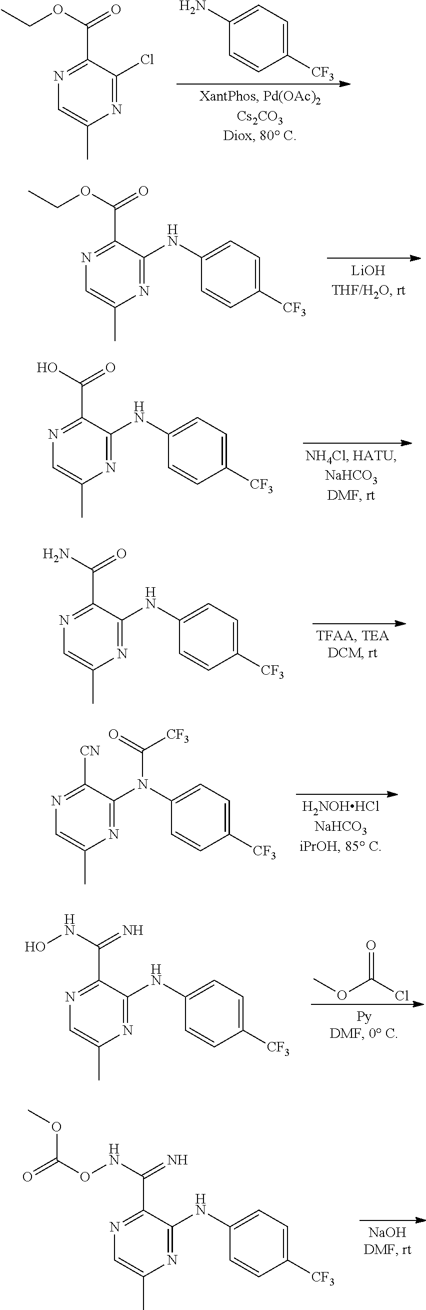

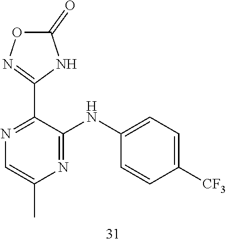 [SMILES: Cc1cnc(-c2noc(=O)[nH]2)c(Nc2ccc(C(F)(F)F)cc2)n1]

### Step 1: Preparation of ethyl 5-methyl-3-[4-(trifluoromethyl)anilino]pyrazine-2-carboxylate

The title compound was prepared as a yellow solid following scheme 1 and in analogy to Example 1 (step 1) using ethyl 3-chloro-5-methyl-pyrazine-2-carboxylate and 4-(trifluoromethyl)aniline as starting materials and after purification by column chromatography (silica gel; PE:EA; 5:1; v/v).

MS m/z (+ESI): 326.1 [M+H]⁺.

¹H-NMR (400 MHz, CDCl₃) δ ppm: 10.52 (s, 1H), 8.05 (s, 1H), 7.88 (d, J=8.6 Hz, 2H), 7.61 (d, J=8.6 Hz, 2H), 4.52 (q, J=7.2 Hz, 2H), 2.57 (s, 3H), 1.49 (t, J=7.2 Hz, 3H).

### Step 2: Preparation of 5-methyl-3-[4-(trifluoromethyl)anilino]pyrazine-2-carboxylic acid

To a solution of ethyl 5-methyl-3-[4-(trifluoromethyl)anilino]pyrazine-2-carboxylate (350 mg; 1.02 mmol) in THF (10 mL) was added a solution of LiOH (371 mg; 15.33 mmol) in water (2 mL). The solution was stirred for 2 h. THF was removed under reduced pressure. The aqueous layer was acidified to pH 2 with HCl solution, 3N in water and the product was extracted with EA. The organic layer was washed with brine, dried over Na₂SO₄, filtered and concentrated to afford 5-methyl-3-[4-(trifluoromethyl)anilino]pyrazine-2-carboxylic acid (300 mg) as a yellow oil.

MS m/z (+ESI): 298.1 [M+H]⁺.

### Step 3: Preparation of 5-methyl-3-[4-(trifluoromethyl)anilino]pyrazine-2-carboxamide

To a solution of 5-methyl-3-[4-(trifluoromethyl)anilino]pyrazine-2-carboxylic acid (300 mg; 0.96 mmol) in DMF (10 mL) was added NH₄Cl (78 mg; 1.44 mmol), NaHCO₃ (163 mg; 1.92 mmol) and HATU (564 mg; 1.44 mmol). The suspension was stirred for 2 h. The suspension was concentrated under reduced pressure. The residue was purified by column chromatography (silica gel; DCM:MeOH; 1:0 gradient to 4:1; v/v) to afford 5-methyl-3-[4-(trifluoromethyl)anilino]pyrazine-2-carboxamide (270 mg) as a yellow solid.

MS m/z (+ESI): 297.1 [M+H]⁺.

¹H-NMR (400 MHz, CDCl₃) δ ppm: 11.18 (s, 1H), 7.90 (d, J=8.6 Hz, 2H), 7.85 (br, 2H), 7.59 (d, J=8.6 Hz, 2H), 5.57 (br, 1H), 2.56 (s, 3H).

### Step 4: Preparation of N-(3-cyano-6-methyl-pyrazin-2-yl)-2,2,2-trifluoro-N-[4-(trifluoromethyl)phenyl]acetamide

To a solution of 5-methyl-3-[4-(trifluoromethyl)anilino]pyrazine-2-carboxamide (270 mg; 0.87 mmol) in DCM (5 mL) was added TFAA (0.30 mL; 2.16 mmol) and TEA (0.30 mL; 2.16 mmol). The solution was stirred for 2 h. The solution was concentrated under reduced pressure. The residue was purified by column chromatography (silica gel; PE:EA; 5:1; v/v) to afford N-(3-cyano-6-methyl-pyrazin-2-yl)-2,2,2-trifluoro-N-[4-(trifluoromethyl)phenyl]acetamide (260 mg) as a yellow oil.

MS m/z (+ESI): 375.0 [M+H]⁺.

¹H-NMR (400 MHz, CDCl₃) δ ppm: 8.61 (s, 1H), 7.77-7.68 (m, 4H), 2.71 (s, 3H).

### Step 5: Preparation of N-hydroxy-5-methyl-3-[4-(trifluoromethyl)anilino]pyrazine-2-carboxamidine

The title compound was prepared as a yellow solid following scheme 1 and in analogy to Example 1 (step 2) using N-(3-cyano-6-methyl-pyrazin-2-yl)-2,2,2-trifluoro-N-[4-(trifluoromethyl)phenyl]acetamide as starting material.

MS m/z (+ESI): 312.1 [M+H]⁺.

¹H-NMR (400 MHz, CDCl₃) δ ppm: 10.61 (s, 1H), 7.90-7.85 (m, 3H), 7.57 (d, J=8.8 Hz, 2H), 5.80 (br, 2H), 2.52 (s, 3H).

### Step 6: Preparation of methyl [[5-methyl-3-[4-(trifluoromethyl)anilino]pyrazine-2-carboximidoyl]amino]carbonate

To a solution of N-hydroxy-5-methyl-3-[4-(trifluoromethyl)anilino]pyrazine-2-carboxamidine (20 mg; 0.06 mmol) in DMF (3 mL) cooled to 0° C. was added pyridine (0.006 mL; 0.07 mmol) and methyl chloroformate (0.006 mL; 0.07 mmol). The solution was allowed to warm up to rt and stirred for 3 h. The solution was concentrated under reduced pressure. The residue was purified by column chromatography (silica gel; PE:EA; 5:1; v/v) to afford methyl [[5-methyl-3-[4-(trifluoromethyl)anilino]pyrazine-2-carboximidoyl]amino] carbonate (14 mg) as a white solid.

MS m/z (+ESI): 369.8 [M+H]⁺.

### Step 7: Preparation of 3-[5-methyl-3-[4-(trifluoromethyl)anilino]pyrazin-2-yl]-4H-1,2,4-oxadiazol-5-one

To a solution of methyl [[5-methyl-3-[4-(trifluoromethyl)anilino]pyrazine-2-carboximidoyl]amino]carbonate (80 mg; 0.20 mmol) in DMF (10 mL) was added NaOH (10 mg; 0.23 mmol) and the solution was stirred for 3 h. The solution was concentrated under reduced pressure. The residue was purified by preparative HPLC to afford 3-[5-methyl-3-[4-(trifluoromethyl)anilino]pyrazin-2-yl]-4H-1,2,4-oxadiazol-5-one (55 mg) as a white solid.

### Preparation of Example 33: 3-[3-[3-amino-4-(trifluoromethyl)anilino]pyrazin-2-yl]-4H-1,2,4-oxadiazol-5-one

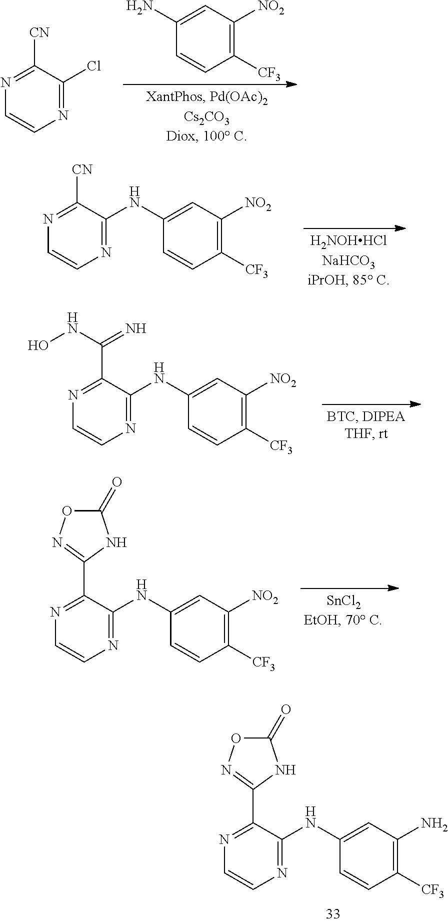

### Step 1: Preparation of 3-[3-nitro-4-(trifluoromethyl)anilino]pyrazine-2-carbonitrile

The title compound was prepared as a yellow solid following scheme 1 and in analogy to Example 1 (step 1) using 3-chloropyrazine-2-carbonitrile and 3-nitro-4-(trifluoromethyl)benzenamine as starting materials at a temperature of 100° C. and after purification by column chromatography (silica gel; PE:EA; 1:0 gradient to 3:2; v/v).

MS m/z (+ESI): 310.1 [M+H]⁺.

¹H-NMR (400 MHz, DMSO-d₆) δ ppm: 10.36 (s, 1H), 8.60 (d, J=2.4 Hz, 1H), 8.41 (d, J=2.0 Hz, 1H), 8.38 (d, J=2.4 Hz, 1H), 8.10 (dd, J=8.8, 1.2 Hz, 1H), 7.98 (d, J=8.8 Hz, 1H).

### Step 2: Preparation of N-hydroxy-3-[3-nitro-4-(trifluoromethyl)anilino]pyrazine-2-carboxamidine

The title compound was prepared as a yellow solid following scheme 1 and in analogy to Example 1 (step 2) using 3-[3-nitro-4-(trifluoromethyl)anilino]pyrazine-2-carbonitrile as starting material and after purification by column chromatography (silica gel; PE:EA; 1:0 gradient to 3:2; v/v).

MS m/z (+ESI): 343.1 [M+H]⁺.

¹H-NMR (400 MHz, DMSO-d₆) δ ppm: 12.08 (s, 1H), 10.62 (s, 1H), 8.56 (s, 1H), 8.36 (d, J=2.4 Hz, 1H), 8.23 (d, J=2.4 Hz, 1H), 8.01-7.99 (m, 2H), 6.32 (br, 2H).

### Step 3: Preparation of 3-[3-[3-nitro-4-(trifluoromethyl)anilino]pyrazin-2-yl]-4H-1,2,4-oxadiazol-5-one

The title compound was prepared as a yellow solid following scheme 1 and in analogy to Example 18 (step 3) using N-hydroxy-3-[3-nitro-4-(trifluoromethyl)anilino]pyrazine-2-carboxamidine as starting material and after purification by column chromatography (silica gel; PE:EA; 1:0 gradient to 3:2; v/v).

MS m/z (+ESI): 369.1 [M+H]⁺.

¹H-NMR (400 MHz, DMSO-d₆) δ ppm: 13.43 (br, 1H), 9.61 (s, 1H), 8.59-8.56 (m, 2H), 8.42 (s, 1H), 8.13-8.10 (m, 1H), 7.98 (d, J=8.8 Hz, 1H).

### Step 4: Preparation of 3-[3-[3-amino-4-(trifluoromethyl)anilino]pyrazin-2-yl]-4H-1,2,4-oxadiazol-5-one

A solution of 3-[3-[3-nitro-4-(trifluoromethyl)anilino]pyrazin-2-yl]-4H-1,2,4-oxadiazol-5-one (75 mg; 0.17 mmol), Tin (II) chloride, dihydrate (120 mg; 0.52 mmol) in EtOH (10 mL) was heated to 70° C. and stirred for 1 h. The mixture was evaporated. The residue was purified by preparative HPLC to afford 3-[3-[3-amino-4-(trifluoromethyl)anilino]pyrazin-2-yl]-4H-1,2,4-oxadiazol-5-one (23 mg) as a yellow solid.

### Preparation of Example 35: N-[6-(5-oxo-4H-1,2,4-oxadiazol-3-yl)-5-[4-(trifluoromethyl)anilino]pyrazin-2-yl]acetamide

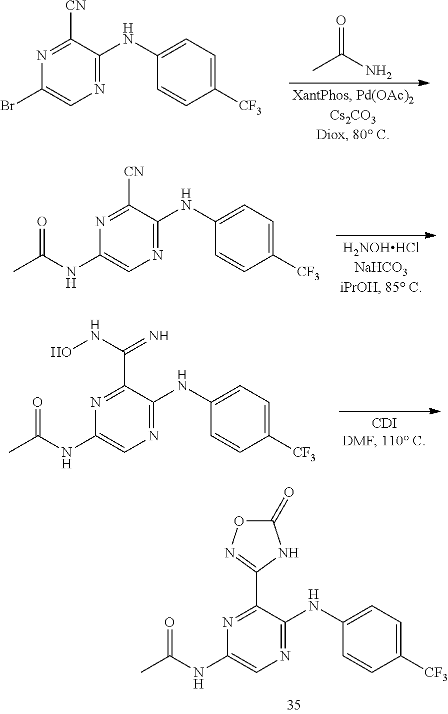

### Step 1: Preparation of N-[6-cyano-5-[4-(trifluoromethyl)anilino]pyrazin-2-yl]acetamide

The title compound was prepared as a yellow solid following scheme 1 and in analogy to Example 1 (step 1) using 6-bromo-3-[4-(trifluoromethyl)anilino]pyrazine-2-carbonitrile (intermediate Example 5 step 1) and acetamide (3 equivalents) as starting materials and after purification by column chromatography (silica gel; PE:EA; 2:1; v/v).

MS m/z (+ESI): 322.1 [M+H]⁺.

¹H-NMR (400 MHz, CDCl₃) δ ppm: 9.39 (s, 1H), 7.72-7.69 (m, 3H), 7.64 (d, J=8.8 Hz, 2H), 7.08 (s, 1H), 2.27 (s, 3H).

### Step 2: Preparation of N-[6-(N-hydroxycarbamimidoyl)-5-[4-(trifluoromethyl)anilino]pyrazin-2-yl]acetamide

The title compound was prepared as a brown solid following scheme 1 and in analogy to Example 1 (step 2) using N-[6-cyano-5-[4-(trifluoromethyl)anilino]pyrazin-2-yl]acetamide as starting material.

MS m/z (+ESI): 355.1 [M+H]⁺.

¹H-NMR (400 MHz, CDCl₃) δ ppm: 11.25 (s, 1H), 10.63 (s, 1H), 10.47 (s, 1H), 8.93 (s, 1H), 7.84 (d, J=8.8 Hz, 2H), 7.68 (d, J=8.8 Hz, 2H), 6.14 (br, 2H), 2.11 (s, 3H).

### Step 3: Preparation of N-[6-(5-oxo-4H-1,2,4-oxadiazol-3-yl)-5-[4-(trifluoromethyl)anilino]pyrazin-2-yl]acetamide

The title compound was prepared as a yellow solid following scheme 1 and in analogy to Example 2 (step 3) using N-[6-(N-hydroxycarbamimidoyl)-5-[4-(trifluoromethyl)anilino]pyrazin-2-yl]acetamide as starting material and after purification by preparative HPLC.

### Preparation of Example 36: 3-[5-amino-3-[4-(trifluoromethyl)anilino]pyrazin-2-yl]-4H-1,2,4-oxadiazol-5-one

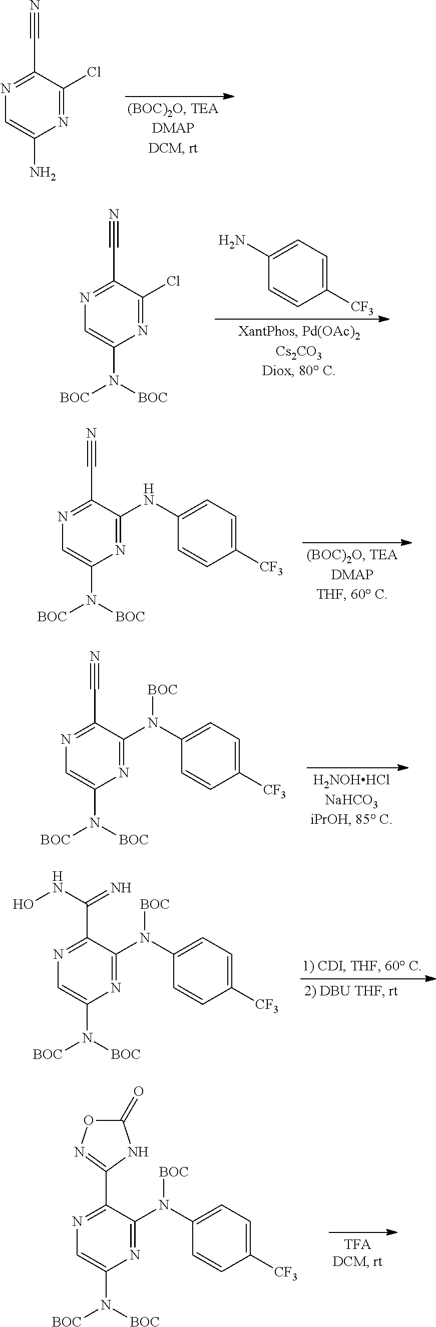

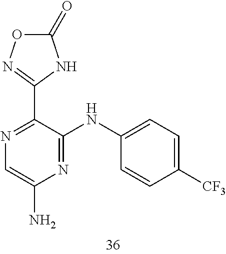 [SMILES: Nc1cnc(-c2noc(=O)[nH]2)c(Nc2ccc(C(F)(F)F)cc2)n1]

### Step 1: Preparation of tert-butyl N-tert-butoxycarbonyl-N-(6-chloro-5-cyano-pyrazin-2-yl)carbamate

To a solution of 5-amino-3-chloro-pyrazine-2-carbonitrile (4 000 mg; 12.94 mmol) in DCM (40 mL) cooled to 0° C. were added (BOC)₂O (7 205 mg; 32.35 mmol), TEA (5.47 mL; 38.82 mmol) and DMAP (160 mg; 1.29 mmol). The solution was stirred for 12 h at rt and then concentrated under reduced pressure. The residue was purified by column chromatography (silica gel; PE:EA; 1:0 gradient to 4:1, v/v) to afford tert-butyl N-tert-butoxycarbonyl-N-(6-chloro-5-cyano-pyrazin-2-yl)carbamate (3 900 mg) as an off-white solid.

¹H-NMR (400 MHz, DMSO-d₆) δ ppm: 9.04 (s, 1H), 1.44 (s, 18H).

### Step 2: Preparation of tert-butyl N-tert-butoxycarbonyl-N-[5-cyano-6-[4-(trifluoromethyl)anilino]pyrazin-2-yl]carbamate

The title compound was prepared as a white solid following scheme 1 and in analogy to Example 1 (step 1) using tert-butyl N-tert-butoxycarbonyl-N-(6-chloro-5-cyano-pyrazin-2-yl)carbamate and 4-(trifluoromethyl)aniline as starting materials and after purification by column chromatography (silica gel; PE:EA; 1:0 gradient to 7:3; v/v).

MS m/z (+ESI): 480.2 [M+H]⁺.

### Step 3: Preparation of tert-butyl N-[6-[bis(tert-butoxycarbonyl)amino]-3-cyano-pyrazin-2-yl]-N-[4-(trifluoromethyl)phenyl]carbamate

To a solution of tert-butyl N-tert-butoxycarbonyl-N-[5-cyano-6-[4-(trifluoromethyl)anilino]pyrazin-2-yl]carbamate (730 mg; 1.45 mmol) in THF (10 mL) were added (BOC)₂O (387 mg; 1.74 mmol), TEA (0.20 mL; 1.45 mmol) and DMAP (268 mg; 2.17 mmol). The solution was heated to 60° C. and stirred for 1.5 h. The solution was concentrated under reduced pressure. The residue was purified by column chromatography (silica gel; PE:EA; 1:0 gradient to 4:1; v/v) to afford tert-butyl N-[6-[bis(tert-butoxycarbonyl)amino]-3-cyano-pyrazin-2-yl]-N-[4-(trifluoromethyl)phenyl]carbamate (750 mg) as a white solid.

MS m/z (+ESI): 580.3 [M+H]⁺.

¹H-NMR (400 MHz, DMSO-d₆) δ ppm: 9.01 (s, 1H), 7.76 (d, J=8.6 Hz, 2H), 7.56 (d, J=8.6 Hz, 2H), 1.45 (s, 9H). 1.28 (s, 18H).

### Step 4: Preparation of tert-butyl N-[6-[bis(tert-butoxycarbonyl)amino]-3-(N-hydroxycarbamimidoyl)pyrazin-2-yl]-N-[4-(trifluoromethyl)phenyl]carbamate

The title compound was prepared as a yellow solid following scheme 1 and in analogy to Example 1 (step 2) using tert-butyl N-[6-[bis(tert-butoxycarbonyl)amino]-3-cyano-pyrazin-2-yl]-N-[4-(trifluoromethyl)phenyl]carbamate as starting material.

MS m/z (+ESI): 613.3 [M+H]⁺.

### Step 5: Preparation of tert-butyl N-[6-[bis(tert-butoxycarbonyl)amino]-3-(5-oxo-4H-1,2,4-oxadiazol-3-yl)pyrazin-2-yl]-N-[4-(trifluoromethyl)phenyl]carbamate

To a solution of tert-butyl N-[6-[bis(tert-butoxycarbonyl)amino]-3-(N-hydroxycarbamimidoyl)pyrazin-2-yl]-N-[4-(trifluoromethyl)phenyl]carbamate (300 mg; 0.39 mmol) in THF (3 mL) was added CDI (195 mg; 1.18 mmol). The solution was heated to 60° C. and stirred for 2 h. Then, the solution was cooled to 25° C. and DBU (0.18 mL; 1.18 mmol) was added. After stirring for 1 h, the solution was concentrated under reduced pressure. The residue was dissolved in EA and the organic solution was washed with saturated aqueous citric acid solution, followed by brine solution, dried over Na₂SO₄, filtered and concentrated to afford tert-butyl N-[6-[bis(tert-butoxycarbonyl)amino]-3-(5-oxo-4H-1,2,4-oxadiazol-3-yl)pyrazin-2-yl]-N-[4-(trifluoromethyl)phenyl]carbamate (390 mg) as a light yellow waxy solid.

### Step 6: Preparation of 3-[5-amino-3-[4-(trifluoromethyl)anilino]pyrazin-2-yl]-4H-1,2,4-oxadiazol-5-one

The title compound was prepared as a yellow solid following scheme 1 and in analogy to Example 4 (step 5) using tert-butyl N-[6-[bis(tert-butoxycarbonyl)amino]-3-(5-oxo-4H-1,2,4-oxadiazol-3-yl)pyrazin-2-yl]-N-[4-(trifluoromethyl)phenyl]carbamate as starting material and after purification by preparative HPLC.

### Preparation of Example 37: 3-[3-[4-(trifluoromethyl)anilino]quinoxalin-2-yl]-4H-1,2,4-oxadiazol-5-one

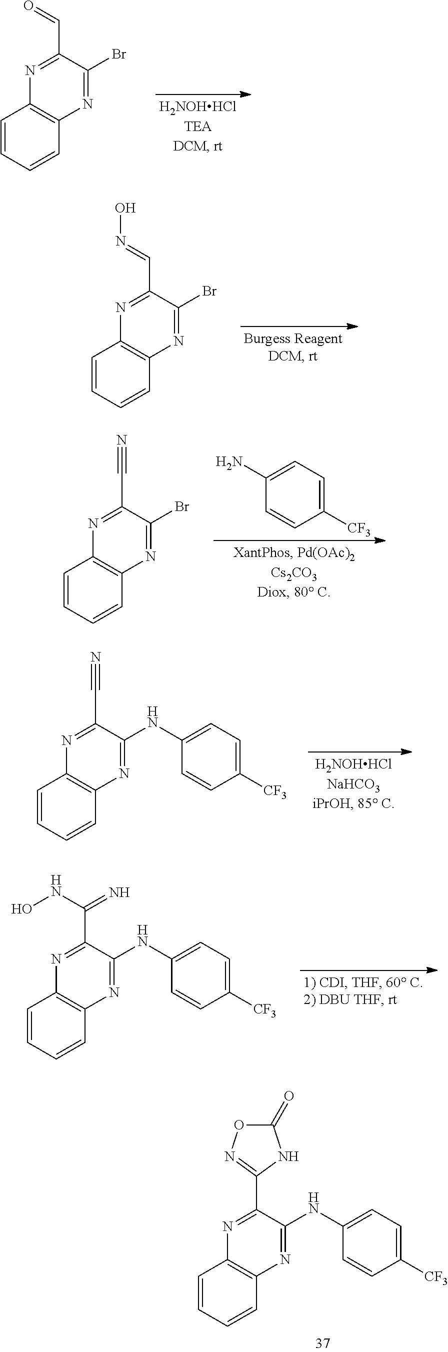

### Step 1: Preparation of 3-bromoquinoxaline-2-carbaldehyde oxime

To a solution of 3-bromoquinoxaline-2-carbaldehyde (300 mg; 1.20 mmol) in DCM (10 mL) were added TEA (1.01 mL; 7.21 mmol) and NH₂OH.HCl (255 mg; 3.61 mmol). The suspension was stirred for 20 h. H₂O (30 mL) was added and the mixture was extracted with DCM (4×40 mL). The combined organic layers were washed with brine, dried over Na₂SO₄, filtered and concentrated to afford 3-bromoquinoxaline-2-carbaldehyde oxime (310 mg) as a yellow solid.

MS m/z (+ESI): 252.0, 254.0 [M+H]⁺.

### Step 2: Preparation of 3-bromoquinoxaline-2-carbonitrile

To a suspension of 3-bromoquinoxaline-2-carbaldehyde oxime (100 mg; 0.36 mmol) in DCM (5 mL) was added Burgess Reagent (351 mg; 1.43 mmol) in three portions over 1 h. The suspension was then stirred for 40 h. The reaction solution was concentrated to dryness. The residue was purified by column chromatography (silica gel; PE:EA; 9:1; v/v) to afford 3-bromoquinoxaline-2-carbonitrile (48 mg) as a white solid.

MS m/z (+ESI): 234.0, 236.0 [M+H]⁺.

### Step 3: Preparation of 3-[4-(trifluoromethyl)anilino]quinoxaline-2-carbonitrile

The title compound was prepared as a yellow solid following scheme 1 and in analogy to Example 1 (step 1) using 3-bromoquinoxaline-2-carbonitrile and 4-(trifluoromethyl)aniline as starting materials and after purification by column chromatography (silica gel; PE:EA; 1:0 gradient to 4:1; v/v).

MS m/z (+ESI): 315.2 [M+H]⁺.

### Step 4: Preparation of N-hydroxy-3-[4-(trifluoromethyl)anilino]quinoxaline-2-carboxamidine

The title compound was prepared as a yellow solid following scheme 1 and in analogy to Example 1 (step 2) using 3-[4-(trifluoromethyl)anilino]quinoxaline-2-carbonitrile as starting material.

MS m/z (+ESI): 348.1 [M+H]⁺.

### Step 5: Preparation of 3-[3-[4-(trifluoromethyl)anilino]quinoxalin-2-yl]-4H-1,2,4-oxadiazol-5-one

The title compound was prepared as a yellow solid following scheme 1 and in analogy to Example 36 (step 5) using N-hydroxy-3-[4-(trifluoromethyl)anilino]quinoxaline-2-carboxamidine as starting material and after purification by preparative HPLC.

### Preparation of Example 45: 3-[5-amino-2-[4-(trifluoromethyl)anilino]-3-pyridyl]-4H-1,2,4-oxadiazol-5-one

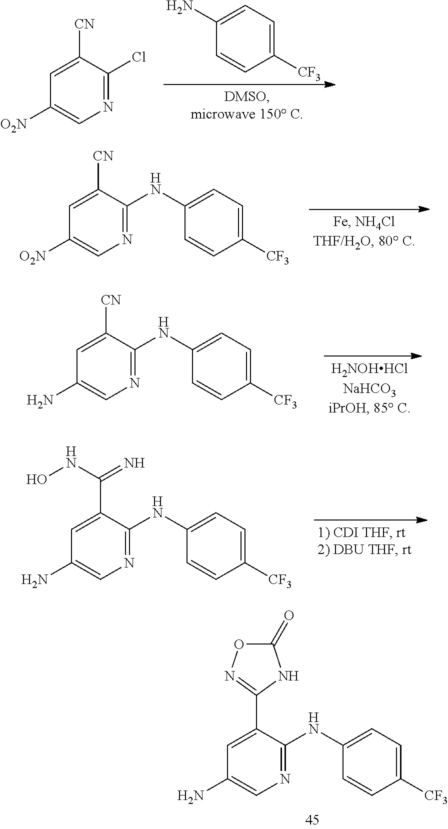

### Step 1: Preparation of 5-nitro-2-[4-(trifluoromethyl)anilino]pyridine-3-carbonitrile

A solution of 2-chloro-5-nitropyridine-3-carbonitrile (350 mg; 1.81 mmol) and 4-(trifluoromethyl)aniline (0.46 mL; 3.62 mmol) in DMSO (5 mL) was stirred at 150° C. under microwave for 0.5 h. The solution was diluted with EA (20 mL) and successively washed with H₂O (2×5 mL) and brine (2×5 mL), dried over Na₂SO₄, filtered and concentrated. The residue was purified by column chromatography (silica gel; PE:EA; 1:0 gradient to 4:1; v/v) to afford 5-nitro-2-[4-(trifluoromethyl)anilino]pyridine-3-carbonitrile (290 mg) as a yellow solid.

MS m/z (+ESI): 309.1 [M+H]⁺.

¹H-NMR (400 MHz, DMSO-d₆) δ ppm: 10.42 (s, 1H), 9.16 (d, J=2.6 Hz, 1H), 9.01 (d, J=2.6 Hz, 1H), 7.82 (d, J=8.8 Hz, 2H), 7.34 (d, J=8.8 Hz, 2H).

### Step 2: Preparation of 5-amino-2-[4-(trifluoromethyl)anilino]pyridine-3-carbonitrile

To a stirred solution of 5-nitro-2-[4-(trifluoromethyl)anilino]pyridine-3-carbonitrile (520 mg; 1.52 mmol) in THF (20 mL) and H₂O (5 mL) was added iron powder (1 731 mg; 30.4 mmol) and NH₄Cl (1 641 mg; 30.4 mmol). The suspension was stirred at 80° C. for 2.5 h. The suspension was filtered through a plug of Celite® and washed with EA (30 mL). The combined filtrate was washed with H₂O (20 mL) and brine (10 mL). The organic layer was dried over Na₂SO₄, filtered and concentrated. The residue was purified by column chromatography (silica gel; PE:EA; 1:0 gradient to 0:1; v/v) to afford 5-amino-2-[4-(trifluoromethyl)anilino]pyridine-3-carbonitrile (300 mg) as an orange solid.

MS m/z (+ESI): 279.1 [M+H]⁺.

¹H-NMR (400 MHz, DMSO-d₆) δ ppm: 9.00 (s, 1H), 7.95 (d, J=3.2 Hz, 1H), 7.51 (d, J=8.8 Hz, 2H), 7.40 (d, J=8.8 Hz, 2H), 7.30 (d, J=3.2 Hz, 1H), 5.41 (s, 2H).

### Step 3: Preparation of 5-amino-N-hydroxy-2-[4-(trifluoromethyl)anilino]pyridine-3-carboxamidine

The title compound was prepared as an orange solid following scheme 1 and in analogy to Example 1 (step 2) using 5-amino-2-[4-(trifluoromethyl)anilino]pyridine-3-carbonitrile as starting material.

MS m/z (+ESI): 312.2 [M+H]⁺.

### Step 4: Preparation of 3-[5-amino-2-[4-(trifluoromethyl)anilino]-3-pyridyl]-4H-1,2,4-oxadiazol-5-one

The title compound was prepared as a green solid following scheme 1 and in analogy to Example 1 (step 3) using 5-amino-N-hydroxy-2-[4-(trifluoromethyl)anilino]pyridine-3-carboxamidine as starting material and after purification by preparative HPLC.

### Preparation of Example 46: 3-[5-(methylamino)-2-[4-(trifluoromethyl)anilino]-3-pyridyl]-4H-1,2,4-oxadiazol-5-one

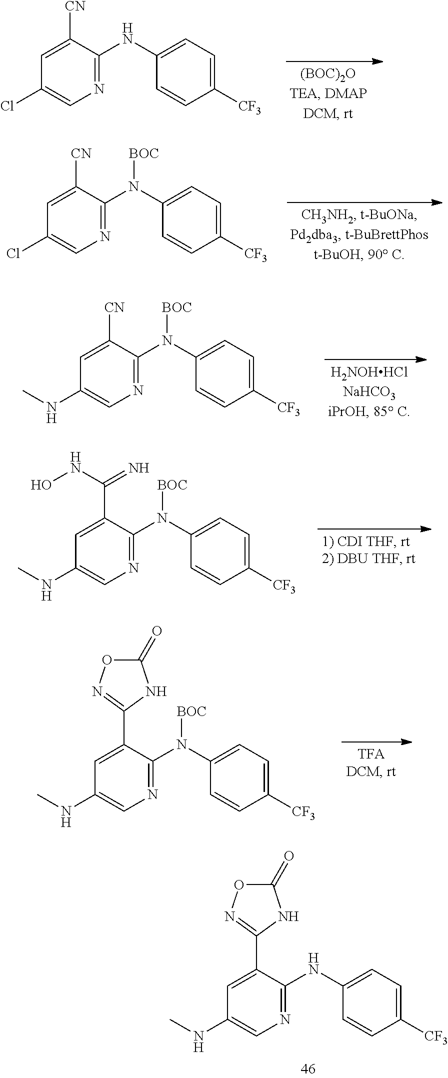

### Step 1: Preparation of tert-butyl N-(5-chloro-3-cyano-2-pyridyl)-N-[4-(trifluoromethyl)phenyl]carbamate

The title compound was prepared as an orange solid following scheme 1 and in analogy to Example 36 (step 1) using 5-chloro-2-[4-(trifluoromethyl)anilino]pyridine-3-carbonitrile (intermediate Ex. 44 step 1) as starting material.

MS m/z (+ESI): 342.1, 344.1 [M-(t-Bu)+H]⁺.

¹H-NMR (400 MHz, CDCl₃) δ ppm: 8.58 (d, J=2.4 Hz, 1H), 8.03 (d, J=2.4 Hz, 1H), 7.63 (d, J=8.4 Hz, 2H), 7.43 (d, J=8.4 Hz, 2H), 1.54 (s, 9H).

### Step 2: Preparation of tert-butyl N-[3-cyano-5-(methylamino)-2-pyridyl]-N-[4-(trifluoromethyl)phenyl]carbamate

To a solution of tert-butyl N-(5-chloro-3-cyano-2-pyridyl)-N-[4-(trifluoromethyl)phenyl]carbamate (1 200 mg; 2.41 mmol) and methylamine solution, 2M in THF (2.4 mL; 4.82 mmol) in t-BuOH (10 mL) was added t-BuONa (284 mg; 2.90 mmol), t-BuBrettPhos (119 mg; 0.24 mmol) and Pd₂dba₃ (233 mg; 0.24 mmol). The suspension was stirred in sealed tube at 90° C. for 1 h. The suspension was concentrated and the residue was purified by column chromatography (silica gel; PE:EA; 1:0 gradient to 1:4; v/v) to afford tert-butyl N-[3-cyano-5-(methylamino)-2-pyridyl]-N-[4-(trifluoromethyl)phenyl]carbamate (670 mg) as a yellow solid.

MS m/z (+ESI): 337.2 [M-(t-Bu)+H]⁺.

¹H-NMR (400 MHz, DMSO-d₆) δ ppm: 8.03 (d, J=3.2 Hz, 1H), 7.70 (d, J=8.8 Hz, 2H), 7.41-7.38 (m, 3H), 6.68 (q, J=5.2 Hz, 1H), 2.74 (d, J=5.2 Hz, 3H), 1.40 (s, 9H).

### Step 3: Preparation of tert-butyl N-[3-(N-hydroxycarbamimidoyl)-5-(methylamino)-2-pyridyl]-N-[4-(trifluoromethyl)phenyl]carbamate

The title compound was prepared as a yellow solid following scheme 1 and in analogy to Example 1 (step 2) using tert-butyl N-[3-cyano-5-(methylamino)-2-pyridyl]-N-[4-(trifluoromethyl)phenyl]carbamate as starting material.

MS m/z (+ESI): 426.3 [M+H]⁺.

### Step 4: Preparation of tert-butyl N-[5-(methylamino)-3-(5-oxo-4H-1,2,4-oxadiazol-3-yl)-2-pyridyl]-N-[4-(trifluoromethyl)phenyl]carbamate

The title compound was prepared as a yellow solid following scheme 1 and in analogy to Example 1 (step 3) using tert-butyl N-[3-(N-hydroxycarbamimidoyl)-5-(methylamino)-2-pyridyl]-N-[4-(trifluoromethyl)phenyl]carbamate as starting material.

MS m/z (+ESI): 452.2 [M+H]⁺.

### Step 5: Preparation of 3-[5-(methylamino)-2-[4-(trifluoromethyl)anilino]-3-pyridyl]-4H-1,2,4-oxadiazol-5-one

The title compound was prepared as a green solid following scheme 1 and in analogy to Example 5 (step 6) using tert-butyl N-[5-(methylamino)-3-(5-oxo-4H-1,2,4-oxadiazol-3-yl)-2-pyridyl]-N-[4-(trifluoromethyl)phenyl]carbamate as starting material and after purification by preparative HPLC.

### Preparation of Example 48: 3-[2-(4-chloroanilino)-4-(2-hydroxyethoxy)-3-pyridyl]-4H-1,2,4-oxadiazol-5-one

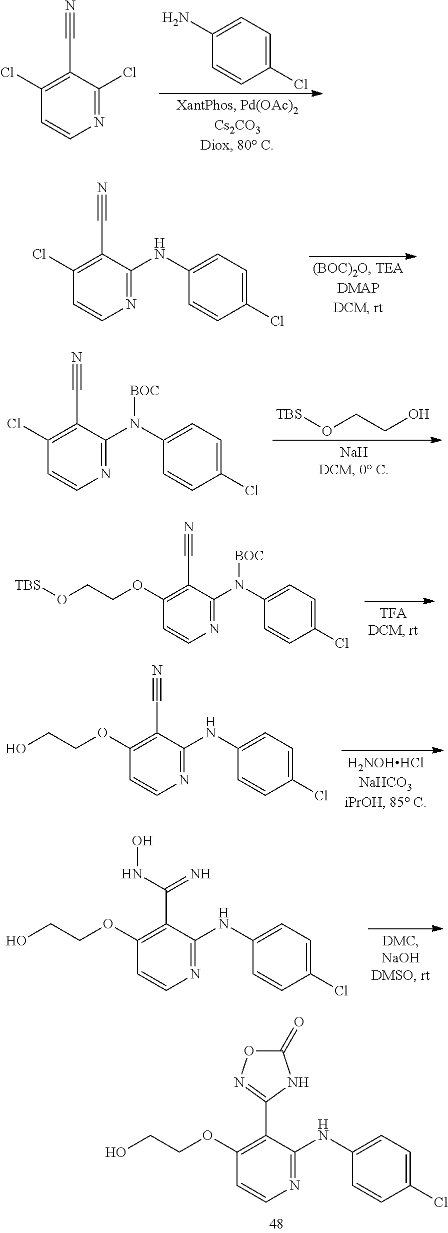

### Step 1: Preparation of 4-chloro-2-(4-chloroanilino)pyridine-3-carbonitrile

The title compound was prepared as a white solid following scheme 1 and in analogy to Example 1 (step 1) using 2,4-dichloronicotinonitrile and 4-chloroaniline as starting materials and after purification by column chromatography (silica gel; PE:EA; 9:1; v/v).

MS m/z (+ESI): 264.1, 266.1 [M+H]⁺.

¹H-NMR (400 MHz, CDCl₃) δ ppm: 8.26 (d, J=5.2 Hz, 1H), 7.53 (d, J=8.8 Hz, 2H), 7.34 (d, J=8.8 Hz, 2H), 7.07 (s, 1H), 6.89 (d, J=5.2 Hz, 1H).

### Step 2: Preparation of tert-butyl N-(4-chloro-3-cyano-2-pyridyl)-N-(4-chlorophenyl)carbamate

The title compound was prepared as a yellow oil following scheme 1 and in analogy to Example 36 (step 1) using 4-chloro-2-(4-chloroanilino)pyridine-3-carbonitrile as starting material.

### Step 3: Preparation of tert-butyl N-[4-[2-[tert-butyl(dimethyl)silyl]oxyethoxy]-3-cyano-2-pyridyl]-N-(4-chlorophenyl)carbamate

A suspension of 2-(t-butyldimethylsiloxy)ethanol (309 mg; 1.67 mmol), NaH (73 mg; 3.03 mmol) in DCM (50 mL) was stirred at 0° C. for 0.5 h. tert-butyl N-(4-chloro-3-cyano-2-pyridyl)-N-(4-chlorophenyl)carbamate (550 mg; 1.51 mmol) was added to the suspension and stirred at 0° C. for 1 h. NH₄Cl solution, saturated in water and EA were added and the organic layer was separated, washed with brine, dried over MgSO₄, filtered and concentrated. The residue was purified by column chromatography (silica gel; PE:EA; 4:1; v/v) to afford tert-butyl N-[4-[2-[tert-butyl(dimethyl)silyl]oxyethoxy]-3-cyano-2-pyridyl]-N-(4-chlorophenyl)carbamate (290 mg) as a yellow oil.

¹H-NMR (400 MHz, CDCl₃) δ ppm: 8.45 (d, J=6.0 Hz, 1H), 7.35-7.29 (m, 4H), 6.92 (d, J=6.0 Hz, 1H), 4.30 (t, J=4.8 Hz, 2H), 4.06 (t, J=4.8 Hz, 2H), 1.50 (s, 9H), 0.90 (s, 9H), 0.12 (s, 6H).

### Step 4: Preparation of 2-(4-chloroanilino)-4-(2-hydroxyethoxy)pyridine-3-carbonitrile

The title compound was prepared as a yellow solid following scheme 1 and in analogy to Example 5 (step 6) using tert-butyl N-[4-[2-[tert-butyl(dimethyl)silyl]oxyethoxy]-3-cyano-2-pyridyl]-N-(4-chlorophenyl)carbamate as starting material

MS m/z (+ESI): 288.2, 290.2 [M+H]⁺.

### Step 5: Preparation of 2-(4-chloroanilino)-N-hydroxy-4-(2-hydroxyethoxy)pyridine-3-carboxamidine

The title compound was prepared as a yellow solid following scheme 1 and in analogy to Example 1 (step 2) using 2-(4-chloroanilino)-4-(2-hydroxyethoxy)pyridine-3-carbonitrile as starting material.

MS m/z (+ESI): 323.1, 325.1 [M+H]⁺.

### Step 6: Preparation of 3-[2-(4-chloroanilino)-4-(2-hydroxyethoxy)-3-pyridyl]-4H-1,2,4-oxadiazol-5-one

The title compound was prepared as a white solid following scheme 1 and in analogy to Example 26 (step 3) using 2-(4-chloroanilino)-N-hydroxy-4-(2-hydroxyethoxy)pyridine-3-carboxamidine as starting material and after purification by preparative HPLC.

### Preparation of Example 52: 3-[4-isopropoxy-2-[4-(trifluoromethyl)anilino]-3-pyridyl]-4H-1,2,4-oxadiazol-5-one

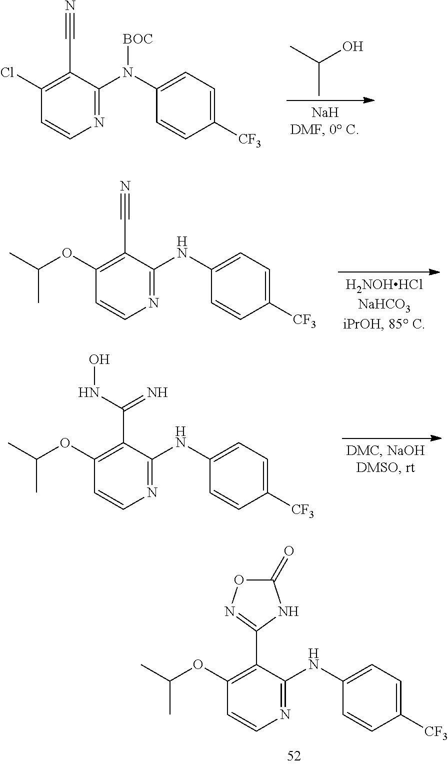

### Step 1: Preparation of 4-isopropoxy-2-[4-(trifluoromethyl)anilino]pyridine-3-carbonitrile

To a solution of i-PrOH (0.1 mL; 1.28 mmol) in DMF (5 mL) was added NaH (93 mg; 2.14 mmol) at 0° C. and the suspension was stirred for 0.5 h. Tert-butyl N-(4-chloro-3-cyano-2-pyridyl)-N-[4-(trifluoromethyl)phenyl]carbamate (500 mg; 1.07 mmol)(intermediate Example 51 step 2) was added. The suspension was stirred for 3 h. NH₄Cl solution, saturated in water and EA were added. The organic layer was separated, washed with brine, dried over MgSO₄ and concentrated to afford 4-isopropoxy-2-[4-(trifluoromethyl)anilino]pyridine-3-carbonitrile (320 mg) as a yellow solid, which was used directly in next step without further purification.

MS m/z (+ESI): 322.1 [M+H]⁺.

¹H-NMR (400 MHz, DMSO-d₆) δ ppm: 9.34 (s, 1H), 8.25 (d, J=6.0 Hz, 1H), 7.76 (d, J=8.6 Hz, 2H), 7.61 (d, J=8.6 Hz, 2H), 6.85 (d, J=6.0 Hz, 1H), 4.90 (m, 1H), 1.34 (d, J=6.0 Hz, 6H).

### Step 2: Preparation of N-hydroxy-4-isopropoxy-2-[4-(trifluoromethyl)anilino]pyridine-3-carboxamidine

The title compound was prepared as a yellow solid following scheme 1 and in analogy to Example 1 (step 2) using 4-isopropoxy-2-[4-(trifluoromethyl)anilino]pyridine-3-carbonitrile as starting material.

MS m/z (+ESI): 355.1 [M+H]⁺.

### Step 3: Preparation of 3-[4-isopropoxy-2-[4-(trifluoromethyl)anilino]-3-pyridyl]-4H-1,2,4-oxadiazol-5-one

The title compound was prepared as a white solid following scheme 1 and in analogy to Example 26 (step 3) using N-hydroxy-4-isopropoxy-2-[4-(trifluoromethyl)anilino]pyridine-3-carboxamidine as starting material and after purification by preparative HPLC.

### Preparation of Example 53: 3-[4-tert-butoxy-2-[4-(trifluoromethyl)anilino]-3-pyridyl]-4H-1,2,4-oxadiazol-5-one

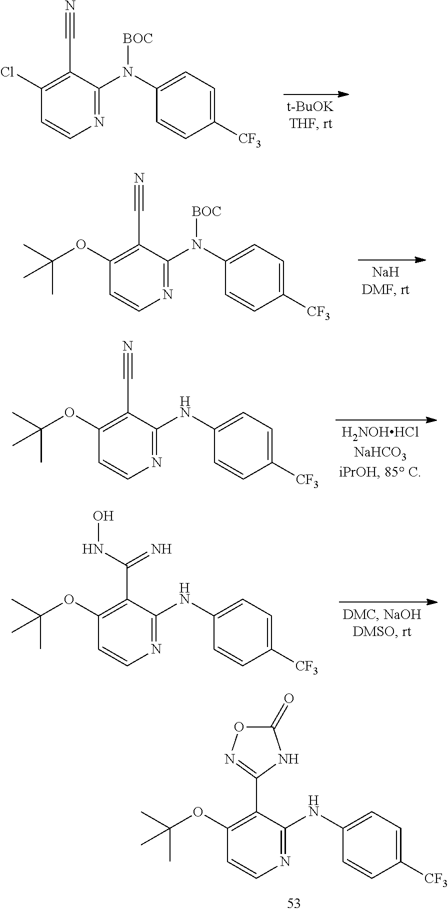

### Step 1: Preparation of tert-butyl N-(4-tert-butoxy-3-cyano-2-pyridyl)-N-[4-(trifluoromethyl)phenyl]carbamate

To a solution of tert-butyl N-(4-chloro-3-cyano-2-pyridyl)-N-[4-(trifluoromethyl)phenyl]carbamate (300 mg; 0.64 mmol)(intermediate Example 51 step 2) in THF (4 mL) was added 4 Å molecular sieves (100 mg) and t-BuOK (120 mg; 1.03 mmol). The suspension was stirred for 5 min. NH₄Cl solution, saturated in water and EA were added. The organic layer was separated, washed with brine, dried over MgSO₄ and concentrated. The residue was purified by column chromatography (silica gel; PE:EA; 1:0 gradient to 1:1; v/v) to afford tert-butyl N-(4-tert-butoxy-3-cyano-2-pyridyl)-N-[4-(trifluoromethyl)phenyl]carbamate (220 mg) as a white solid.

MS m/z (+ESI): 436.1 [M+H]⁺.

### Step 2: Preparation of 4-tert-butoxy-2-[4-(trifluoromethyl)anilino]pyridine-3-carbonitrile

To a solution of tert-butyl N-(4-tert-butoxy-3-cyano-2-pyridyl)-N-[4-(trifluoromethyl)phenyl]carbamate (100 mg; 0.21 mmol) in DMF (2 mL) was added NaH (90 mg; 2.07 mmol) and the suspension was stirred for 16 h. NH₄Cl solution, saturated in water and EA were added. The organic layer was separated, washed with brine, dried over MgSO₄ and concentrated. The residue was purified by column chromatography (silica gel; PE:EA; 1:0 gradient to 4:1; v/v) to afford 4-tert-butoxy-2-[4-(trifluoromethyl)anilino]pyridine-3-carbonitrile (66 mg) as a white solid.

¹H-NMR (400 MHz, CDCl₃) δ ppm: 8.21 (d, J=6.0 Hz, 1H), 7.74 (d, J=8.8 Hz, 2H), 7.60 (d, J=8.8 Hz, 2H), 7.15 (s, 1H), 6.61 (d, J=6.0 Hz, 1H), 1.60 (s, 9H).

### Step 3: Preparation of 4-tert-butoxy-N-hydroxy-2-[4-(trifluoromethyl)anilino]pyridine-3-carboxamidine

The title compound was prepared as a yellow solid following scheme 1 and in analogy to Example 1 (step 2) using 4-tert-butoxy-2-[4-(trifluoromethyl)anilino]pyridine-3-carbonitrile as starting material.

MS m/z (+ESI): 369.1 [M+H]⁺.

### Step 4: Preparation of 3-[4-tert-butoxy-2-[4-(trifluoromethyl)anilino]-3-pyridyl]-4H-1,2,4-oxadiazol-5-one

The title compound was prepared as a white solid following scheme 1 and in analogy to Example 26 (step 3) using 4-tert-butoxy-N-hydroxy-2-[4-(trifluoromethyl)anilino]pyridine-3-carboxamidine as starting material and after purification by preparative HPLC.

### Preparation of Example 54: 3-[4-[2-(2-hydroxyethylamino)ethoxy]-2-[4-(trifluoromethyl)anilino]-3-pyridyl]-4H-1,2,4-oxadiazol-5-one, trifluoroacetate

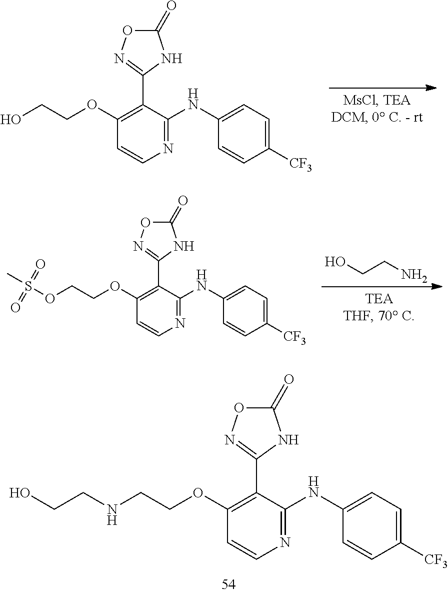

### Step 1: Preparation of 2-[[3-(5-oxo-4H-1,2,4-oxadiazol-3-yl)-2-[4-(trifluoromethyl)anilino]-4-pyridyl]oxy]ethyl methanesulfonate

To a solution of 3-[4-(2-hydroxyethoxy)-2-[4-(trifluoromethyl)anilino]-3-pyridyl]-4H-1,2,4-oxadiazol-5-one (240 mg; 0.57 mmol)(Example 51) and TEA (0.24 mL; 1.70 mmol) in DCM (10 mL) was added MsCl (0.054 mL; 0.68 mmol) at 0° C. The ice-bath was removed and the suspension was stirred for 1 h. The suspension was concentrated to afford 2-[[3-(5-oxo-4H-1,2,4-oxadiazol-3-yl)-2-[4-(trifluoromethyl)anilino]-4-pyridyl]oxy]ethyl methanesulfonate (290 mg) as an off-white semi-sold, which was directly used in next step without further purification.

MS m/z (+ESI): 369.1 [M+H]⁺.

### Step 2: Preparation of 3-[4-[2-(2-hydroxyethylamino)ethoxy]-2-[4-(trifluoromethyl)anilino]-3-pyridyl]-4H-1,2,4-oxadiazol-5-one, trifluoroacetate

In a sealed tube, to a solution of 2-[[3-(5-oxo-4H-1,2,4-oxadiazol-3-yl)-2-[4-(trifluoromethyl)anilino]-4-pyridyl]oxy]ethyl methanesulfonate (150 mg; 0.28 mmol) in THF (2 mL) were added TEA (0.39 mL; 2.77 mmol) and ethanolamine (0.17 mL; 2.77 mmol). The solution was heated to 70° C. and stirred for 18 h. The solution was concentrated and the residue was purified by preparative HPLC to afford 3-[4-[2-(2-hydroxyethylamino)ethoxy]-2-[4-(trifluoromethyl)anilino]-3-pyridyl]-4H-1,2,4-oxadiazol-5-one, trifluoroacetate (6 mg) as a red semi-solid.

### Preparation of Example 55: 3-[4-(2-aminoethoxy)-2-[4-(trifluoromethyl)anilino]-3-pyridyl]-4H-1,2,4-oxadiazol-5-one, trifluoroacetate

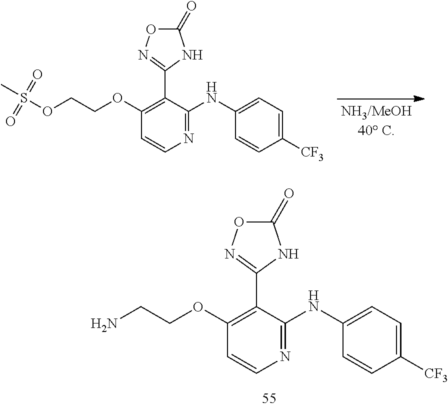 [SMILES: CS(=O)(=O)OCCOc1ccnc(Nc2ccc(C(F)(F)F)cc2)c1-c1noc(=O)[nH]1 ; NCCOc1ccnc(Nc2ccc(C(F)(F)F)cc2)c1-c1noc(=O)[nH]1]

### Step 1: Preparation of 3-[4-(2-aminoethoxy)-2-[4-(trifluoromethyl)anilino]-3-pyridyl]-4H-1,2,4-oxadiazol-5-one, trifluoroacetate

In a sealed tube, a solution of 2-[[3-(5-oxo-4H-1,2,4-oxadiazol-3-yl)-2-[4-(trifluoromethyl)anilino]-4-pyridyl]oxy]ethyl methanesulfonate (430 mg; 0.79 mmol)(intermediate Example 54 step 1) in NH₃ solution, 7M in MeOH (10 mL; 70 mmol) was heated to 40° C. and stirred for 18 h. The solution was concentrated and the residue was purified by preparative HPLC to afford 3-[4-(2-aminoethoxy)-2-[4-(trifluoromethyl)anilino]-3-pyridyl]-4H-1,2,4-oxadiazol-5-one, trifluoroacetate (100 mg) as a white solid.

### Preparation of Example 56: N-[2-[[3-(5-oxo-4H-1,2,4-oxadiazol-3-yl)-2-[4-(trifluoromethyl)anilino]-4-pyridyl]oxy]ethyl]acetamide

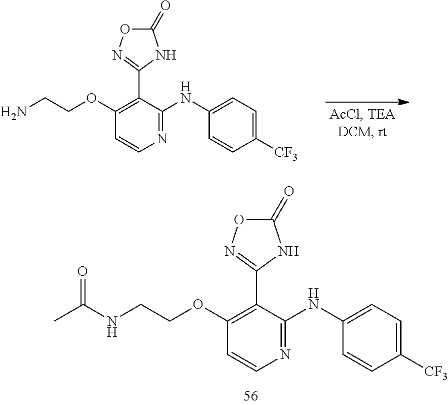

### Step 1: Preparation of N-[2-[[3-(5-oxo-4H-1,2,4-oxadiazol-3-yl)-2-[4-(trifluoromethyl)anilino]-4-pyridyl]oxy]ethyl]acetamide

To a solution of 3-[4-(2-aminoethoxy)-2-[4-(trifluoromethyl)anilino]-3-pyridyl]-4H-1,2,4-oxadiazol-5-one (100 mg; 0.25 mmol)(Example 55) and TEA (0.14 mL; 1.0 mmol) in DCM (3 mL) was added AcCl (0.018 mL; 0.25 mmol) and the suspension was stirred for 0.5 h. The suspension was concentrated and the residue was purified by preparative HPLC to afford N-[2-[[3-(5-oxo-4H-1,2,4-oxadiazol-3-yl)-2-[4-(trifluoromethyl)anilino]-4-pyridyl]oxy]ethyl]acetamide (47 mg) as a white solid.

### Preparation of Example 58: 2-[[3-(5-oxo-4H-1,2,4-oxadiazol-3-yl)-2-[4-(trifluoromethyl)anilino]-4-pyridyl]oxy]acetic acid

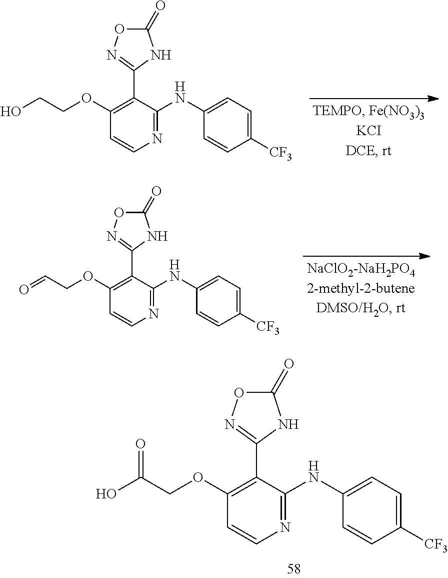 [SMILES: O=c1[nH]c(-c2c(OCCO)ccnc2Nc2ccc(C(F)(F)F)cc2)no1 ; O=CCOc1ccnc(Nc2ccc(C(F)(F)F)cc2)c1-c1noc(=O)[nH]1 ; O=C(O)COc1ccnc(Nc2ccc(C(F)(F)F)cc2)c1-c1noc(=O)[nH]1]

### Step 1: Preparation of 2-[[3-(5-oxo-4H-1,2,4-oxadiazol-3-yl)-2-[4-(trifluoromethyl)anilino]-4-pyridyl]oxy]acetaldehyde

Under normal atmosphere, to a suspension of 3-[4-(2-hydroxyethoxy)-2-[4-(trifluoromethyl)anilino]-3-pyridyl]-4H-1,2,4-oxadiazol-5-one (100 mg; 0.24 mmol)(Example 51) in DCE (5 mL) were added Fe(NO₃)₃ (39 mg; 0.094 mmol), KCl (35 mg; 0.47 mmol) and TEMPO (15 mg; 0.094 mmol). The suspension was stirred for 2 h. DCM and water were added and the organic layer was separated, dried over MgSO₄ and concentrated. The residue was purified by preparative HPLC to afford 2-[[3-(5-oxo-4H-1,2,4-oxadiazol-3-yl)-2-[4-(trifluoromethyl)anilino]-4-pyridyl]oxy]acetaldehyde (70 mg) as an off-white solid.

MS m/z (+ESI): 381.0 [M+H]⁺.

### Step 2: Preparation of 2-[[3-(5-oxo-4H-1,2,4-oxadiazol-3-yl)-2-[4-(trifluoromethyl)anilino]-4-pyridyl]oxy]acetic acid

To a solution of 2-[[3-(5-oxo-4H-1,2,4-oxadiazol-3-yl)-2-[4-(trifluoromethyl)anilino]-4-pyridyl]oxy]-acetaldehyde (50 mg; 0.12 mmol) in DMSO (2 mL) were added 2-methyl-2-butene solution, 2M in THF (1.25 mL; 2.50 mmol) and a solution of NaH₂PO₄ (120 mg; 1.00 mmol) and NaClO₂ (17 mg; 0.19 mmol) in H₂O (0.5 mL). The solution was stirred for 2 h and then purified by preparative HPLC to afford 2-[[3-(5-oxo-4H-1,2,4-oxadiazol-3-yl)-2-[4-(trifluoromethyl)anilino]-4-pyridyl]oxy]acetic acid (40 mg) as a white solid.

### Preparation of Example 59: 2-[[3-(5-oxo-4H-1,2,4-oxadiazol-3-yl)-2-[4-(trifluoromethyl)anilino]-4-pyridyl]oxy]acetamide

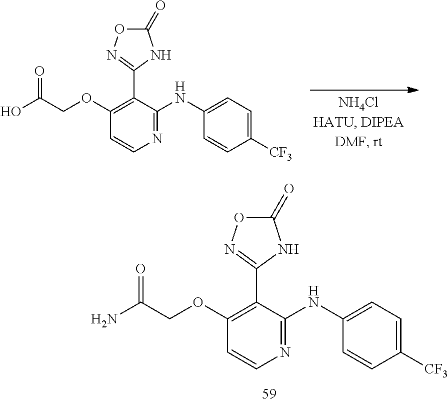

### Step 1: Preparation of 2-[[3-(5-oxo-4H-1,2,4-oxadiazol-3-yl)-2-[4-(trifluoromethyl)anilino]-4-pyridyl]oxy]acetamide

To a solution of 2-[[3-(5-oxo-4H-1,2,4-oxadiazol-3-yl)-2-[4-(trifluoromethyl)anilino]-4-pyridyl]oxy]-acetic acid (20 mg; 0.05 mmol)(Example 58) in DMF (2 mL) were added DIPEA (0.024 mL; 0.14 mmol), NH₄Cl (8 mg; 0.14 mol) and HATU (28 mg; 0.07 mmol). The solution was stirred for 0.5 h and then purified by preparative HPLC to afford 2-[[3-(5-oxo-4H-1,2,4-oxadiazol-3-yl)-2-[4-(trifluoromethyl)anilino]-4-pyridyl]oxy]acetamide (15 mg) as a white solid.

### Preparation of Example 60: 3-[4-(2-morpholinoethoxy)-2-[4-(trifluoromethyl)anilino]-3-pyridyl]-4H-1,2,4-oxadiazol-5-one, trifluoroacetate

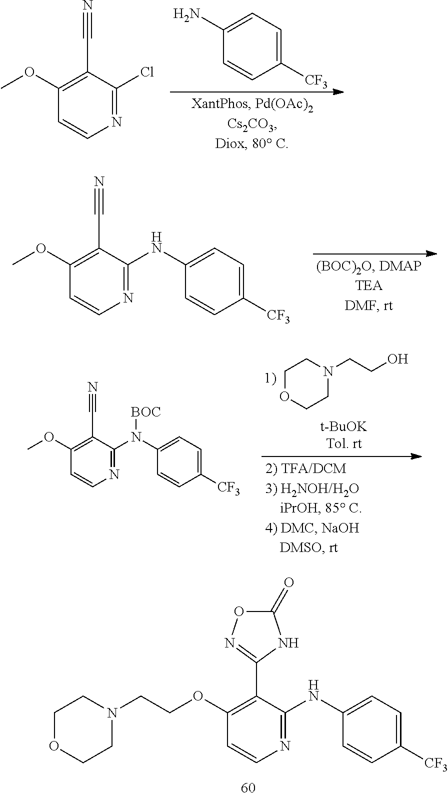

### Step 1: Preparation of 4-methoxy-2-[4-(trifluoromethyl)anilino]pyridine-3-carbonitrile (Intermediate Example 11 step 1)

The title compound was prepared as a white solid following scheme 1 and in analogy to Example 1 (step 1) using 2-chloro-4-methoxy-pyridine-3-carbonitrile and 4-trifluoroaniline as starting materials and after purification by crystallization in EtOH.

MS m/z (+ESI): 294.1 [M+H]⁺.

¹H-NMR (400 MHz, DMSO-d₆) δ ppm: 9.40 (s, 1H), 8.32 (d, J=6.8 Hz, 1H), 7.78 (d, J=8.6 Hz, 2H), 7.64 (d, J=8.6 Hz, 2H), 6.85 (d, J=6.8 Hz, 1H), 4.00 (s, 3H).

### Step 2: Preparation of tert-butyl N-(3-cyano-4-methoxy-2-pyridyl)-N-[4-(trifluoromethyl)phenyl]carbamate

To a solution of 4-methoxy-2-[4-(trifluoromethyl)anilino]pyridine-3-carbonitrile (3 960 mg; 13.2 mmol) in DMF (50 mL) were added (BOC)₂O (5 835 mg; 26.5 mmol), DMAP (165 mg; 1.32 mmol) and TEA (5.58 mL; 39.7 mmol). The solution was stirred for 1 h. EA was added and the mixture was washed with citric acid solution, 10% in water and brine, dried over MgSO4, filtered and concentrated to dryness. The residue was purified by column chromatography (silica gel; c-Hex:EA; 1:0 gradient to 3:1; v/v) to afford tert-butyl N-(3-cyano-4-methoxy-2-pyridyl)-N-[4-(trifluoromethyl)phenyl]carbamate (4 650 mg) as a light yellow solid.

MS m/z (+ESI): 394.2 [M+H]⁺.

### Step 3: Preparation of 3-[4-(2-morpholinoethoxy)-2-[4-(trifluoromethyl)anilino]-3-pyridyl]-4H-1,2,4-oxadiazol-5-one, trifluoroacetate

To a stirred suspension of t-BuOK (180 mg; 1.59 mmol) in Tol. (3 mL) was added N-(2-hydroxyethyl)morpholine (0.28 mL; 2.27 mmol). After stirring for 5 min, a solution of tert-butyl N-(3-cyano-4-methoxy-2-pyridyl)-N-[4-(trifluoromethyl)phenyl]carbamate (300 mg; 0.76 mmol) in Tol. (2 mL) was added. After stirring for 0.5 h, EA was added and the mixture was washed with citric acid solution, 10% in water, brine, dried over MgSO₄, filtered and concentrated. The residue was dissolved in DCM (5 mL) and treated with TFA (0.85 mL; 11.3 mmol). The solution was stirred for 0.5 h and concentrated. The residue was suspended in i-PrOH (3 mL) and hydroxylamine solution, 50% in water (0.66 mL; 11.3 mmol) was added. The suspension was heated to 85° C. and stirred for 3 h. The solution was concentrated and then diluted with EA. The solution was washed with brine, dried over MgSO₄, filtered and concentrated. The new residue was dissolved in DMSO (4 mL) and DMC (0.19 mL; 2.26 mmol) and NaOH (0.09 g; 2.26 mmol) were added. The suspension was stirred for 2 h. EA was added and the solution was washed with water, citric acid solution, 10% in water and brine, dried over MgSO₄, filtered and concentrated. The residue was purified by preparative HPLC to afford 3-[4-(2-morpholinoethoxy)-2-[4-(trifluoromethyl)anilino]-3-pyridyl]-4H-1,2,4-oxadiazol-5-one, trifluoroacetate (40 mg) as a white solid.

### Preparation of Example 67: 3-[4-(4-piperidyloxy)-2-[4-(trifluoromethyl)anilino]-3-pyridyl]-4H-1,2,4-oxadiazol-5-one, trifluoroacetate

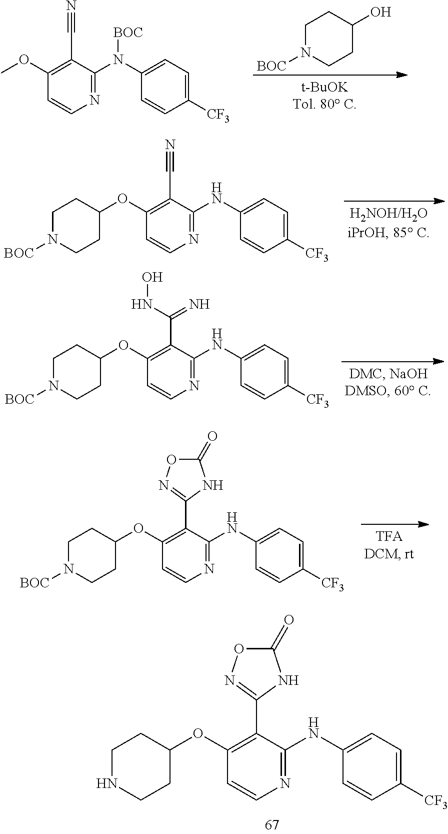

### Step 1: Preparation of tert-butyl 4-[[3-cyano-2-[4-(trifluoromethyl)anilino]-4-pyridyl]oxy]piperidine-1-carboxylate

To a stirred suspension of t-BuOK (180 mg; 1.59 mmol) in Tol. (3 mL) was added N-BOC-4-hydroxypiperidine (470 mg; 2.27 mmol). The suspension was stirred for 10 min and then treated with a solution of tert-butyl N-(3-cyano-4-methoxy-2-pyridyl)-N-[4-(trifluoromethyl)phenyl]carbamate (300 mg; 0.76 mmol)(intermediate Example 60 step 2) in Tol. (2 mL). The mixture was heated to 80° C. and stirred for 1 h. EA was added and the mixture was washed with citric acid solution, 10% in water, brine, dried over MgSO₄, filtered and concentrated. The residue was triturated in EtOH (5 mL) and the suspension was filtered, washed with EtOH and the solid was dried under vacuum to afford tert-butyl 4-[[3-cyano-2-[4-(trifluoromethyl)anilino]-4-pyridyl]oxy]piperidine-1-carboxylate (122 mg) as a white solid.

MS m/z (+ESI): 463.3 [M+H]⁺. 15 ¹H-NMR (400 MHz, DMSO-d₆) δ ppm: 9.41 (s, 1H), 8.29 (d, J=6.0 Hz, 1H), 7.79 (d, J=8.6 Hz, 2H), 7.63 (d, J=8.6 Hz, 2H), 6.95 (d, J=6.0 Hz, 1H), 4.98-4.91 (m, 1H), 3.61-3.55 (m, 2H), 3.38-3.30 (m, 2H, overlap H₂O), 1.98-1.89 (m, 2H), 1.73-1.58 (m, 2H), 1.43 (s, 9H).

### Step 2: Preparation of tert-butyl 4-[[3-(N-hydroxycarbamimidoyl)-2-[4-(trifluoromethyl)anilino]-4-pyridyl]oxy]piperidine-1-carboxylate

To a suspension of tert-butyl 4-[[3-cyano-2-[4-(trifluoromethyl)anilino]-4-pyridyl]oxy]piperidine-1-carboxylate (120 mg; 0.24 mmol) in i-PrOH (2 mL) was added Hydroxylamine solution, 50% in water (0.21 mL; 3.60 mmol). The suspension was heated to 80° C. and stirred for 10 h. Solvent was removed under reduced pressure and the residue was dissolved in EA, washed with citric acid solution, 10% in water, brine, dried over MgSO₄, filtered and concentrated to dryness to afford tert-butyl 4-[[3-(N-hydroxycarbamimidoyl)-2-[4-(trifluoromethyl)anilino]-4-pyridyl]oxy]piperidine-1-carboxylate (130 mg) as a white solid.

MS m/z (+ESI): 496.3 [M+H]⁺.

### Step 3: Preparation of tert-butyl 4-[[3-(5-oxo-4H-1,2,4-oxadiazol-3-yl)-2-[4-(trifluoromethyl)anilino]-4-pyridyl]oxy]piperidine-1-carboxylate

To a solution of tert-butyl 4-[[3-(N-hydroxycarbamimidoyl)-2-[4-(trifluoromethyl)anilino]-4-pyridyl]oxy]piperidine-1-carboxylate (130 mg; 0.24 mmol) in DMSO (2 mL) were added DMC (0.06 mL; 0.71 mmol) and NaOH (29 mg; 0.71 mmol). The suspension was heated to 60° C. and stirred for 4 h. EA was added and the solution was washed with water and brine, dried over MgSO₄, filtered and concentrated. The residue was triturated in Et₂O and the suspension was filtered to afford tert-butyl 4-[[3-(5-oxo-4H-1,2,4-oxadiazol-3-yl)-2-[4-(trifluoromethyl)anilino]-4-pyridyl]oxy]piperidine-1-carboxylate (70 mg) as a white solid.

MS m/z (+ESI): 522.2 [M+H]⁺.

### Step 4: Preparation of 3-[4-(4-piperidyloxy)-2-[4-(trifluoromethyl)anilino]-3-pyridyl]-4H-1,2,4-oxadiazol-5-one, trifluoroacetate

To a suspension of tert-butyl 4-[[3-(5-oxo-4H-1,2,4-oxadiazol-3-yl)-2-[4-(trifluoromethyl)anilino]-4-pyridyl]oxy]piperidine-1-carboxylate (70 mg; 0.11 mmol) in DCM (1 mL) was added dropwise TFA (0.13 mL; 1.71 mmol). The solution was stirred for 5 h and concentrated. The residue was triturated in EA and the suspension was filtered and washed with EA. The solid was dried under vacuum to afford 3-[4-(4-piperidyloxy)-2-[4-(trifluoromethyl)anilino]-3-pyridyl]-4H-1,2,4-oxadiazol-5-one, trifluoroacetate (55 mg) as a white solid.

### BIOLOGICAL EXAMPLES

In Vitro Assay Studies

Thermal Shift Assay

The thermal shift assay (TSA) was utilized to characterize target engagement in vitro based on ligand-dependent thermal stabilization of the protein. N-terminally His-tagged human TEAD2 (amino acids 217-447) expressed and purified from E. coli was purchased from Proteros biostructures (cat no. PR-0365). The melting reactions were performed in white, 96 well qPCR plates (Roche Diagnostics, cat. no. 04 729 692 001) in 20 mM HEPES pH 7, 100 mM NaCl in the presence of 4×SYPRO orange (Sigma, cat. no. S5692). Each well contained 3 μM recombinant TEAD2 and either DMSO (control) or experimental compounds at a final concentration of 7 μM. The total volume was 20 μL and the final DMSO concentration was 1%. The plate was sealed and analyzed in a LightCycler 480 II (Roche Diagnostics) by continuously reading the fluorescence using the 465-580 nm filter set while heating from 25° C. to 95° C. using a linear gradient of 1° C./min. Melting temperatures were determined by numerical differentiation using the LightCycler Thermal Shift Analysis software (Roche Diagnostics). The shifts in melting temperature caused by experimental compounds compared to the control are expressed as ΔTm (Table 2).

TEAD Reporter Gene Assay

A TEAD reporter cell line was purchased from BPS Bioscience (cat. no. 60618). It contains the firefly luciferase gene under control of TEAD responsive elements stably integrated into the human breast cancer MCF7 cells. In proliferating cells, a basal level of unphosphorylated YAP/TAZ resides in the nucleus and drives the TEAD-dependent expression of the luciferase reporter. The cells were cultivated as recommended by the supplier. Inhibition of TEAD reporter gene activity by experimental compounds was measured using white, clear-bottom, 96 well cell culture plates (Greiner Bio-One, cat. no. 655098). The cells were seeded at a density of 20,000 cells per well in 100 μL growth medium and the plates were incubated overnight at 37° C. with 5% CO₂ prior to treatment. Experimental compounds were serially diluted in DMSO to 200× the desired final concentrations. 0.5 μL aliquots of DMSO or the test samples were then mixed into the wells and the cells were further incubated for 24 hours. Luminescence was then measured on a Synergy 4 reader (BioTek) using the ONE-Glo Luciferase Assay System (Promega, cat. no. E6120) according to the manufacturer's instructions. Relative inhibition values were calculated by normalizing the raw data using DMSO-treated cells (0% inhibition) and wells devoid of cells (100% inhibition). IC50 values were calculated by fitting concentration-response data to a sigmoidal 4-parameter logistic model.

Compounds of formula (I) inhibit TEAD reporter gene activity and bind to TEAD as reported in table 2:

TABLE 2

| | IC50 TEAD | |
|---|---|---|
| Example | RGA (nM) | ΔTm (° C.) |
| 1 | 39 | 9.9 |
| 2 | 41 | 10.6 |
| 3 | 90 | 11.5 |
| 4 | 85 | 2 |
| 5 | 117 | 4.5 |
| 6 | 499 | 5 |
| 7 | 2 840 | 0.5 |
| 8 | 1 800 | 7.5 |
| 9 | 52 | 9.2 |
| 10 | 374 | 10.1 |
| 11 | 39 | 6.7 |
| 12 | 44 | 7.5 |
| 13 | 95 | 10.3 |
| 14 | 2 770 | 9.0 |
| 15 | 72 | 11.8 |
| 16 | 552 | 11.1 |
| 17 | 708 | 8.7 |
| 18 | 828 | 2.5 |
| 19 | 131 | 10.1 |
| 20 | 236 | 10.6 |
| 21 | 102 | 2.1 |
| 22 | 70 | 6.1 |
| 23 | 64 | 1.6 |
| 24 | 340 | 3.4 |
| 25 | 117 | 9.6 |
| 26 | 433 | 9.2 |
| 27 | 1 540 | 0.4 |
| 28 | 557 | 6.1 |
| 29 | 503 | 7.3 |
| 30 | 4 910 | 5.3 |
| 31 | 35 | 10.5 |
| 32 | 28 | 10.2 |
| 33 | 127 | 3.6 |
| 34 | 2 120 | 0.4 |
| 35 | 3 830 | 2.5 |
| 36 | 149 | 3.5 |
| 37 | 188 | 12.1 |
| 38 | 39 | 12.6 |
| 39 | 2 820 | 9.3 |
| 40 | 109 | 13.4 |
| 41 | 6 570 | 12.5 |
| 42 | 711 | 5.3 |
| 43 | 930 | 6.6 |
| 44 | 225 | 11.6 |
| 45 | 1 350 | 6.0 |
| 46 | 407 | 7.1 |
| 47 | 72 | 18.0 |
| 48 | 507 | 1.9 |
| 49 | 649 | 0.6 |
| 50 | 37 | 12.2 |
| 51 | 135 | 5.1 |
| 52 | 179 | 5.1 |
| 53 | 815 | 3.7 |
| 54 | 357 | 0.9 |
| 55 | 281 | 1.0 |
| 56 | 767 | 4.3 |
| 57 | 388 | 4.1 |
| 58 | 13 700 | 3.0 |
| 59 | 2 750 | 6.7 |
| 60 | 465 | 1.3 |
| 61 | 147 | 2.9 |
| 62 | 61 | 5.5 |
| 63 | 1 550 | 3.4 |
| 64 | 268 | 4.2 |
| 65 | 81 | 6.0 |
| 66 | 1 470 | 0.4 |
| 67 | 10 700 | 7.1 |

NCI-H226 Tumor Xenograft Efficacy Study

Methodology

Female BALB/c nude mice (GemPharmatech Co., Ltd) were subcutaneously inoculated with 10×106 NCI-H226 tumor cells (ATCC, CRL-5826) in 0.2 mL of PBS mixed with matrigel (1:1) (Corning). Randomization into six groups (n=8 animals per group) was performed when the mean tumor size reached approximately 150 (100-200) mm³, after which drug treatments were started. Animals were dosed daily orally at the specified concentrations using an oral-gavage needle. The dosing volume was 10 mL/kg per mouse, with a volume adjusted to the mouse body weight. The vehicle control (water containing 0.5% hydroxypropyl methyl cellulose (Sigma-Aldrich, #H3785) and 0.5% Tween 80 (Sigma-Aldrich, #P1754) was administered in the same way. Tumor volumes were measured twice weekly in two dimensions using a caliper, and the volume was expressed in mm³ using the formula “V=(L×W\hat{ }2)/2”, where V is the tumor volume, L is the tumor length (the longest tumor dimension) and W is the tumor width (the longest tumor dimension perpendicular to L).

Animal protocols were reviewed and approved by the Institutional Animal Care and Use Committee (IACUC) at CrownBio. During the study, the care and use of animals was conducted in accordance with the regulations of the Association for Assessment and Accreditation of Laboratory Animal Care (AAALAC).

Results

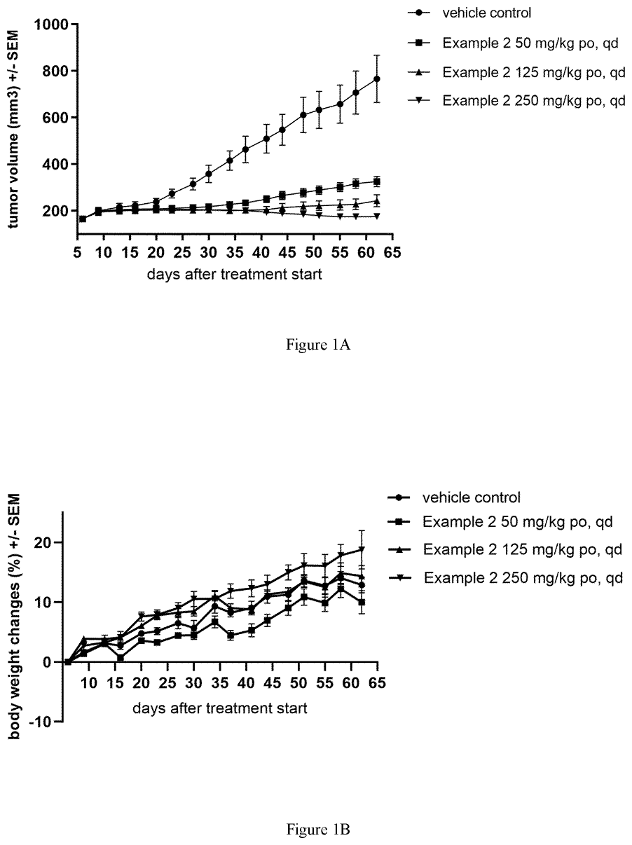

At concentrations of 50, 125 and 250 mg/kg po qd, the compound of Example 2 elicited an anti-tumor response in NCI-H226 xenografts with a final ΔT/C of 0.27, 0.13 and 0.017, respectively, on day 62 (shown in FIG. 1). All comparisons between the vehicle group and treated groups were significantly different (**** p<0.0001). Statistical analyses of the results were performed using the One-Way-ANOVA (Tukey test). ΔT/C=difference between the starting and the final mean tumor size of the drug-treated/vehicle control-treated mice.

MSTO-211H Tumor Xenograft Pharmacodynamic (PD) Studies

Methodology

Female CB. 17 SCID mice (Charles River) were subcutaneously inoculated with 5×106 MSTO-211H tumor cells (ATCC, CRL-2081) in 0.1 mL of PBS mixed with matrigel (1:1) (BD Biosciences). Randomization into two groups was performed when the mean tumor size reached approximately 300-400 mm³, after which drug treatments were started.

Animals were dosed daily orally at the specified concentrations of the compound of Example 2 or the compound of Example 11 using an oral-gavage needle. The dosing volume was 10 mL/kg per mouse, with a volume adjusted to the mouse body weight. The vehicle control (water containing 0.5% hydroxypropyl methyl cellulose (Sigma-Aldrich, #H9262) and 0.5% Tween 80 (Sigma-Aldrich, #P1754)) was administered in the same way.

Animal protocols were reviewed and approved by the IACUC at Charles River Discovery Services North Carolina (CR Discovery Services). The animal care and use program at CR Discovery Services is accredited by the Association for Assessment and Accreditation of Laboratory Animal Care International (AAALAC), which assures compliance with accepted standards for the care and use of laboratory animals.

PD marker analysis was performed on tumors that were collected from animals 3 hours after the last dose was given. The tumors were halved with one half being used for protein analysis and one half being used for RNA analysis. The tumor tissue halves for protein extraction were snap frozen, stored at −80° C. and lysed in cell lysis buffer (Cell Signaling, #9803S) and disrupted with the GentleMACS™ M tubes (MiltenyiBiotec, #130 096 335), exposed to a GentleMACS™ machine (MiltenyiBiotec). The tumor protein lysates were prepared and analyzed using standard Western blot procedures. Antibodies used were CTGF (D8Z8U) (Cell Signaling, #86641) and GAPDH (14C10) (Cell Signaling, #2118), followed by a secondary anti-rabbit HRP (Cell Signaling, #7074). Imaging was performed on a Fusion Solo™ S Imager (Vilber). The tumor halves destined for RNA extraction were pretreated with RNAlater® stabilization solution (ThermoFisher Scientific) according to manufacturer's instructions, snap frozen and stored at −80° C. Pretreated tumor tissues were disrupted as described above. RNA was extracted using the RNeasy® Plus kit (Qiagen, #74136) including the QIAshredder™ step (Qiagen, #79656) according to manufacturer's instructions. For quantitative PCR (qPCR) analyses, RNA was converted into cDNA following manufacturer's instructions (SuperScript™ III First-Strand Synthesis for RT-PCR, Invitrogen, #18080-051). Diluted cDNA was used to assay the expression of each gene with LightCycler® 480 SYBR Green I (Roche, #04 887 352 001). To verify specificity, each PCR was followed by a melting curve analysis. The increase in fluorescence was analyzed with the instrument-specific software (Roche, LightCycler® 480 II), and a mean quantity was calculated from duplicate or triplicate PCRs for each sample using the ΔΔCt method.

Results

The transcriptional TEAD family target CTGF was downregulated on the mRNA and protein level in all compound-treated tumors that were isolated 3 hours after the last dose was given (shown in FIGS. 2 and 3). This result is consistent with direct inhibition of the TEAD family by the described compounds. Specifically, when CTGF mRNA levels were normalized to actin mRNA levels in the respective animal tumors, treatment with the compound of Example 2 at 250 mg/kg po qd×8 resulted in a decrease of CTGF mRNA to 28%. Similar results were observed when CTGF mRNA levels were assessed in animals treated with the compound of Example 11. Here, treatment at 250 mg/kg po qd×5 resulted in CTGF mRNA decrease to 32%.

The invention may be defined by the following numbered paragraphs:

Paragraph 1. A compound of formula (I-c)

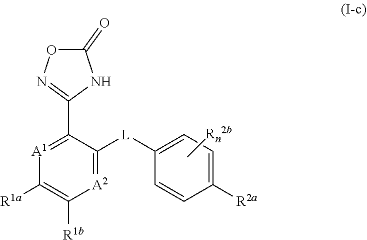

or pharmaceutically acceptable salt thereof, wherein

A1 is —N═ or —C(R3)-;

A2 is —N═;

L is —NH—;

R1a is hydrogen, C1-C3alkyl (n-alkyl) optionally substituted with one R4, or R1a is —NH₂, —NHR5, —N(R5)₂ or —OC1-C3alkyl (n-alkyl);

R1b is hydrogen;

R2a is halogen, C1-C4alkyl, C1-C4haloalkyl, —OC1-C4alkyl or —OC1-C4haloalkyl;

R2b is halogen;

R3 is hydrogen;

R4 is —OH or —NH₂;

each R5 is independently C1-C3alkyl (n-alkyl); and

n is 0 or 1.

Paragraph 2. The compound according to paragraph 1 or pharmaceutically acceptable salt thereof, wherein A1 is —N═.

Paragraph 3. The compound according to paragraph 1 or pharmaceutically acceptable salt thereof, wherein A1 is —C(R3)-.

Paragraph 4. The compound according to paragraph 1 or pharmaceutically acceptable salt thereof, wherein

A1 is —N═ or —C(R3)-;

A2 is —N═;

L is —NH—;

R1a is hydrogen, —CH₃, —CH₂CH₃, —NH₂, —NHCH₃ or —OCH₃;

R1b is hydrogen;

R2a is —CF₃, Cl, Br, —OCH₃, —OCF₃;

R3 is hydrogen;

n is 0.

Paragraph 5. The compound according to paragraph 1 or pharmaceutically acceptable salt thereof, wherein the compound is selected from one of the following compounds:
3-[2-[4-(trifluoromethyl)anilino]-3-pyridyl]-4H-1,2,4-oxadiazol-5-one;
3-[3-[4-(trifluoromethyl)anilino]pyrazin-2-yl]-4H-1,2,4-oxadiazol-5-one;
3-[3-[4-(trifluoromethoxy)anilino]pyrazin-2-yl]-4H-1,2,4-oxadiazol-5-one;
3-[6-amino-3-[4-(trifluoromethyl)anilino]pyrazin-2-yl]-4H-1,2,4-oxadiazol-5-one;
3-[6-methoxy-3-[4-(trifluoromethyl)anilino]pyrazin-2-yl]-4H-1,2,4-oxadiazol-5-one;
3-[6-(hydroxymethyl)-3-[4-(trifluoromethyl)anilino]pyrazin-2-yl]-4H-1,2,4-oxadiazol-5-one;
3-[6-(2-aminoethyl)-3-[4-(trifluoromethyl)anilino]pyrazin-2-yl]-4H-1,2,4-oxadiazol-5-one;
3-[3-[2-chloro-4-(trifluoromethoxy)anilino]pyrazin-2-yl]-4H-1,2,4-oxadiazol-5-one.

Paragraph 6. A compound of formula (I-c) as defined in any one of paragraphs 1 to 5 or pharmaceutically acceptable salt thereof for use in the treatment of a neoplastic disease in a subject selected from a mammal, wherein the compound of formula (I-c) is as defined in any one of paragraphs 1 to 5.

Paragraph 7. A compound of formula (I-c) or pharmaceutically acceptable salt thereof for use according to paragraph 6, wherein the neoplastic disease is cancer.

Paragraph 8. A pharmaceutical composition comprising a compound of formula (I-c) as defined in any one of paragraphs 1 to 5 or pharmaceutically acceptable salt thereof and a pharmaceutically acceptable excipient.

1. A compound of formula (I) 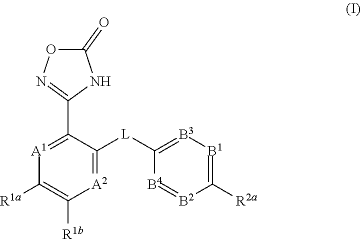
or a pharmaceutically acceptable salt thereof, wherein
A1 is —N═ or —C(R3)=;
A2 is —N═ or —CH═;
L is —NH—;
B1 and B2 are independently —N═ or —C(R2b)=;
B3 and B4 are independently —C(R2b)=;
no more than one R2b on B1, B2, B3 and B4 is other than hydrogen;
R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);
R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;
R1a and R1b may together form a —CH═CH—CH═CH— moiety in which one or two non-adjacent CH are optionally replaced by N;
R2a is halogen, C1-C6alkyl, C1-C6haloalkyl, cyclopropyl, cyclobutyl, —OR6, —NHC(═O)—C3-C6cycloalkyl, Cycle Q, —SF₅ or group Y 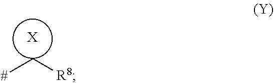
wherein X is a 3- or 4-membered carbocyclic ring and R8 is halogen, cyano, C1-C6alkyl, C1-C6haloalkyl or C1-C6alkyl-CN;
R2b is hydrogen, halogen, methyl, —NH₂, halomethyl, —OCH₃ or —O-halomethyl;
R3 is hydrogen, halogen, C1-C6alkyl, —OC1-C6alkyl, —O—Cycle P, —O—Cycle Q1, —C1-C6alkyl-R9, —OC1-C6alkyl-R9, wherein in each alkyl group or moiety in the foregoing one non-terminal —CH₂— may be replaced by —NH— or —O— and wherein each alkyl group or moiety in the foregoing may be substituted by one or more halogen, wherein R9 is halogen, cyano, hydroxyl, —OC1-C2alkyl, —NH₂, —NH(C1-C2alkyl), —N(C1-C2alkyl)₂, —C(═O)NH₂, —C(═O)NH(C1-C2alkyl), —C(═O)N(C1-C2alkyl)₂, —C(═O)OH, —C(═O)O—C1-C2alkyl, —C(═O)—C1-C2alkyl, —NH(C═O)—C1-C2alkyl, —NH—S(O)₂—C1-C2alkyl, Cycle P or Cycle Q1;
R1a and R3 may together form a —CH₂—CH₂—CH₂— or a —CH₂—CH₂—CH₂—CH₂— moiety in which one of the —CH₂— units is optionally replaced by —NH—;
R4 is —OH or —NH₂;
R6 is C1-C6alkyl, C1-C6haloalkyl or C1-C4alkylene-C3-C6cycloalkyl;
Cycle P is a 3- to 6-membered carbocyclic ring or a 3- to 6-membered heterocyclic ring, each optionally substituted by one to three R10;
Cycle Q is phenyl or 5-6 membered heteroaryl, each optionally substituted by one to three R7;
Cycle Q1 is phenyl or 5-6 membered heteroaryl, each optionally substituted by one to three R7a; and
each R7, R7a and R10 is independently C1-C4alkyl.

2. The compound according to claim 1 or a pharmaceutically acceptable salt thereof, wherein at least one of A1 and A2 is —N═.

3. The compound according to claim 1 or a pharmaceutically acceptable salt thereof, wherein A1 is —C(R3)= and A2 is —N═.

4. The compound according to claim 1 or a pharmaceutically acceptable salt thereof, wherein A1 is —N═ and A2 is —N═.

5. The compound according to claim 1 or a pharmaceutically acceptable salt thereof, wherein A1 is —N═ and A2 is —CH═.

6. The compound according to claim 1 or a pharmaceutically acceptable salt thereof, wherein A1 is —C(R3)=, A2 is —N═ and R3 is other than hydrogen.

7. The compound according to claim 1 or a pharmaceutically acceptable salt thereof, wherein A1 is —C(R3)= and A2 is —CH═.

8. The compound according to claim 1 or a pharmaceutically acceptable salt thereof, wherein B1, B2, B3 and B4 are —CH═.

9. The compound according to any claim 1 or a pharmaceutically acceptable salt thereof, wherein B1 and B2 are —N═ and B3 and B4 are —CH═.

10. The compound according to claim 1 or a pharmaceutically acceptable salt thereof, wherein B1 is —CH═, B2 is —N═ and B3 and B4 are —CH═.

11. The compound according to claim 1 or a pharmaceutically acceptable salt thereof, wherein B1, B2, and B4 are —CH═ and B3 is —C(R2b)= and R2b is other than hydrogen.

12. The compound according to claim 1 or a pharmaceutically acceptable salt thereof, wherein B2, B3 and B4 are —CH═ and B1 is —C(R2b)= and R2b is other than hydrogen.

13. The compound according to claim 1 or a pharmaceutically acceptable salt thereof, wherein at least one of R1a and R1b is hydrogen, or R1a and R1b together form a —CH═CH—CH═CH— moiety.

14. The compound according to claim 1 or a pharmaceutically acceptable salt thereof, wherein
R1a is hydrogen, halogen, C1-C3alkyl (n-alkyl), C1-C3alkyl(n-alkyl)-NH₂, C1-C3alkyl(n-alkyl)-OH, —NH₂, —NH(C1-C3alkyl (n-alkyl)), —NH(C(═O)—C1-C2alkyl), —OC1-C3alkyl (n-alkyl) or C1-C3haloalkyl (n-alkyl);
R1b is hydrogen, C1-C3alkyl (n-alkyl), —OC1-C3alkyl (n-alkyl) or —NH₂; and
at least one of R1a and R1b is hydrogen or R1a and R1b together form a —CH═CH—CH═CH— moiety.

15. The compound according to claim 1 or a pharmaceutically acceptable salt thereof, wherein
R2a is C1-C4alkyl, C1-C4haloalkyl, —OC1-C4alkyl, —OC1-C4haloalkyl, SF₅ or group Y 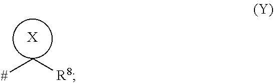
wherein X is 3-membered carbocyclic ring and R8 is halogen, cyano, —C1-C4alkyl, —C1-C4haloalkyl or —C1-C4alkyl-CN; and
R2b is hydrogen or —OCH₃.

16. The compound according to claim 1 or a pharmaceutically acceptable salt thereof, wherein
R3 is hydrogen, halogen, —OC1-C4alkyl, —C1-C4alkyl, —OC1-C4alkyl-R9, —OC1-C2alkyl-NH—C1-C2alkyl-R9 or —O—Cycle P;
R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;
Cycle P is a 5- to 6-membered heterocyclic ring optionally substituted by one to three R10;
Cycle Q1 is 5-6 membered heteroaryl optionally substituted by one to three R7a; and
each R7a and R10 is methyl.

17. The compound according to claim 1 or a pharmaceutically acceptable salt thereof, wherein
A1 is —N═ or —C(R3)=;
A2 is —N═; or —CH═;
L is —NH—;
B1 and B2 are independently —N═ or —C(R2b)=;
B3 and B4 are independently —C(R2b)=;
no more than one R2b on B1, B2, B3 and B4 is other than hydrogen;
R1a is hydrogen, halogen, C1-C3alkyl (n-alkyl), C1-C3alkyl(n-alkyl)-NH₂, C1-C3alkyl(n-alkyl)-OH, —NH₂, —NH(C1-C3alkyl (n-alkyl)), —NH(C(═O)—C1-C2alkyl), —OC1-C3alkyl (n-alkyl) or C1-C3haloalkyl (n-alkyl);
R1b is hydrogen, C1-C3alkyl (n-alkyl), —OC1-C3alkyl (n-alkyl) or —NH₂;
at least one of R1a and R1b is hydrogen or R1a and R1b together form a —CH═CH—CH═CH— moiety;
R2a is C1-C4alkyl, C1-C4haloalkyl, —OC1-C4alkyl, —OC1-C4haloalkyl, —SF₅ or group Y 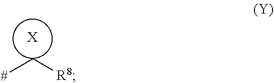
wherein X is 3-membered carbocyclic ring and R8 is halogen, cyano, C1-C4alkyl, C1-C4haloalkyl or —C1-C4alkyl-CN;
R2b is hydrogen or —OCH₃;
R3 is hydrogen, halogen, —OC1-C4alkyl, C1-C4alkyl, —OC1-C4alkyl-R9, —OC1-C2alkyl-NH—C1-C2alkyl-R9 or —O—Cycle P;
R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;
Cycle P is a 5- to 6-membered heterocyclic ring optionally substituted by one to three R10;
Cycle Q1 is 5-6 membered heteroaryl optionally substituted by one to three R7a; and
each R7a and R10 is methyl.

18. The compound according to claim 1 or a pharmaceutically acceptable salt thereof, wherein the compound of formula (I) is a compound of formula (I-a) 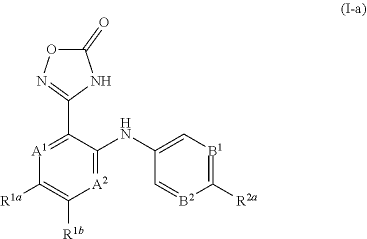
wherein
A1 is —N═ or —C(R3)=;
A2 is —N═ or —CH═;
B1 and B2 are independently —N═ or —C(R2b)= wherein no more than one R2b on B1 and B2 is other than hydrogen;
R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);
R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂;
R1a and R1b may together form a —CH═CH—CH═CH— moiety;
R2a is halogen, C1-C6alkyl, C1-C6haloalkyl, cyclopropyl, cyclobutyl, —OR6, —NHC(═O)—C3-C6cycloalkyl, Cycle Q, —SF₅ or group Y 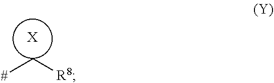
wherein X is a 3- or 4-membered carbocyclic ring and R8 is halogen, cyano, C1-C6alkyl, C1-C6haloalkyl or —C1-C6alkyl-CN;
R2b is hydrogen, halogen, methyl, —NH₂, halomethyl, —OCH₃ or —O-halomethyl;
R3 is hydrogen, halogen, C1-C6alkyl, —OC1-C6alkyl, —O—Cycle P, —O—Cycle Q1, —C1-C6alkyl-R9 or —OC1-C6alkyl-R9, wherein in each alkyl group or moiety in the foregoing one non-terminal —CH₂— may be replaced by —NH— or —O— and wherein each alkyl group or moiety in the foregoing may be substituted by one or more halogen, wherein R9 is halogen, cyano, hydroxyl, —OC1-C2alkyl, —NH₂, —NH(C1-C2alkyl), —N(C1-C2alkyl)₂, —NH—S(O)₂—C1-C2alkyl, Cycle P or Cycle Q1;
R1a and R3 may together form a —CH₂—CH₂—CH₂— or a —CH₂—CH₂—CH₂—CH₂— moiety in which one of the —CH₂— units is optionally replaced by —NH—;
R4 is —OH or —NH₂;
R6 is C1-C6alkyl, C1-C6haloalkyl or C1-C4alkylene-C3-C6cycloalkyl;
Cycle P is a 3- to 6-membered carbocyclic ring or a 3- to 6-membered heterocyclic ring, each optionally substituted by one to three R10;
Cycle Q is phenyl or 5-6 membered heteroaryl, each optionally substituted by one to three R7;
Cycle Q1 is phenyl or 5-6 membered heteroaryl, each optionally substituted by one to three R7a; and
each R7, R7a and R10 is independently C1-C4alkyl.

19. The compound according to claim 18 or a pharmaceutically acceptable salt thereof, wherein in the compound of formula (I-a)
A1 is —N═ or —C(R3)=;
A2 is —N═ or —CH═;
optionally at least one of A1 and A2 is —N═;
B1 and B2 are independently —N═ or —C(R2b)=, wherein no more than one R2b on B1 and B2 is other than hydrogen;
R1a is hydrogen, chloro, methyl or —NH₂;
R1b hydrogen, methyl, methoxy or —NH₂;
at least one of R1a and R1b is hydrogen or R1a and R1b together form a —CH═CH—CH═CH— moiety;
R2a is C1-C4alkyl, C1-C4haloalkyl, —OC1-C4alkyl, —OC1-C4haloalkyl, SF₅ or group Y 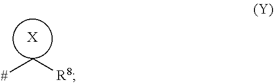
wherein X is 3-membered carbocyclic ring and R8 is halogen, cyano or halomethyl;
R2b is hydrogen or —OCH₃; and
R3 is hydrogen, halogen, —OC1-C4alkyl, —OC1-C3alkyl-halogen or —OC1-C3alkyl-OCH₃.

20. The compound according to claim 1 or a pharmaceutically acceptable salt thereof, wherein R2a is tert-butyl, —CF₃, —O—CF₃, 1-CF₃-cyclopropyl or SF₅.

21. The compound according to claim 1 or a pharmaceutically acceptable salt thereof, wherein the compound of formula (I) is a compound of formula (I-b) 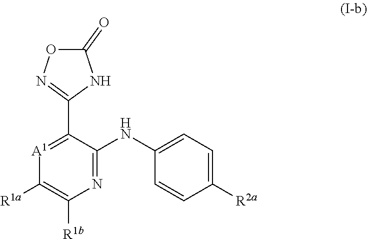
wherein
A1 is —N═ or —C(R3)=;
R1a is hydrogen, chloro, methyl or —NH₂;
R1b hydrogen, methyl, methoxy or —NH₂;
at least one of R1a and R1b is hydrogen;
R2a is tert-butyl, 1-(CF₃)cyclopropyl, —CF₃, —O—CF₃ or SF₅; and
R3 is hydrogen, halogen, —OC1-C4alkyl, —OC1-C3alkyl-halogen or —OC1-C3alkyl-OCH₃.

22. A compound selected from one of the following compounds or a pharmaceutically acceptable salt thereof:
3-[2-[4-(trifluoromethyl)anilino]-3-pyridyl]-4H-1,2,4-oxadiazol-5-one;
3-[3-[4-(trifluoromethyl)anilino]pyrazin-2-yl]-4H-1,2,4-oxadiazol-5-one;
3-[3-[4-(trifluoromethoxy)anilino]pyrazin-2-yl]-4H-1,2,4-oxadiazol-5-one;
3-[6-amino-3-[4-(trifluoromethyl)anilino]pyrazin-2-yl]-4H-1,2,4-oxadiazol-5-one;
3-[6-methoxy-3-[4-(trifluoromethyl)anilino]pyrazin-2-yl]-4H-1,2,4-oxadiazol-5-one;
3-[6-(hydroxymethyl)-3-[4-(trifluoromethyl)anilino]pyrazin-2-yl]-4H-1,2,4-oxadiazol-5-one;
3-[6-(2-aminoethyl)-3-[4-(trifluoromethyl)anilino]pyrazin-2-yl]-4H-1,2,4-oxadiazol-5-one;
3-[3-[2-chloro-4-(trifluoromethoxy)anilino]pyrazin-2-yl]-4H-1,2,4-oxadiazol-5-one;
3-[3-[4-(trifluoromethyl)anilino]-2-pyridyl]-4H-1,2,4-oxadiazol-5-one;
3-[5-(trifluoromethyl)-2-[4-(trifluoromethyl)anilino]-3-pyridyl]-4H-1,2,4-oxadiazol-5-one;
3-[4-methoxy-2-[4-(trifluoromethyl)anilino]-3-pyridyl]-4H-1,2,4-oxadiazol-5-one;
3-[5-methoxy-3-[4-(trifluoromethyl)anilino]pyrazin-2-yl]-4H-1,2,4-oxadiazol-5-one;
3-[4-fluoro-2-[4-(trifluoromethyl)anilino]-3-pyridyl]-4H-1,2,4-oxadiazol-5-one;
3-[3-[4-(cyclopropylmethoxy)anilino]pyrazin-2-yl]-4H-1,2,4-oxadiazol-5-one;
3-[3-(4-tert-butylanilino)pyrazin-2-yl]-4H-1,2,4-oxadiazol-5-one;
3-[3-(4-isopropylanilino)pyrazin-2-yl]-4H-1,2,4-oxadiazol-5-one;
3-[3-(4-ethylanilino)pyrazin-2-yl]-4H-1,2,4-oxadiazol-5-one;
3-[3-(4-thiazol-2-ylanilino)pyrazin-2-yl]-4H-1,2,4-oxadiazol-5-one;
3-[3-[3-fluoro-4-(trifluoromethyl)anilino]pyrazin-2-yl]-4H-1,2,4-oxadiazol-5-one;
3-[3-[3-chloro-4-(trifluoromethyl)anilino]pyrazin-2-yl]-4H-1,2,4-oxadiazol-5-one;
3-[3-[2-methoxy-4-(trifluoromethyl)anilino]pyrazin-2-yl]-4H-1,2,4-oxadiazol-5-one;
3-[3-[3-methoxy-4-(trifluoromethyl)anilino]pyrazin-2-yl]-4H-1,2,4-oxadiazol-5-one;
3-[3-[[2-(trifluoromethyl)pyrimidin-5-yl]amino]pyrazin-2-yl]-4H-1,2,4-oxadiazol-5-one;
3-[3-[[6-(trifluoromethyl)-3-pyridyl]amino]pyrazin-2-yl]-4H-1,2,4-oxadiazol-5-one;
3-[3-[[2-(trifluoromethyl)pyrimidin-5-yl]amino]pyrazin-2-yl]-4H-1,2,4-oxadiazol-5-one;
3-[4,6-dimethyl-2-[4-(trifluoromethyl)anilino]-3-pyridyl]-4H-1,2,4-oxadiazol-5-one;
N-[4-[[3-(5-oxo-4H-1,2,4-oxadiazol-3-yl)pyrazin-2-yl]amino]phenyl]cyclobutanecarboxamide;
3-[4-methyl-2-[4-(trifluoromethyl)anilino]-3-pyridyl]-4H-1,2,4-oxadiazol-5-one;
3-[3-(4-phenylanilino)pyrazin-2-yl]-4H-1,2,4-oxadiazol-5-one;
3-[3-[4-chloro-3-(trifluoromethyl)anilino]pyrazin-2-yl]-4H-1,2,4-oxadiazol-5-one;
3-[5-methyl-3-[4-(trifluoromethyl)anilino]pyrazin-2-yl]-4H-1,2,4-oxadiazol-5-one;
3-[6-methyl-3-[4-(trifluoromethyl)anilino]pyrazin-2-yl]-4H-1,2,4-oxadiazol-5-one;
3-[3-[3-amino-4-(trifluoromethyl)anilino]pyrazin-2-yl]-4H-1,2,4-oxadiazol-5-one;
3-[3-[2-amino-4-(trifluoromethyl)anilino]pyrazin-2-yl]-4H-1,2,4-oxadiazol-5-one;
N-[6-(5-oxo-4H-1,2,4-oxadiazol-3-yl)-5-[4-(trifluoromethyl)anilino]pyrazin-2-yl]acetamide;
3-[5-amino-3-[4-(trifluoromethyl)anilino]pyrazin-2-yl]-4H-1,2,4-oxadiazol-5-one;
3-[3-[4-(trifluoromethyl)anilino]quinoxalin-2-yl]-4H-1,2,4-oxadiazol-5-one;
3-[2-[4-(trifluoromethyl)anilino]phenyl]-4H-1,2,4-oxadiazol-5-one;
3-[3-(4-cyclopropylanilino)pyrazin-2-yl]-4H-1,2,4-oxadiazol-5-one;
3-[3-[4-[1-(trifluoromethyl)cyclopropyl]anilino]pyrazin-2-yl]-4H-1,2,4-oxadiazol-5-one;
3-[3-(4-cyclobutylanilino)pyrazin-2-yl]-4H-1,2,4-oxadiazol-5-one;
1-[4-[[3-(5-oxo-4H-1,2,4-oxadiazol-3-yl)pyrazin-2-yl]amino]phenyl]cyclobutanecarbonitrile;
3-[3-(4-chloroanilino)pyrazin-2-yl]-4H-1,2,4-oxadiazol-5-one;
3-[5-chloro-2-[4-(trifluoromethyl)anilino]-3-pyridyl]-4H-1,2,4-oxadiazol-5-one;
3-[5-amino-2-[4-(trifluoromethyl)anilino]-3-pyridyl]-4H-1,2,4-oxadiazol-5-one;
3-[5-(methylamino)-2-[4-(trifluoromethyl)anilino]-3-pyridyl]-4H-1,2,4-oxadiazol-5-one;
3-[5,6-dimethyl-2-[4-(trifluoromethyl)anilino]-3-pyridyl]-4H-1,2,4-oxadiazol-5-one;
3-[2-(4-chloroanilino)-4-(2-hydroxyethoxy)-3-pyridyl]-4H-1,2,4-oxadiazol-5-one;
3-[3-[4-(trifluoromethyl)anilino]-6,7-dihydro-5H-cyclopenta[c]pyridin-4-yl]-4H-1,2,4-oxadiazol-5-one;
3-[3-[4-(pentafluoro-λ6-sulfanyl)anilino]pyrazin-2-yl]-4H-1,2,4-oxadiazol-5-one;
3-[4-(2-hydroxyethoxy)-2-[4-(trifluoromethyl)anilino]-3-pyridyl]-4H-1,2,4-oxadiazol-5-one;
3-[4-isopropoxy-2-[4-(trifluoromethyl)anilino]-3-pyridyl]-4H-1,2,4-oxadiazol-5-one;
3-[4-tert-butoxy-2-[4-(trifluoromethyl)anilino]-3-pyridyl]-4H-1,2,4-oxadiazol-5-one;
3-[4-[2-(2-hydroxyethylamino)ethoxy]-2-[4-(trifluoromethyl)anilino]-3-pyridyl]-4H-1,2,4-oxadiazol-5-one;
3-[4-(2-aminoethoxy)-2-[4-(trifluoromethyl)anilino]-3-pyridyl]-4H-1,2,4-oxadiazol-5-one;
N-[2-[[3-(5-oxo-4H-1,2,4-oxadiazol-3-yl)-2-[4-(trifluoromethyl)anilino]-4-pyridyl]oxy]ethyl]acetamide;
N-[2-[[3-(5-oxo-4H-1,2,4-oxadiazol-3-yl)-2-[4-(trifluoromethyl)anilino]-4-pyridyl]oxy]ethyl]methanesulfonamide;
2-[[3-(5-oxo-4H-1,2,4-oxadiazol-3-yl)-2-[4-(trifluoromethyl)anilino]-4-pyridyl]oxy]acetic acid;
2-[[3-(5-oxo-4H-1,2,4-oxadiazol-3-yl)-2-[4-(trifluoromethyl)anilino]-4-pyridyl]oxy]acetamide;
3-[4-(2-morpholinoethoxy)-2-[4-(trifluoromethyl)anilino]-3-pyridyl]-4H-1,2,4-oxadiazol-5-one;
3-[4-[2-(2-pyridyl)ethoxy]-2-[4-(trifluoromethyl)anilino]-3-pyridyl]-4H-1,2,4-oxadiazol-5-one;
3-[4-(2-methoxyethoxy)-2-[4-(trifluoromethyl)anilino]-3-pyridyl]-4H-1,2,4-oxadiazol-5-one;
3-[4-[(1-methylcyclopropyl)methoxy]-2-[4-(trifluoromethyl)anilino]-3-pyridyl]-4H-1,2,4-oxadiazol-5-one;
3-[4-tetrahydropyran-4-yloxy-2-[4-(trifluoromethyl)anilino]-3-pyridyl]-4H-1,2,4-oxadiazol-5-one;
3-[4-(2-fluoroethoxy)-2-[4-(trifluoromethyl)anilino]-3-pyridyl]-4H-1,2,4-oxadiazol-5-one;
3-[4-(3-methoxy-3-methyl-butoxy)-2-[4-(trifluoromethyl)anilino]-3-pyridyl]-4H-1,2,4-oxadiazol-5-one; and
3-[4-(4-piperidyloxy)-2-[4-(trifluoromethyl)anilino]-3-pyridyl]-4H-1,2,4-oxadiazol-5-one.

23-24. (canceled)

25. A method of treating a neoplastic disease in a subject selected from a mammal comprising administering a compound of formula (I) as defined in claim 1 or a pharmaceutically acceptable salt thereof in a therapeutically acceptable amount to said subject.

26. The method according to claim 25, wherein the neoplastic disease is cancer.

27. The method according to claim 25, wherein the cancer is mediated by modulation of the interaction of YAP/TAZ with TEAD.

28. The method according to claim 25, wherein the subject is human.

29. A pharmaceutical composition comprising a compound of formula (I) as defined in claim 1 or a pharmaceutically acceptable salt thereof and a pharmaceutically acceptable excipient.

30. A compound of formula (Int-I) 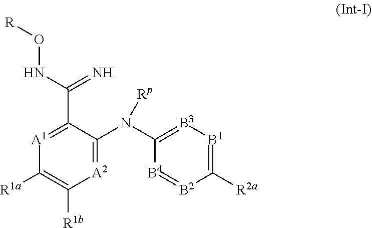
wherein A1, A2, B1, B2, B3, B4, R1a, R1b and R2a are as defined for compounds of formula (I) in any one of claims 1 to 21, and wherein when R1a, R1b and R2a include an amine moiety, the amine moiety may be protected by a protecting group such as tert-butyl carbamate (Boc), 9-Fluorenylmethylcarbamate (Fmoc), benzyl carbamate, acetamide, trifluoroacetamide, phthalimide, benzylamine, triylamine, benzylideneamine or p-Toluenesulfonamide;
wherein R is hydrogen or —C(═O)—O—C1-C4alkyl, wherein the alkyl is optionally substituted with one to three halogen, or R is —C(═O)—O-phenyl, wherein the phenyl is optionally substituted with an NO₂ group; and
R^{p} is hydrogen or a protecting group such as tert-butyl carbamate (Boc), 9-Fluorenylmethylcarbamate (Fmoc), benzyl carbamate, acetamide, trifluoroacetamide, phthalimide, benzylamine, triylamine, benzylideneamine or p-Toluenesulfonamide;
and wherein the compound of formula (Int-I) is not the following compound: Benzenecarboximidamide, N-hydroxy-2-[(4-methylphenyl)amino]-.

31. The compound of formula (Int-I) according to claim 30, wherein
A1 is —N═ and A2 is —N═; or
A1 is —N═ and A2 is —CH═; or
A1 is —C(R3)= and A2 is —N═; or
A1 is —C(R3)= and A2 is —CH═.

32. A compound of formula (Int-II) 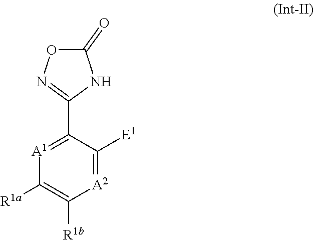
wherein A1, A2, R1a and R1b are as defined for compounds of formula (I) in any one of claims 1 to 21, and wherein E1 is halogen, or a leaving group selected from a perfluoroalkylsulfonate and a sulfonic acid ester and wherein the compound is not the following compounds:
1,2,4-Oxadiazol-5(2H)-one, 3-(3-chloro-2-quinoxalinyl)-;
1,2,4-Oxadiazol-5(2H)-one, 3-(3-chloro-2-pyridinyl)-;
1,2,4-Oxadiazol-5(2H)-one, 3-(2-bromo-6-chlorophenyl)-;
1,2,4-Oxadiazol-5(2H)-one, 3-(2,5-dibromophenyl)-;
1,2,4-Oxadiazol-5(2H)-one, 3-(2-iodophenyl)-;
1,2,4-Oxadiazol-5(2H)-one, 3-(2-chloro-6-fluorophenyl)-;
1,2,4-Oxadiazol-5(2H)-one, 3-(2-chloro-4-methylphenyl)-;
1,2,4-Oxadiazol-5(2H)-one, 3-(2-bromo-4-methoxyphenyl)-;
1,2,4-Oxadiazol-5(2H)-one, 3-(2-bromo-4,5-difluorophenyl)-;
1,2,4-Oxadiazol-5(2H)-one, 3-(5-bromo-2-chlorophenyl)-;
1,2,4-Oxadiazol-5(2H)-one, 3-(2,5-dichlorophenyl)-;
1,2,4-Oxadiazol-5(2H)-one, 3-(2-bromo-5-fluorophenyl)-;
1,2,4-Oxadiazol-5(2H)-one, 3-(2-bromo-4-fluorophenyl)-;
1,2,4-Oxadiazol-5(2H)-one, 3-(2-bromo-5-methoxyphenyl)-;
1,2,4-Oxadiazol-5(2H)-one, 3-(4-bromo-2-chlorophenyl)-;
1,2,4-Oxadiazol-5(2H)-one, 3-(2-chloro-6-methoxyphenyl)-;
1,2,4-Oxadiazol-5(2H)-one, 3-(2-bromo-4-methylphenyl)-;
1,2,4-Oxadiazol-5(2H)-one, 3-(2-bromo-4-chlorophenyl)-;
1,2,4-Oxadiazol-5(2H)-one, 3-(2-bromo-5-chlorophenyl)-;
1,2,4-Oxadiazol-5(2H)-one, 3-(2-chloro-4-fluorophenyl)-;
1,2,4-Oxadiazol-5(2H)-one, 3-(4-amino-2,5-difluorophenyl)-;
1,2,4-Oxadiazol-5(2H)-one, 3-(4-amino-2,6-difluorophenyl)-;
1,2,4-Oxadiazol-5(2H)-one, 3-(4-amino-2-fluorophenyl)-;
1,2,4-Oxadiazol-5(2H)-one, 3-(4-amino-2-chlorophenyl)-;
1,2,4-Oxadiazol-5(2H)-one, 3-(2,4-dichlorophenyl)-;
1,2,4-Oxadiazol-5(2H)-one, 3-(2-bromophenyl)-;
1,2,4-Oxadiazol-5(2H)-one, 3-(2-bromo-4,5-dimethoxyphenyl)-;
1,2,4-Oxadiazol-5(2H)-one, 3-(2,6-difluoro-3-methoxyphenyl)-;
1,2,4-Oxadiazol-5(2H)-one, 3-(2,6-dichlorophenyl)-;
1,2,4-Oxadiazol-5(2H)-one, 3-(2-chlorophenyl)-; and
1,2,4-Oxadiazol-5(2H)-one, 3-(2-fluorophenyl)-.

33. The compound of formula (Int-II) according to claim 32, wherein at least one of A1 and A2 is —N═.

34. The compound of formula (Int-II) according to claim 32, wherein
A1 and A2 are —N═;
R1a is hydrogen, halogen, C1-C3alkyl (n-alkyl) optionally substituted with one R4, or R1a is —NH₂, —NH(C1-C3alkyl (n-alkyl)), —NH(C(═O)—C1-C2alkyl), —N(C1-C3alkyl (n-alkyl))₂, —OC1-C3alkyl (n-alkyl), C1-C3haloalkyl (n-alkyl) or —OC1-C3haloalkyl (n-alkyl);
R1b is hydrogen, halogen, C1-C3alkyl (n-alkyl), —OC1-C3alkyl (n-alkyl), —NH₂, —NH(C1-C3alkyl (n-alkyl)) or —N(C1-C3alkyl (n-alkyl))₂; and
R4 is —OH or —NH₂.
